(19) United States
(12) Patent Application Publication
(10) Pub. No.: US 2003/0190368 A1
(43) Pub. Date: Oct. 9, 2003

(54) METHODS OF DIAGNOSIS AND TRIAGE USING CELL ACTIVATION MEASURES

(76) Inventors: ROLAND STOUGHTON, SAN DIEGO, CA (US); GEERT SCHMID-SCHONBEIN, DEL MAR, CA (US); TONY E. HUGLI, SAN DIEGO, CA (US); ERIK KISTLER, LA JOLLA, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 09/038,894

(22) Filed: Mar. 11, 1998

(51) Int. Cl. A61K 35/39; A61K 35/37

(52) U.S. Cl. 424/556; 424/550; 424/551

(57) ABSTRACT

Diagnostic methods that rely on the use of one or more assays that assess cellular activation are provided. The assays are performed on whole blood or leukocytes, and indicate individually or in combination the level of cardiovascular cell activation, which is pivotal in many chronic and acute disease states. These results of the assays are used within a clinical framework to support therapeutic decisions such as: further testing for infectious agents, anti-oxidant or anti-adhesion therapy, postponement and optimal re-scheduling of high-risk surgeries, classifying susceptibility to and progression rates of chronic disease such as diabetes, atherogenesis, and venous insufficiency; extreme interventions in trauma cases of particularly high risk and activation-lowering therapies. Also provided is as composition derived from a pancreatic homogenate that contains circulating cell activating factors, which can serve as targets for drug screening to identify drug candidates for use in activation lowering therapies. Methods for lowering cell activation by administering protease inhibitors, particularly serine protease inhibitors, are also provided.

Some of the work described herein was supported by U.S.P.H.S. Grant HL-43024. The government, thus, may have some rights in the subject matter disclosed herein.

### FIELD OF THE INVENTION

The present invention relates to the identification of cellular activation factors and the use of cellular activation as a diagnostic marker.

### BACKGROUND OF THE INVENTION

Immune Response and Cellular Activation

Immunity is concerned with the recognition and disposal of foreign (i.e. non-self) antigenic material present in the body. Typically the antigens are particulate matter, such as cells and bacteria, large proteins, polysaccharides and other macromolecules that are recognized by the immune system. Once the antigenic material is recognized as “non-self” by the immune system, natural (non-specific) and/or adaptive immune responses can be initiated and maintained by the action of specific immune cells, antibodies and the complement system.

An immune response can be carried out by the immune system by means of natural or adaptive mechanisms, each of which are composed of cell-mediated and humoral elements. Natural mechanisms, which provide natural immunity, are those that mediate or are involved in substantially non-specific immune reactions, which involve the complement system and myeloid cells alone, such as macrophages, mast cells and polymorphonuclear leukocytes (PMN), reacting to certain bacteria, viruses, tissue damage and other antigens.

Natural mechanisms of immune response involve phagocytosis macrophages and PMN whereby foreign material or antigen is engulfed and disposed of by these cells. In addition, macrophages can kill some foreign cells through its cytotoxic effects. The complement system, which is also involved in natural immunity, is made up of various peptides and enzymes which can attach to foreign material or antigen and thereby promote phagocytosis by macrophages and PMN, or enable cell lysis or inflammatory effects to take place.

Adaptive mechanisms for immune responses are mediated by lymphocytes (T and B cells) and antibodies that selectively respond. These mechanisms lead to a specific memory and a permanently altered pattern of response in adaptation to the environment. Adaptive immunity can be provided by the lymphocytes and antibodies alone or by the interaction of lymphocytes and antibodies with the complement system and myeloid cells of the natural mechanisms of immunity. The antibodies provide the humoral element of the adaptive immune response and the T-cells provide cell-mediated element of the adaptive immune response.

Adaptive mechanisms of immune response involve the actions against specific antigens by antibodies secreted by B-lymphocytes (or B-cells) as well as the actions of various T-lymphocytes (or T-cells) on a specific antigen, on B-cells, on other T-cells and on macrophages.

Lymphocytes are small cells that circulate from the blood, through the tissues, and back to the blood via the lymph system. There are two major subpopulations of lymphocytes called B-cells and T-cells. B-cells and T-cells are derived from the same lymphoid stem cell with the B-cells differentiating in the bone marrow and the T-cells differentiating in the thymus. The lymphocytes possess certain restricted receptors which permit each cell to respond to a specific antigen. This provides the basis for the specificity of the adaptive immune response. In addition, lymphocytes have a relatively long lifespan and have the ability to proliferate clonally upon receiving the proper signal. This property provides the basis for the memory aspect of the adaptive immune response. B-cells are the lymphocytes responsible for the humoral aspect of adaptive immunity. In response to recognition of a specific foreign antigen, a B-cell will secrete a specific antibody which binds to that specific antigen. The antibody neutralizes the antigen, in the case of toxins, or promotes phagocytosis, in the case of other antigens. Antibodies also are involved in the activation of the complement system which further escalates the immune response toward the invading antigen.

Antibodies, which have a wide range of specificities for different antigens are serum globulins are secreted by B-cells in response to the recognition of specific antigens and provide a variety of protective responses. Antibodies can bind to and neutralize bacterial toxins and can bind to the surface of viruses, bacteria, or other cells recognized as “non-self” and promote phagocytosis by PMN and macrophages. In addition, antibodies can activate the complement system which further augments the immune response against the specific antigen.which are responsible for the humoral aspect of adaptive immunity,

T-cells are the lymphocytes responsible for the cell-mediated aspect of adaptive immunity. There are three major types of T-cells, the cytotoxic T-cells, helper T-cells and the suppressor T-cells. The cytotoxic T-cells detects and destroys cells infected with a specific virus antigen. Helper T-cells have a variety of regulatory functions. Helper T-cells, upon identification of a specific antigen, promote or enhance an antibody response to the antigen by the appropriate B-cell and promote or enhance phagocytosis of the antigen by macrophages. Suppressor T-cells have the effect of suppressing an immune response directed toward a particular antigen.

The cell-mediated immune response is controlled and monitored by the T-cells through a variety of regulatory messenger compounds secreted by the myeloid cells and the lymphocyte cells. Through the secretion of these regulatory messenger compounds, the T-cells can regulate the proliferation and activation of other immune cells such as B-cells, macrophages, PMN and other T-cells. For example, upon binding a foreign antigen, a macrophage or other antigen presenting cell can secrete interleukin-1 (IL-1) which activates the helper T-cells. T-cells in turn secrete certain lymphokines, including interleukin-2 (IL-2) and γ-interferon, each of which have a variety of regulatory effects in the cell-mediated immune response.

Lymphokines are a large family of molecules produced by T-cells (and sometimes B-cells) including IL-2, which promotes the clonal proliferation of T-cells; MAF or macrophage activation factor, which increases many macrophage functions including phagocytosis, intracellular killing and secretion of various cytotoxic factors; activating factors that increase many functions of the PMN including phagocytosis; MIF or macrophage migration factor, which by restricting the movement of macrophages, concentrates them in the vicinity of the T-cell; γ-interferon, which is produced by the activated T-cell and is capable of producing a wide range of effects on many cells including inhibition of virus replication, induction of expression of class II histocompatibility molecules allowing these cells to become active in antigen binding and presentation, activation of macrophages, inhibition of cell growth, induction of differentiation of a number of myeloid cell lines.

Activated macrophages and PMNs, which provide an enhanced immune response as part of the cell-mediated adaptive immunity, exhibit increased production of reactive oxygen intermediates. This increased production of reactive oxygen intermediates, or respiratory burst, is known as “priming”. Certain lymphokines, such as γ-interferon, trigger this respiratory burst of reactive oxygen intermediates in macrophages and PMNs. Thus, lymphokines, such as γ-interferon, which are secreted by the T-cells provide an activation of these macrophages and PMNs, resulting in an enhanced cell-mediated immune response.

Neutrophil Activation

Thus, cellular activation is a normal physiological response that is essential for survival. Inappropriate or excessive activation, however, may also be related to certain acute and chronic diseases. The organism itself is often responsible for its own demise, through the inappropriate stimulation of various defense strategies involving inflammatory cells and the immune system. The first inflammatory cells to be upregulated in these conditions are polymorphonucleated (PMN) cells, or neutrophils. These cells, which include 60% of the circulating pool of leukocytes in humans, constitute a formidable line of defense against invading pathogens. When activated, they produce a number of cytotoxic components including oxygen free radicals and proteases designed to destroy and degrade invading bacteria. When unregulated, secreted neutrophil products may also kill cells in the body and destroy tissue.

Although inappropriate neutrophil activation is implicated in the pathology of many disease processes, the in vivo mechanisms of activation of neutrophils remain relatively obscure. It has been found that there exist circulating plasma factors that lead to neutrophil upregulation in hemorrhagic shock, and this upregulation correlates with increased mortality. The nature of the circulating mediators is currently unknown.

History

Modern interest in toxic circulating factors began in the 1930's (see, Menkin et al., (1938) The American Journal of Physiology 124:524-529; Rocha et al. (1955) Histamine: Its Role in Anaphylaxis and Allergy, Springfield, Ill., Charles C. Thomas. p. 248; Knight et al. (1937) Br J Surg 25:209-26; Zweifach et al. (1957) Annals of the New York Academy of Sciences 66:1010-1021) when researchers studying shock and inflammation realized there were circulating factors that adversely affected survival and led to neutrophil activation in these conditions. In Several humoral factors involved in inflammation, including leukotaxine, leukocytosis-promoting factor, pyrexin (a polypeptide capable of inducing fever), exudin, and necrosin, each with their own fundamental inflammatory properties (Menkin (1956) Science 123:527-534) were characterizied. Leukotaxine, a polypeptide recovered from inflammatory exudate, induces neutrophil chemotaxis when injected into test animals and also promotes capillary leakage (Menkin et al., (1938) The American Journal of Physiology 124:524-529). It does not appear, however, to induce injury in cells (Menkin (1956) Science 123:527-534), implying that the neutrophil respiratory burst is not activated by this factor. In this manner, leukotaxine bears similarities with a plasma derived neutrophil chemotactic factor (Petrone et al. (1980) Proc. Natl. Acad. Sci. U.S.A. 77:1159-1163).

Another early factor is leukocytosis-promoting factor (LPF), a factor present in inflammatory exudate induces a leukocytosis (Menkin (1956) Science 123:527-534) when introduced into the circulation. Leukocytosis-promoting factor was also observed to induce hyperplasia of some of the hematopoietic cells, especially neutrophils (Menkin (1956) Science 123:527-534). This factor does not elicit injury to tissues. A third inflammatory factor, necrosin, however does result in tissue injury when injected and is a circulating factor, implicating it in tissue injury seen systemically in shock. Necrosin is thought to be responsible for inflammation and cell necrosis seen in many different inflammatory etiologies including the injurious effects seen due to ionizing radiation.

None of the factors were conclusively identified. It is probable that some, if not all of these mediators have been more recently re-identified by others and are known by different names. Despite the importance of understanding and quantifying initial neutrophil activation and the factors that lead to it in vivo, there has been surprisingly little research in this area. There are inconsistencies and discrepancies among the published reports. It is quite probable that some of the reported “factors” are in fact the same factor or are identical to other known neutrophil activators.

One of the putative neutrophil factors that has been most studied appears to be a plasma component that is activated by the free radical superoxide (O₂⁻) (Petrone et al. (1980) Proc. Natl. Acad. Sci. U.S.A. 77:1159-1163). This factor is thought to be a lipid component non-covalently bound to serum albumin that is activated to become chemotactic upon reaction with superoxide, produced via a xanthine/xanthine oxidase system. Plasma exposed to superoxide in vitro, however, was not found to stimulate neutrophil degranulation or oxidative metabolism. Superoxide dismutase (SOD) abolishes the chemotactic response when added before, but not after, exposure of the plasma to superoxide, indicating that the activity was not due to superoxide itself. Catalase, however, caused no significant reduction in chemotactic activity when added prior to xanthine oxidase, suggesting that the chemotactic factor produced was dependent specifically on the reaction with superoxide. The chemotactic activity of the factor was stable at 4° C. for 24 hours, was nondialyzable and stable for lyophilization. It is hypothesized that this factor could be an arachidonic acid metabolite such as 5-hydroxy-6,8,11,14-eicosatetranoic acid (5-HETE). It may be also be similar or identical to leukotaxine, identified some 50 years earlier (Menken et al., (1938) The American Journal of Physiology 124:524-529). The findings of Petrone et al., however, have not been duplicated by other groups that also have studied this activity.

There has also been a large body of work (see, Emerit et al. (1988) Ann Thorac Surg 45:619-624; Emerit (1994) Free Radic Biol Med 16:99-109; Emerit et al. (1991) Free Radic Biol Med 10:371-377; Emerit et al. (1995) Free Radic Biol Med 15:405-415; Emerit et al. (1997) Dermatology 194:140-146; and Emerit et al. (1996) Proc. Natl. Acad. Sci. U.S.A. 93:12799-12804) directed “clastogenic” factors, which were originally discovered in patients exposed to high levels of radiation, and named because of their ability to break chromosomes (Emerit et al. (1990) Methods Enzymol. 186:555-564). Some of these factors also induce neutrophil chemiluminescence using the probe luminol, presumably through the production of superoxide or other reactive intermediates (Emerit et al. (1995) Free Radic Biol Med 15:405-415). Clastogenic factors in plasma stimulate naive neutrophils in vitro (Emerit et al. (1990) Methods Enzymol. 186:555-564). Necrosin (Menkin (1956) Science 123:527-534) may be among these factors. Clastogenic factors have been found to be produced clinically by as little as thirty-eight minutes of cardiac and lower-body ischemia during aortic clamping (Fabiani et al. (1993) Eur. Heart J.: 12-17).

Not all clastogenic factors are neutrophil activators nor are all neutrophil activators clastogenic. Clastogenic factors are not produced in plasma in the absence of cells, suggesting that they are the products of cellular disruption by the superoxide radical. Among the clastogenic factors identified are hydroxynonenal, a lipid peroxidation end product, tumor necrosis factor-alpha (TNF-α), and inosine triphosphate (ITP). The presence of each of these factors depends in part on the disease condition studied. Clastogenic factors in the plasma of patients after cardiac ischemia are currently of unknown origin.

Another unknown neutrophil chemotactic factor, known as Nourin-1 (Elgebaly et al. (1993) Circulation 88:1-240), appears in plasma after coronary artery occlusion and is thought to be produced by superoxide. It is chemotactic towards neutrophils and stimulates neutrophil activation. This factor is of peptide composition, degradable by proteases but not the product of a larger protein cleavage (Elgebaly et al. (1989) J. Mol. Cell Cardiol 21:585-593; Elgebaly et al. (1992) J Thorac Cardiovasc Surg 103:952-959). Nourin-1 is water soluble and is produced by a number of tissues, including vascular smooth muscle, endothelium and in cornea, stomach and coronary arteries.

Recently, the finding of neutrophil activating factors in plasma after ischemic events has been confirmed (Peterson et al. (1993) Ann Vasc Surg 7:68-75; Silliman et al. (1997) Transfusion 37:719-726; and Barry et al. (1997) Endovasc Surg 13:381-387). These factors appear within minutes after the start of reperfusion and, thus, may be related to superoxide generation One component may be platelet activating factor (PAF) or PAF-like substances, since production of neutrophil activators by reperfusion plasma was diminished by application of the PAF inhibitor WEB 2170 (Silliman et al. (1997) Transfusion 37:719-726).

Another, apparently different, set of neutrophil activating factors (Pfister et al. (1996) invest Ophthalmol Vis Sci 37:230-237; Pfister et al. (1993) invest Ophthalmol Vis Sci 34:2297-2304) that can separated from plasma by centrifugation at 15,000 G and from neutrophils subjected to treatment with N NaOH has been identified as the tripeptide having the sequence N-acetyl-Pro-Gly-Pro (312 MW) or N-methyl-Pro-Gly-Pro (Pfister et al. (1995) invest Ophthalmol Vis Sci 36:1306-1316). These factors are long-lived and can circulate throughout the body.

Cellular Activation

Activated neutrophils release a number of toxic substances including free radicals, proteases and their products that kill cells and ultimately destroy tissues. Neutrophils also release cytokines and other inflammatory substances, resulting in the recruitment of additional neutrophils and activated cells, further propagating inflammation and injury. In the case of bacterial infection, this activation can be beneficial, destroying foreign pathogens that would otherwise be deleterious to the host. If uncontrolled, however, the effects of cell activation can be extremely destructive and even lethal.

Many factors modulate neutrophil upregulation, including physical stimuli, such as shear stress (Moazzam et al. (1997) Proc. Natl. Acad. Sci. U.S.A. 94:5338-5343 ), and a host of chemical mediators (Ferrante (1992) Immunol Ser 57:499-521). A great number of both types of neutrophil activating factors have been identified in vitro. Chemical factors can be broadly grouped into one of two categories: receptor mediated and non-receptor mediated. Non-receptor mediated neutrophil activating factors, such as Phorbol 12-myristate 13-acetate (PMA), tend to be generally non-specific compounds such as petroleum derivatives or detergents and are ubiquitous in number (Wjentes et al. (1995) Semin Cell Biol 6:357-365). Receptor mediated factors are specific activators for neutrophils and include, the bacterial peptide formyl-methionyl-leucyl-phenylalanine (fMLP) and platelet activating factor (PAF).

Measurements of neutrophil activating factors have been made in vivo as well as in vitro and are reported in many different pathological states, including shock (Barroso-Aranda et al. (1992) Circ Shock 36:185-190), arthritis (Downey et al. (1995) Semin Cell Biol 6:345-356), myocardial infarction (Shandelya et al. (1993) Circulation 87:536-546), pancreatitis (Sandoval et al. (1996) Gastrenterology 111:1081-1091), and sepsis (Yoshikawa et al. (1990) Methods Enzymol 186:660-665). Among the activators consistently identified are PAF (Graham et al. (1994) J Lipid Meidat Cell Signal 9:167-182), tumor necrosis factor-α (TNF-α) (Caty et al. (1990) Ann Surg 212:694-700), interleukins Il-1 and Il-8 (Ferrante (1992) Immunol Ser 57:499-521), bradykinin (Hoffman et al. (1997) Microsc Res tech 37:557-571), and LTB₄ (leukotriene B₄) (Letts (1987) Cardiovasc Clin 18:101-113, as well as other arachidonic acid degradation products (Langholz et al. (1990) Prostaglandins Leukot Essent Fatty Acids 39:227-229).

In none of these cases has any more than a relationship to neutrophil activation been identified. Measurements are typically made before and after a designated insult, such as infarction, shock, sepsis, and others, and the plasma levels of the activators of interest are compared. Although these types of experiments provide information about the nature and magnitude of different activators, the role that each mediator plays in neutrophil activation is usually unknown. After an initial stimulus in which a neutrophil population is activated, feed-forward upregulation of not only neutrophils but other cell types results in the production of numerous inflammatory products. Therefore the presence of numerous activators in the circulation is indicative of systemic upregulation in general and is not necessarily due to one of the measured factors per se.

While serine proteases are not particularly stimulatory towards neutrophils, serine proteases have been found to produce activating factors in organs that otherwise are not excitatory towards neutrophils Neutrophil activation by the pancreatic homogenate has been found to be inhibited by protease inhibitors. These factors are released during shock and contribute to the lethality and morbidity seen in different pathologies as well as more benign and outwardly healthy conditions. Recognition and understanding of the mechanisms for the release of these factors as well as their identification should aid in the treatment, not only of shock, but of chronic conditions where inappropriate neutrophil upregulation has been identified.

Thus, it is known that cells in microcirculation can be encountered in a relatively quiescent state and in various stages of activation. Cellular activation is a normal physiological response that is essential for survival from infection. There is evidence, however, that cardiovascular complications, such as myocardial infarction, venous ulceration and ischæmia/reperfusion injury may be associated with an activation of cells in circulation. The underlying stimuli and reasons therefor are unknown. The evidence that implicates activated cells in pathogenesis of microvascular disorders makes identification of the source(s) and causes of activation of key importance.

Therefore, it is an object herein, to identify factor(s) that are responsible for cellular activation. It is also an object herein to use such factors to aid in understanding the underlying processes and to serve as targets for diagnosis of disease and for diagnostic intervention.

It is also an object herein to provide a means to improve outcomes in cardiovascular, inflammatory diseases and other disorders and conditions. It is also an object herein to provide methods for identifying drug candidates for treatment of such disorders and conditions.

### SUMMARY OF THE INVENTION

Diagnostic methods that rely on the use of one or more assays that assess cellular activation are provided. The assays are performed on whole blood or leukocytes, and indicate singly or in combination the level of cardiovascular cell activation, which is pivotal in many chronic and acute disease states. Cardiovascular cell activation is pivotal in many chronic and acute disease states by initiating or contributing thereto. The level of cell activation will be statistically correlated with disease states.

The activation status of neutrophils and other inflammatory cells is of central importance in not only disease states, such as ischemia, infection, trauma, inflammatory diseases, but also to ‘healthy’ individuals. As shown herein such cellular activation, particularly neutrophil activation, can be used as an indicator of therapeutic outcome and also as therapeutic target. A method of indicating therapeutic outcome by assessing the state of activation of such cells is provided herein. The cellular activation may be assessed by any assays known to those of skill in the art, such as those exemplified herein, that are used to measure cellular activation. For example, cell activation may be assessed superoxide production, such as as defined by the nitroblue tetrazolium test and lucigenin-enhanced chemiluminescence, and/or actin polymerization, such as defined by the pseudopod formation test, are indicators of cellular activation levels.

Assays are performed on whole blood or leukocytes and indicate, individually or in combination the level of cardiovascular cell activation. The results of the assays can be used within a clinical framework to support therapeutic decisions, including but not limited to: further testing for infectious agents; anti-oxidant or anti-adhesion therapy; postponement and optimal re-scheduling of high-risk surgeries; classifying susceptibility to and progression rates of chronic disease such as diabetes, atherogenesis, and venous insufficiency; extreme interventions in trauma cases of particularly high risk; and activation-lowering therapies as yet to be developed.

The results of specific cell activation assays are used in guiding therapeutic decisions such as, but not limited to: further testing for infectious agents, anti-oxidant or anti-adhesion therapy, postponement and optimal re-scheduling of high-risk surgeries, classifying susceptibility to and progression rates of chronic disease such as diabetes, atherogenesis, and venous insufficiency; extreme interventions in trauma cases of particularly high risk and activation-lowering therapies.

Methods of assessing treatment options and methods of treatment are also provided in which cellular activation is measured, and, if elevated, activation lowering therapy is administered prior to further treatment. Activation lowering therapy methods include any method that lowers activation, including alterations in lifestyle, including stress management, exercise and diet, administration of drugs, such as heart medications, aspirin, administration of protease inhibitors, including Futhan (nafamostat mesilate, which is 6-amidino-2-naphthyl p-guanidino-benzoate dimethanesulfonate), as described herein.

Methods for diagnosis based upon these assays are also provided. One or more of these assays alone or in combination will be related to disease outcomes and can be used to support useful therapeutic decisions. The resulting diagnostic methods improve treatment, outcome and, will also reduce per-patient costs.

Also provided is as composition derived from a pancreatic homogenate that contains cell activating factors, which can serve as targets for drug screening to identify drug candidates for use in activation lowering therapies. The composition, which contains neutrophil activating factor(s) found in the pancreas, activates cells in vitro and in vivo, and can be used to screen for factors that inhibit activation. In such assays the cells, particularly cells subject to activation, such as PMN and endothelial cells, are contacted with the homogenate either in the presence of a test compound or before addition of the compound or after addition of the test compound. The level of activation of the cells is then assessed and compared to a control, typically the same experiment performed either in the absence of the test compound and/or in the presence of a known activator, such as PAF. Compounds that inhibit activation are selected as candidates for drugs that can be used to block or inhibit cellular activation.

Compositions containing the pancreatic homogenate or active fractions, particularly active fractions containing active compounds of molecular weights less then about 3 kD are also provided.

Methods of treatment of disorders and condition related to inappropriate or chronic cell activation are provided. In particular, treatment by administration of effective amounts of compounds that lower cell activation. Such compounds include agents known to lower cell activation, including aspirin, also new compounds that inhibit the activation factors in the pancreatic homogenate, and also enzyme inhibitors, such as protease inhibitor. Compositions containing broad protease inhibitors, particularly serine protease inhibitors, and methods of treatment using the compositions are provided. In a preferred embodiment, the protease inhibitor is Futhan (nafamostat mesilate, which is 6-amidino-2-naphthyl p-guanidinobenzoate dimethanesulfonate) and treatment with a pharmaceutical composition containing an effective amount of Futhan is contemplated.

Thus, methods in which Futhan or a similarly broad protease inhibitor to treat patients in shock, suffering trauma or otherwise having compromised (i.e. individuals with activated circulating neutrophils) systems in order to minimize vessel/tissue injury are provided. Administration is contemplated as soon as possible in the instance of a trauma or immediately prior to surgery or invasive clinical procedure in the case of compromised patients.

A drug screening assay for identifying compounds that inhibit or lower the level of cellular activation is also provided herein. Assays for identifying activation factors in tissues are also provided.

Also provided is a method for enriching patient populations for clinical trials to by testing patients for cellular activation levels, and excluding those with high levels (about one standard deviation above the mean or other selected cut-off) from the clinical population. This will eliminate those patients whose outcomes will be unfavorable regardless of therapy, and thereby provide a means to better assess efficacy of a clinical protocol or treatment.

Also provided are articles of manufacture that include packaging material and a pharmaceutical composition containing a protease inhibitor, contained within the packaging material, where the pharmaceutical composition is effective for lowering cell activation levels or preventing increased cell activation, and the packaging material includes a label that indicates that the pharmaceutical composition is used for lowering cell activation levels. The label may also indicate disorders for which cell activation therapy is warranted.

### DESCRIPTION OF FIGURES

FIG. 1 depicts a summary of the relation of cell activation to disease showing that cardiovascular cell activation plays a central role in cardiovascular diseases and immune response and that it: responds to lifestyle factors, as well as trauma, ischemia, infection; initiates or potentiates atherosclerosis; causes poor outcome in trauma, shock, MI; participates in a disease positive feedback loop; and is governed by circulating plasma factors;

FIG. 2 schematically depicts cell activation diagnostic and therapy points (ARDS refers to Adult Respiratory Distress Syndrome, and MOF refers Multiple Organ Failure.

FIG. 3 shows potential therapeutic intervention points; 3a) depicts intervention downstream of activation, such targets include integrin IIa/IIIb for platelet aggregation, VLA-4 for T-cells and eosinophils, CD-18 for neutrophil adhesion, ICAM-1 for endothelial adhesion, selections E, P for neutrophil migration; b) intervention before activation by attacking activating factors as proposed herein;

FIG. 4 presents chemical formulae of several proposed PAF-like factors (Itabe et al. (1988) Biochim Biophys Acta 145:415-425, Englberger et al. (1987) International J Immunopharmacy 9:275-282; and Tanaka et al. (1993) Lab Invest 70:684-695), the last set of PAF-like factors with variable sn-2 side chains are from bovine brain and may be similar to activating factors found in the pancreatic homogenate provided herein;

FIGS. 5a-5c present a list of peptides tested in the computer program described herein, with a letter indicating the species origin of the peptide, followed by a brief description of the peptide or its believed mechanism of action. Letter Key for peptide origin: b=bovine; h=hamster; m=man; o=other; r=rat. The peptides were compared with the sizes generated in the mass spectra analyses of the pancreatic homogenate provided herein.

### DETAILED DESCRIPTION OF PREFERRED EMBODIMENTS

A. Definitions

Unless defined otherwise, all technical and scientific terms used herein have the same meaning as is commonly understood by one of skill in the art to which this invention belongs. All patents and publications referred to herein are incorporated by reference.

As used herein, cell activation refers to changes in and interactions among circulating white blood cells, including leukocytes, cells lining blood vessels, including endothelial cells, and platelets. These changes are evidenced by increased “stickiness” of cells, changes in shapes of cells, free radical production and release of inflammatory mediators and enzymes. Activated cells project large pseudopods, and express adhesion molecules on their surfaces. For example, adhesion molecules and villi attache macrophage and monocytes to endothelium. Macrophage and monocytes may then infiltrate into tissue outside the blood vessel beginning the development of atherosclerosis, venous insufficiency ulcers an diabetic retinopathy.

Cell activation is necessary for normal human immune defense mechanisms, but inappropriate or excessive activation leads to or participates or intensifies many diseases, including, but not limited to: arthritis, atherosclerosis, acute cardiovascular incidents, Alzheimer's Disease, hypertension, diabetes, venous insufficiency, autoimmune disease and others. Cell activation is a major contributor to rejections processess in organ transplants, and to predisposition to poor outcomes in trauma and high risk surgeries.

For example, LPS (lipopolysaccharide) binds to immunoglobin M and this complex activates the complement system with the release of C3b, which material in turn activates the polymorphonuclear leukocytes (PMN), monocytes, neutrophils, macrophage and endothelial cells. The activation of these substances stimulates the release of several mediators of septic shock including tumor necrosis factor (TNF-α) interleukin-1 (IL-1) and other interleukins including IL6 and IL-8, platelet-activating factor (PAF), prostaglandins and leukotrienes (see, eg., (1991) Ann. Intern. Med. 115:464-466 for a more comprehensive listing). Of these, the two cytokines TNF-α and IL-1 lead to many of the physiologic changes which eventuate into septic shock.

The LPS-stimulated macrophages also release other free-radicals, including oxyfree-radicals from arachidonic acid metabolism, which free-radicals can also cause extensive damage to endothelial cells. These lead to aggregation and circulatory collapse, which in turn leads to hypotension, tissue damage, multi-organ failure and death. Thus, excess production of the above mentioned free-radicals is linked to the mortality associated with septic shock.

As used herein, polymorphonuclear leukocytes (PMNs). Polymorphonuclear neutrophil granulocytes (PMN) are cells which are mobilized during inflammatory phenomena and which can be stimulated by various compounds, such as, for example, formylmethionyl-leucyl-phenylalanine (FMLP) or prostaglandins E (PGE1). The PMN granulocytes respond to these extracellular stimuli with an activation of the oxygen metabolism with release of toxic oxygenated metabolites. An excessive response of the PMN granulocytes may be the cause of a painful inflammation and is also accompanied by a reduction in the level of cyclic adenosine monophosphate (cAMP) in these granulocytes.

The term “migration” with respect to PMN is meant to include the adhesion of PMN to the epithelium and the complete traversion across the epithelium to the other side. Activation of leukocytes, such as MNs and monocytes, and their migration to sites of inflammation appear to take place in vivo as a result of an inflammatory response. Under normal circumstances, PMN rarely adhere to the epithelial surface, and thus such adhesion is considered the rate-limiting step in the migratory process.

Activated PMNs, among other mediators, cause the formation of oxygen-containing free-radicals. These free-radicals are produced as part of the body's defense against the invasion of foreign organisms and their toxic products. PMN specifically generates the superoxide anion radical (O₂⁻). This free-radical when acted upon by the enzyme superoxide dismutase (SOD) forms hydrogen peroxide. Excess hydrogen peroxide in the presence of iron generates a second oxygen-containing free-radical, the hydroxyl free-radical. In addition, activated neutrophils can generate oxyradicals by stimulating the NADPH oxireductase reaction. The release by neutrophils of oxyfree-radicals and proteases causes extensive damage to endothelial cells. In addition, adhesion of activated neutrophils to endothelial cells leads to vascular permeability, which in turn causes much of the damage associated with septicemia and septic shock.

As used herein, treatment means any manner in which the symptoms of a conditions, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein an effective amount of a compound or composition for treating a particular disease is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Typically, repeated administration is required to achieve the desired amelioration of symptoms.

As used herein, activation lowering therapy (A.L.T.) refers to any means in which the level of activated cells is lowered. Such means include lifestyle and dietary changes, drug therapy, such as aspirin, pentoxifylline, Daflon 500 (a flavenoid), anti-inflammatories, inderal, heparin, coumadin, Futhan and other protease inhibitors.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

B. Cellular Activation and Disease

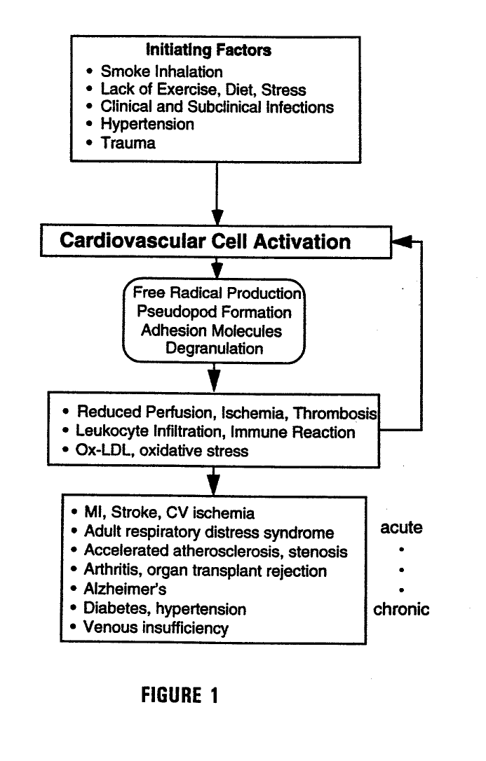

The activation of cells in the cardiovascular system is linked to acute and long term complications (see, FIG. 1). Among the cells that play a primary role are endothelial cells, vascular smooth muscle, and the circulating cells (erythrocytes, platelets, leukocytes). These cells can be encountered in a relatively quiescent state, a condition that is associated with a low level of cardiovascular complications as well as lower response after a cardiovascular challenge, and they can be encountered in a more activated state that is associated with cardiovascular complications. The activated state involves among other things production of free radicals and changes in cell morphology and elasticity, which can increase adhesion and decrease capillary flow. Such changes are part of the normal responses to infection. If inappropriately or chronically present, they can initiate or contribute to disease states,including myocardial infarction, hemorrhagic shock, diabetes, diabetes, hypertension, and venous insufficiency.

Myocardial Infarction (MI) and Stroke (CI):

Reduced flow, increased free radical generation, and increased adhesion are believed to contribute to atherogenesis, stenosis and ultimately thrombosis via multiple mechanisms. Free radicals increase the production of oxidized low density lipoproteins (Ox-LDLs) (Steinberg, (1997) “A critical look at the evidence for the oxidation of LDL in atherogenesis,” Atherosclerosis) and permeability of the endothelium, both of which are believed to lead to monocyte infiltration and plaque formation (Lehr et al. (1992) Arteriosclerosis and Thrombosis 12:824-829. Reduced flow increases the extent of adhesion of leukocytes to endothelium mechanically via encounter time and decreased shear, as well as through activation of the leukocytes with an associated up-regulation of adhesion molecule expression and spontaneous shape changes.

During and after MI or CI events, the associated ischemia and reperfusion produce free radicals and activate leukocytes (Chang et al. (1992) Biorheology 29:549-561; Grau et al. (1992) Stroke 23(1):33-39)increasing the chances of adhesion and permanent blockage of microcirculation, with consequent tissue damage (Schmid-Schonbein et al. (1986) The American Journal of Cardiovascular Pathology 1(1):15-30; Welbourn et al. Circulation Research 71(1):82-86; Petrasek et al. (1996) Am. J. Physiology H1515-H1520; Jerome et al. (1993) Am. J. Physiology H479-H483; Garcia et al. (1994) Am. J. Pathology 144(1):188-198). Decreased cerebral perfusion and possibly also increased permeability of the blood-brain barrier are associated with progressive dementia.

Hemorrhagic Shock

Hemorrhagic shock was first studied in depth during and after the First World War. Following this period, major progress in defining shock and quantifying the lethal effects of global hypotension was made (see, Wiggers (1995) Physiology of Shock, 1st Ed, Commonwealth Fund, NY, N.Y.). The Wiggers' shock model, in particular, arose as a result of difficulties in the standardization of various shock protocols. By selecting a shock protocol with a step-wise systematic decrease in central blood pressure, usually to 40-60 mmHg, reproducible results in anesthetized and awake animals could be achieved.

Global ischemia and subsequent reperfusion lead to complications that are accompanied by cell and organ damage. Tissue damage after hemorrhagic shock depends on the degree of pressure reduction, the choice of anesthesia (if applicable), as well as duration of ischemia and the nature of the organs affected. Some organs, notably skeletal muscle, may survive periods of up to four hours of ischemia without adverse effects. Others, such as those in the splanchnic region and brain, are more sensitive and do not tolerate low-flow states for an extended length of time. Organs such as the heart can tolerate limited ischemia for short durations.

There is a time window in which reperfusion is desirable and clinically possibly relevant, since not all cells are killed and salvage may be possible. Interventions against ischemia-reperfusion, including hemorrhagic shock must be made during the ‘treatment window’ or before when tissue is still salvageable. After this time, injury is irreversible regardless of intervention (Sussman et al. (1990) Methods Enzymol 186:711-783). Shorter durations of ischemia followed by reperfusion result in less impairment of tissue function, while longer periods of ischemia may lead to cell death and tissue necrosis, whether or not there is reperfusion. Total occlusion of a vessel, as opposed to low flow states, leads to predominantly anoxic cell death rather than free radical interactions when reperfusion is not obtained (McCord (1986) Adv. Free Rad Bio & Med 2:325-345).

In hemorrhagic shock as well as ischemic states in general, the decrease in blood flow results in reduced oxygen transport to tissue as well as impaired waste product removal. These factors lead to impaired function and eventually death of the tissue. Paradoxically, the replacement of shed blood in the case of hemorrhagic shock, or the re-establishment blood flow to previously ischemic tissue leads to the phenomenon known as “reperfusion injury.” This injury, appears to be due to the reoxygenation of previously ischemic tissue and production of oxygen free radicals and other toxic substances. Free radicals, molecules with an unpaired electron, are highly reactive and are known to cause tissue damage due to breakdown of cell membranes, denaturing of proteins and destruction of nucleic acids. Although oxygen free radicals have been implicated in reperfusion injury, which free radicals are involved and their site of production has not been resolved. The prevailing hypothesis holds that hypoxia caused by low blood flow and subsequent oxygen exchange in ischemic tissue leads to activation and upregulation of otherwise benign enzymes and production of free radical species in larger amounts.

Massive ischemia and reperfusion (I/R) in animals is much more likely to lead to death when the animals have prior or concurrent high levels of cell activation (Barroso-Aranda et al. (1989) Am. J. Physiological Soc H846-H852). The observed relationship between likelihood of death and levels of cell activation coupled with the activation in response to I/R events suggests that extremely high levels of activation after trauma or hemmorhagic shock is an indicator of particularly high risk in these critical care situations. For example, cell activation probably leads to ARDS (adult respiratory distress syndrome) and MOF (multiple organ failure) in which activated white cells clog up the capillary beds of the lungs (ARDS) and clog up the capillary beds of other organs (em, liver, kidneys, pancreas). Similarly, cell activation resulting from massive infection appears to be a major contributor to death in septic shock. Thus, based on patients' levels of cell activation vascular surgeries and other invasive procedures can be postponed. Activation lowering therapy(ies) can be instigated.

General Health and Subclinical Compromise Thereof

Good perfusion is generally associated with health and well being. Normal blood flow lowers the activation levels of leukocytes via shear stress (Moazzam et al. (1997) Proc. Natl. Acad. Sci. U.S.A. 94:5338-5343). High levels of activation are associated with infections and cardiovascular complications (see, Mazzoni et al. (1996) Cardiovascular Research 32:709-719) It is reasonable therefor to expect that subclinical compromise of health is closely related to inappropriate activation levels, and that subclinical disorders, such as subclinical infections, will be indicated by elevated activation levels.

Diabetes, Hypertension, and Venous Insufficiency

It is very likely that activation of cells in the cardiovascular system, such as leukocytes or endothelial cells, accelerates diabetic retinopathy (Schroder et al. (1991) Am. J. Pathology 139(1):81-100) and venous insufficiency disease (Edwards et al. (1994) “White blood cell distribution in chronic venous insufficiency”, Chapter 7 of Microcirculation in Venous Disease, Smith, Ed.), and likely that it accelerates development of diabetes via free radical damage to pancreatic B-cells (Schroder et al. (1991) Am. J. Pathology 139(1):81-100), either mediated by hyperglycemia or independent of it.

Activation levels, especially free radicals and bioactive lipids, also may mediate hypertension (Sagar et al. (1992) Molecular and Cellular Biochemistry 111:103-108; Shen et al. (1995) Biochem. Cell Biol. 73:491-500; Schmid-Schonbein et al. (1991) Biochem. Cell Biol. 17(3):323-330).

C. Cell Activation Diagnostic and Therapy Points

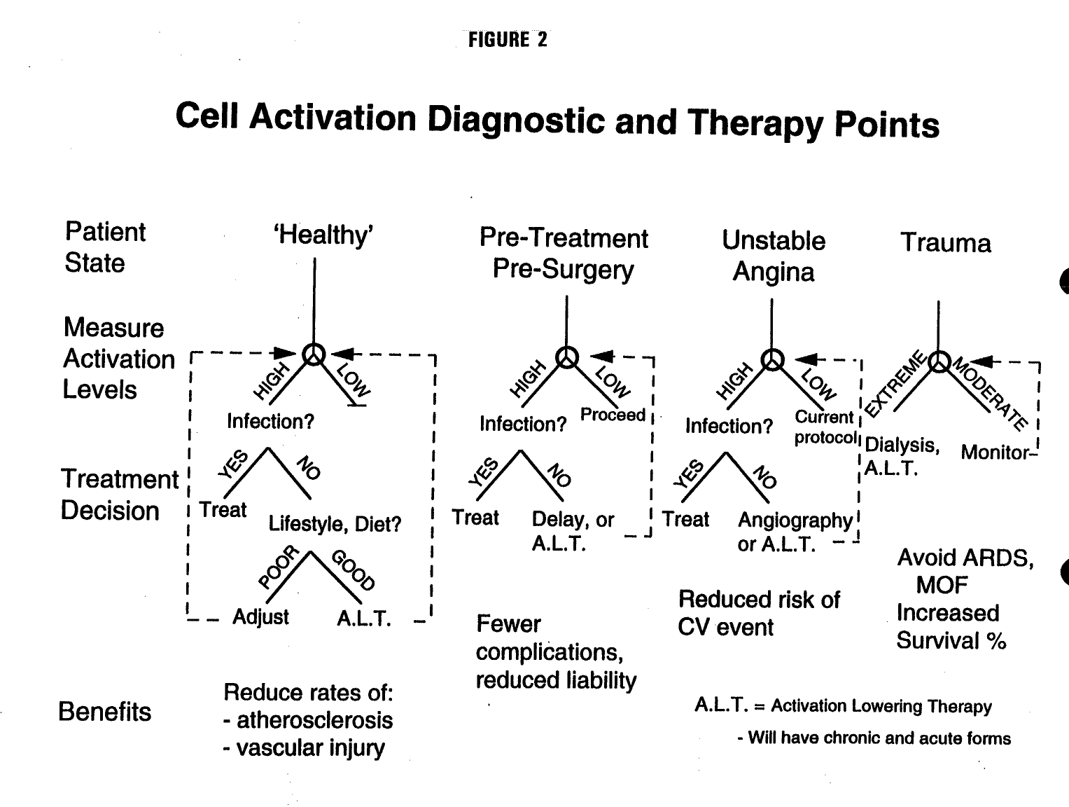

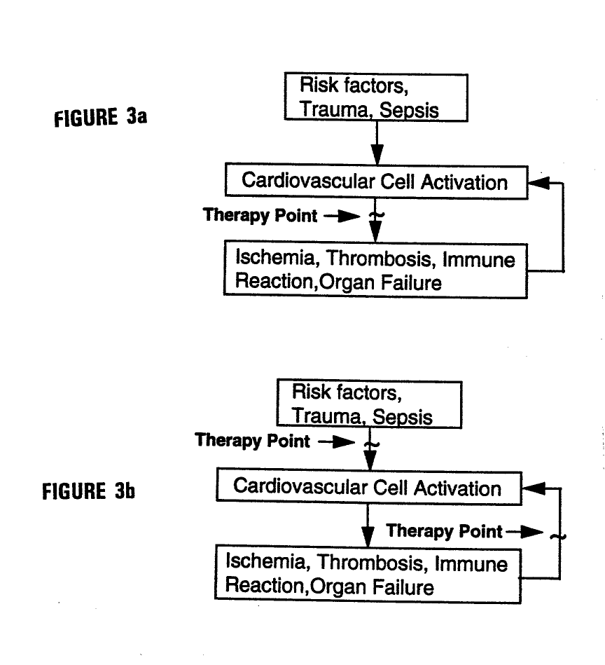

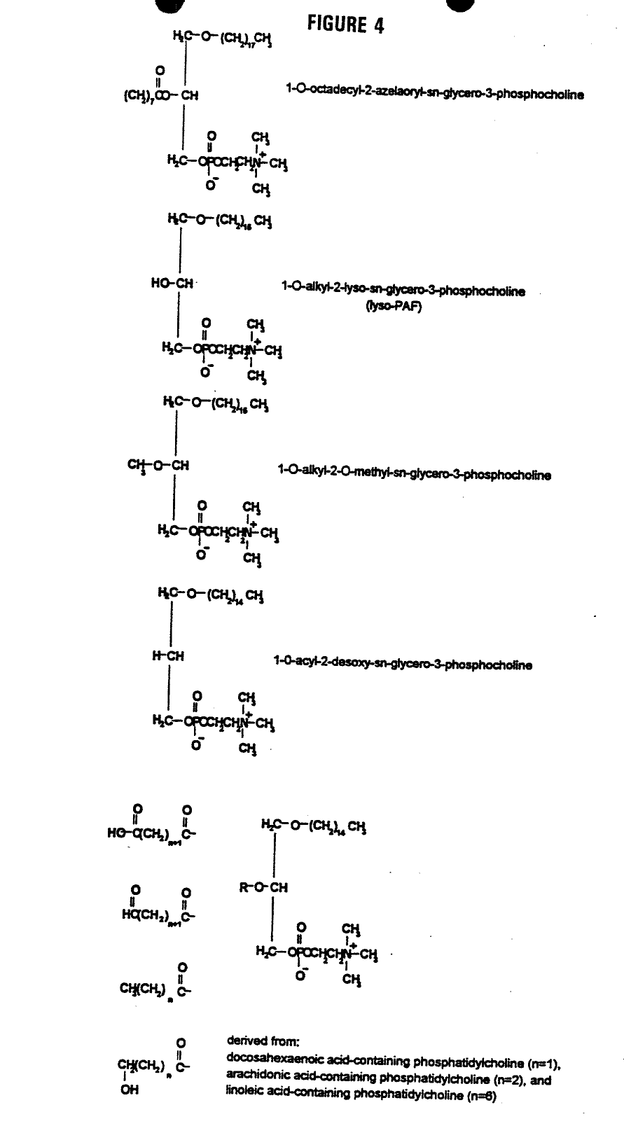

The use of cell activation for diagnosis and therapeutic intervention is shown FIG. 2, which sets forth the paradigm for the methods of assessing treatment options provided herein. Since activation is pivotal in disease outcomes, trauma outcomes, and general long term good health, measurement of activation levels should be performed in healthy individuals who present no disorders. Identification of healthy individuals with elevated levels of activated cells, permits early identification of at-risk individuals and permits early intervention, in chronic and also in acute diseases. As shown in FIG. 2, in a seemingly healthy patient activation levels are measured. If low, then no treatment or changes in lifestyle are recommended. If the levels are elevated (above the 50th percentile, more likely above the 20th percentile, or one standard deviation above the mean or more), then tests to determine the presence of subclinical infection or other cell activating condition are performed. If those tests are negative, then lifestyle and diet should be examined, and if, necessary, modified. If diet is good, and lifestyle is generally good and stress-free, then activating lowering therapy can be instituted.

Testing cell activation levels pre-surgey, particulaly elective surgery, then the levels can be used to assess the likely of compliations from surgery and organ transplant rejection. If high levels of cell activation that are not the result of infection are found, then surgery should be postponed. Activation lowering therapy considered. Similarly, in unstable angina, the levels of cell activation are indicative of the risk of a cardiovascular event. Thus, if levels are high, activation lowering therapy and/or more aggressive treatment should be pursued. In trauma situations, the level of cell activation can aid in selecting treatment protcol and timing thereof. High levels of activation are associated with ARDS and MOF in the emergency room. Activation lowering therapy should reduce the risk thereof.

Thus, in general, if a high level of cell activation is observed, then activation lowering therapy should be adminstered prior to further treatment. Activation lowering therapy includes adminstration of known pharmaceuticals, such as aspirin and cardiovascular medications, dialysis and other such treatments. As shown herein, protease inhbitors, particularly serine proteases, such as Futhan, can be administered. It is also contemplated herein, that compounds identified using the methods herein for such identification will be administered.

Cellular activation will be statistically correlated with disease states. It is considered elevated when is is above the normal range, which can be established by sampling “healthy” people and determining the mean. In particular, individuals with activated cells in the upper 20% of levels or one standard deviation above the mean are considered candidates for activation lowering therapy.

Tests for Detecting Cell Activation

In practicing the method, one or more tests for cell activation would be performed. Thes tests, discussed and exemplified below in more detail below and include tests that assess indicators of activation, such as changes in shape and free radical production. For example cell morphological changes may be quantified with direct microscopic examination, with or without fluorescent staining of F-Actin filaments present in pseudopods, or with fluorescence activated cell sorting techniques. Superoxide anion production can be detected and quantified using chemiluminescence generating reagents, such as luminol, isoluminal and lucigenin, that quantitatively react therewith. Free radicals can be assessed by NBT (nitroblue tetrazolium). Adhesion can be assessed by various immunassays that detect surface adhesion molecules, such as CD11, CD18 and L-selectin and others. Other indicators of activation include expression of certain factors, such as interleukin and TNF-α, which can be measured by known immunoassays.

Activation can also be assessed by sampling plasma and determing whether it activates cells, such as endothelial cell cultures. Plasma can be tested for clastogenic activity by standard methods. Although there is a high correlation between the different cell activation assay measures, it is likely that there will be different combinations of indicators which are most informative in any situation. For example, plasma activator levels might be high but circulating activated neutrophil counts low due to sequestration of the activated cells in the microcirculation. Also, genetic, age, and environmental differences between patients will complicate the interpretation of the assays. Clinical tests are in preparation to relate statistically cell activation measures to disease outcomes, to find the formulas which are invariant to patient differences, and to establish the best predictive procedures and activation lowering therapies in different situations. The measurement of cell activation and circulating plasma factors also serves as an effective tool to evaluate the effectiveness of new interventions prior to execution of full-scale clinical trials. Drug candidates thereby may be rejected, or patient populations enriched for more favorable response to the candidate drug.

D. Pancreatic Neutrophil Activating Composition

Factors circulating in rat shock plasma will activate naive neutrophils in vitro (see, em., Mazzoni et al. (1996) Cardiovasc Res. 32:709-719; Barroso-Aranda et al. (1989) Am J Physiol:H846-852; Barroso-Aranda et al. (1992) Circ Shock 36:185-190; Barroso-Aranda et al. (1989) Am J Physiol:H415-421; and Shen et al. (1990) Circulatory Shock 31:343-344). These studies also showed that the level of neutrophil activation in vitro induced by plasma taken before a shock protocol corresponds inversely with that animal's survival in shock, giving rise to the idea of ‘preactivation’ . Neutrophil levels of preactivation (‘resting’ neutrophil activation before shock) correlate with lipid peroxidation production in hemorrhagic shock experiments. The time course of lipid peroxidation results also matches that of plasma peroxide in hemorrhagic shock, but it is unclear how the two measurements are related. The possibility exists that oxidation of lipid membranes leads to the increase seen in plasma peroxides.

Hemorrhagic shock is a globally systemic insult and does not provide information as to the possible origin of these factors. A rat splanchnic arterial occlusion (SAO) shock model was studied. Previous work (see, Lefer et al. (1970) Circ Res 26:59-69) had shown that a myocardial depressant factor (MDF) is produced during hemorrhagic shock. Production of MDF is enhanced in the SAO shock model due to a more complete ischemia and subsequent autolysis of the pancreas than in hemorrhagic shock. It was hypothesized that MDF could be identical or perhaps co-localized with the in vivo neutrophil activating factors measured in hemorrhagic shock. SAO shock was found to result in the release of plasma factors that activate neutrophils in vitro, implicating the site of the production of neutrophil-activating plasma factors as the splanchnic region.

The finding of plasma-derived neutrophil activating factors after SAO shock indicated that the splanchnic region is a possible site for the formation of these factors. To study this possibility, rat homogenates were made of the splanchnic organs as well as other representative viscera. Liver, intestine, heart, spleen, pancreas, adrenal and kidney tissues were homogenized and measured for neutrophil activating properties in vitro, before and after incubation of the homogenate at 38° C. for 2.5 hours to maximally stimulate any enzymatic degradation processes that might be necessary to produce such a factor. Of the tissues measured, only pancreas homogenate stimulated neutrophils to a significant extent. Neutrophil activating factors were also found in the pancreatic homogenate of the pig, indicating that the pancreatic activating factors are not species specific. Incubated pancreatic homogenate activated neutrophils to a greater extent than non-incubated samples, but non-incubated pancreatic homogenate was still significantly stimulatory towards naive neutrophils. These findings demonstrate that the pancreas is the only tissue of the organs measured that contains appreciable amounts of a neutrophil activating factor as determined in vitro and suggests that such a factor is already preformed. In contrast, MDF activity is non-existent in unincubated pancreatic homogenates, indicating an enzymatic step necessary for its production. The enhancement of neutrophil activation seen in incubated homogenate may reflect increased lysosomal degradation or cell lysis necessary for maximal release of the activator.

The activating factors, found in the pancreas do not appear to be protease in nature, as direct incubation of neutrophils with trypsin and chymotrypsin do not activate neutrophils. Preliminary isolation of pancreatic homogenates suggests there exist a number of activating factors produced in the pancreas, including a series of low-molecular (<3 kD) weight activators that may include platelet activating factor-like (PAF-like) substances. Further studies must to conducted to determine the definitive nature of these activators.

Further experiments to determine the origin of these factors were designed to determine the in vitro ability of rat tissue homogenates to activate neutrophils. Of the tissues tested, only the pancreas possesses the ability to activate naive neutrophils in vitro. As demonstrated herein, the pancreas appears to be a source of the circulating plasma factor(s) in hemmorhagic shock that activate naive neutrophils and appear to lead to myocardial suppression, multi-organ failure and death in animal models. The pancreatic cell-activating factor appears to be of low-molecular weight (<3000 Da).

As shown herein, when incubated with homogenates of other organs, the pancreatic homogenate supernatant, and also trypsin and chymotrypsin, cause cell-activating factors to be released from these other homogenates. Serine protease inhibitors, such as FUTHAN, inhibit production of the cell activating factors in in vitro experiments and reduce systemic responses in vivo. These observations and others indicate that the pancreas is the source of an endogenous protease, which cleaves active fragments from pancreatic and non-pancreatic proteins.

Subsequent experiments showed that other tissues could be made excitatory towards neutrophils by the addition of limited concentrations of pancreatic homogenate or serine proteases. Protease inhibitors, in particular the serine protease inhibitor Futhan (nafamstat mesilate; a nonpeptidyl low molecular weight protease inhibitor 6-amidino-2-naphthy-p-guanidinobenzoate dimethanesulfonate; see, Fuji et al. (1981) Biochim. Biophys. Acta 661:342), mitigate neutrophil activation in vitro and mortality in animals subjected to either SAO shock or injected with pancreatic homogenate.

Experimental Results—Synopsis

The results from the experiments done to identify and characterize and in vivo neutrophil activating factor are summarized in depth in each Example. Some of the main points are highlighted here for review.

Provided herein is a composition, a partially purified pancreatic homogenate, that contains factors that activate cells, including neutrophils. The composition contains factors that include a low-molecular weight component (<3 kD) as well as possibly larger molecular weight factors. This homogenate and fractions thereof is a potent activator. The homogenate will serve as screening agent (see below) for identifying inhibitors of cell activation. Identification of specific components thereof will permit preparation of antibodies for diagnostic purposes and also as targets for drug design and as screening agents to develop specific activation lowering agents.

A number of protease inhibitors were studied for their ability to inhibit pancreatic homogenate-induced neutrophil activation. Serine protease inhibitors were successful to varying degrees at preventing activation of neutrophils in vitro by pancreatic homogenate. Of these inhibitors, the serine protease inhibitor Futhan (nafamostat mesilate) proved the most efficacious. Experiments with neutrophils washed of unbound Futhan displayed similar inhibition to experiments where Futhan was added directly to homogenate, suggesting that the mechanism for Futhan inhibition of neutrophil activation is at the neutrophil membrane and is not necessarily directed that the homogenate itself. This conclusion is further strengthened by the observation that high concentrations of the principal pancreatic proteases trypsin and chymotrypsin (alone as well as in combination with their precursors trypsinogen and chymotrypsinogen) do not result in neutrophil activation in vitro. In addition, neutrophil activation was found in pancreatic homogenates filtered to remove factors greater than 3 kD, indicating that at least some of the neutrophil activating factors in pancreatic homogenate are of low-molecular weight, considerably smaller than any known proteases (approximately 20-300 kD). Activation was also retained in higher molecular weight pancreatic samples, but it is unclear at present whether this activity represents different factors, the low-molecular weight factor conjugated to a larger protein, or simply the low-molecular weight factor that remained after filtering.

As a control set of experiments, sub-activating concentrations of pancreatic homogenate were added to other organ homogenates liver, spleen, intestine, and heart that had previously shown little neutrophil activating ability. Surprisingly, incubation of these tissues with low concentrations of pancreatic homogenate resulted in their ability to strongly activate neutrophils. Further experiments demonstrated that this ability to activate neutrophils by previously inert organ homogenates could be duplicated by the addition of the pancreatic proteases chymotrypsin or trypsin. As previously mentioned, neither chymotrypsin nor trypsin intrinsically activate neutrophils in vitro, and heart, liver, spleen, and intestine homogenates have been shown to be non-stimulatory toward neutrophils. The addition of the proteases, however, resulted in the ability of these tissues to activate neutrophils in vitro. The mechanism behind this activation is currently unclear. It has been reported that platelet activating factor (PAF) can be activated by endothelium incubated with thrombin as well as chymotrypsin and cathepsin G), and can be inhibited by the addition of protease inhibitors. Therefore, this ability to stimulate neutrophils by homogenates incubated with serine proteases may be linked to their ability to synthesize PAF. Preliminary results suggest that there is little activity in the low-molecular weight fraction (<3 kD) corresponding to PAF, and PAF inhibitors have not been effective at reducing homogenate mediated neutrophil activation.

To relate the in vitro results obtained with pancreatic homogenate on neutrophil activation and its inhibition by serine proteases to the in vivo state, the SAO shock experiments were repeated using Futhan pretreatment. After optimal concentration and infusion parameters were determined, 60 minutes pretreatment of Futhan was found to mitigate the decrease in mean arterial pressure (MAP) seen after reperfusion (unclamping) in SAO shock. Mortality was reduced acutely and plasma levels of peroxide production were significantly lower than in saline-treated control rats. The mechanism of protection by Futhan appears to be due to a number of factors, including reducing neutrophil activation in vivo, stabilization of pancreatic lysosomal and acinar membranes, and an overall increase in the protective circulating anti-protease screen.

Injection of filtered pancreatic homogenate into animals closely simulated the MAP of the reperfusion phase in SAO shock, and resulted in increased circulating peroxide production as well immediate death, as seen in SAO shock. Pretreatment of animals with Futhan increased MAP in response to pancreatic homogenate injection and abolished the mortality seen in untreated animals. Injection of the low-molecular weight component of pancreatic homogenate also resulted in a sharp decrease in MAP. Blood pressure in these animals however, recovered after an approximately 10 minute hypotensive period and animals did not go into shock at the concentrations given (a maximum of 30% of the low-molecular weight component of one pancreas/animal). This decrease in blood pressure is most probably attributable to MDF, which is also a low-molecular weight substance and should thus be present in the low-molecular weight fraction along with neutrophil activating factors.

Intravital microscopy of the rat mesentery superfused with filtered pancreatic homogenate displayed a significant increase in neutrophil activation and vasoconstriction, conclusively demonstrating an in vivo role for pancreatic homogenate in the activation of not only neutrophils but other cell types. Cell death, endothelial and parenchymal, was not significantly increased in these experiments, suggesting that the pancreatic homogenate, while a source of neutrophil activating factors, is not directly cytotoxic to the tissues.

Different methodologies were used to isolate and identify the neutrophil activators found in the pancreas. Fast Pressure Liquid Chromatography (FPLC) was done on low-molecular weight pancreatic homogenate (<3 kD) in an effort to purify the neutrophil activating activity. Fractions were measured for their ability to activated neutrophils and ‘activating’ fractions were further purified via High Pressure Liquid Chromatography (HPLC). HPLC fractions were then analyzed for neutrophil stimulating activity and ‘activating’ fractions were then measured by MALDI mass spectroscopy. Known peptide sequences and low-molecular weight mediators were analyzed by computer and compared to the measured molecular weights. Based on these results, several known activators could be eliminated as low-molecular weight factors coming from the pancreas. Among those factors eliminated were PAF (also known as “authentic PAF” containing either a 16 or 18 carbon alkyl group), fMLP, bradykinin, angiotensin II, and all known cytokines. Several peptide sequences have molecular weights corresponding to those measured by mass spectroscopy, but none of these have been reported to possess any neutrophil stimulatory activity. Results from freezing pancreatic homogenate are inconsistent, but appear to decrease low-molecular weight activity. Lyophilization of low-molecular weight pancreatic homogenate has been unsuccessful as has dialysis, and preliminary attempts to extract activating factors in methanol or chloroform have likewise met with little success. The activity of the whole as well as low-molecular weight pancreatic homogenate is relatively stable at 4° C., and the whole molecular weight homogenate can be stored for at least one week. In contrast, factors produced by protease application to other organ homogenates are relatively unstable, with a half-life on the order of eight hours.

1. Tissue Homogenates Incubated with Serine Proteases Contain Factors that Activate PMNs In Vitro

Splanchnic arterial occlusion (SAO) shock results in upregulated levels of neutrophil (PMN) activation, as measured by pseudopod formation of donor PMNs exposed to shock plasma. Except for pancreatic homogenate, homogenates made of rat peritoneal organs do not significantly activate isolated naive PMNs. Pancreatic activation can be inhibited in vitro by addition of serine protease inhibitors.

Addition of exogenous proteases results in activation of other tissues. Rats randomly selected were weighed, anesthetized and catheterized. A laparotomy was made and the animals were exsanguinated. Organs were immediately removed into a 0.25 M sucrose solution and then homogenized in 1:9 (w/v) Krebs-Henseleit solution.

Organs harvested included spleen, proximal small intestine, pancreas, heart, and liver. Aliquots of each sample were mixed with serine proteases chymotrypsin and trypsin. The suspensions were incubated for 2.5 hours at 38° C. and PMN activation was measured. Results indicate a significant increase (p<0.01) in activation of PMNs by pancreatic homogenate as well as from tissue homogenates incubated with proteases (p<0.01). Activation from control organ homogenates other than the pancreas was not elevated. These results indicate that tissue homogenates incubated with serine proteases contain factors that activate PMNs in vitro. The pancreas may serve as an endogenous source for PMN activator(s).

2. Inhibition of the Activation In Vivo by FUTHAN

Plasma factors generated during splanchnic arterial occlusion (SAO) shock result in upregulation of leukocytes, as measured by nitroblue tetrazolium (NBT) or pseudopod activation. Homogenate from the pancreas, but less from other tissues will activate naive neutrophils by the same tests. This activation can be inhibited in vitro in part by serine protease inhibitors, such as FUTHAN (nafamstat mesilate ; a nonpeptidyl low molecular weight protease inhibitor 6-amidino-2-naphthy-p-guanidinobenzoate dimethanesulfonate; see, Fuji et al. (1981) Biochim. Biophys. Acta 661:342).

To demonstrate that activation is inhibited in vivo, rats were anesthetized and blood pressure monitored (MAP). FUTHAN was infused at the rate of 3.3mg/kg wt/hr. After one hour of pre-treatment, the superior mesenteric and celiac arteries were clamped for 90 minutes, at which time the clamps were removed. Animals were observed for survival for 60 minutes after reperfusion or until MAP fell below 30 mmHg. Plasma peroxide concentration was measured using an electrode technique.

Results indicate a significant difference in MAP after reperfusion between Futhan-treated and non-treated animals (p<0.005), as well as a significant increase in survival of Futhan-treated animals compared to controls (p<0.001). Peroxide levels in FUTHAN-treated SAO shock plasma were also significantly less than those in controls (p<0.05). The results indicate that SAO shock can be mitigated by pretreatment with a serine protease inhibitor and this protection may be derived in part from the ability of the protease inhibitor to limit the level of activators in the circulation during shock.

E. Cell Activation Assays

Rates of free radical production in whole blood can be measured using phenol red (Pick et al. (1980) J. Immunol. Methods 38:161-170) or other dye forming reagents (U.S. Pat. No. 5,518,891). Intracellular radical production may be measured with nitroblue tetrazolium (NBT) reduction or chemiluminescence (Cheung et al. (1984) Aust. J. Expt. Biol. Med. Sci. 62:403) assays. Radical production in whole blood or plasma may be measured electrochemically, and mRNA expression of specific genes can be quantitated, for example, using Northern blots or DNA micro arrays.

Expression of adhesion molecules such as CD11b, CD18, and of L-Selectin can be quantitated via flow cytometry, while cytokines and chemokines, such as interleukins and TNF-α can be quantitated with immunoassays.

Cell morphological changes may be quantified with direct microscopic examination, with or without fluorescent staining of F-Actin filaments present in pseudopods, or with fluorescence activated cell sorting techniques.

Blood plasma is known to carry cell activation factors in response to specific events. Plasma from I/R episodes including MI (Chang et al. (1992) Biorheology 29:549-561) and hemorrhagic shock (Elgebaly et al. (1992) J. of Thoracic and Cardiovascular Surgery 103(5):952-959; Paterson et al. (1993) Am. Vasc. Surg. 7(1):68-75; Barroso-Aranda et al. (1995) J. Cardiov Pharmacology 25(Suppl 2):S23-S29) activates neutrophils, as does plasma from smokers' blood (Pitzer et al. (1996) Biorheology 33(1):45-58). Patient blood samples can be applied to standard donor cells and the response of the donor cells used as a measure of the potency of the circulating activating factors in the patient blood.

F. Therapeutic Framework

Tests for activation would be empty without constructive responses to the information gleaned in the tests. Responses can take the form of adjustments to lifestyle and diet, such as increased exercise and lowered fat intake, postponement of scheduled surgery, anti-oxidant and activation-lowering drug therapy, or antagonists to circulating plasma factors. Examples of therapeutic decision trees are given in FIG. 2.

Nominally healthy patients with high activation could be counseled to adjust lifestyle and diet, or given an anti-oxidant (Stephens et al. (1996) The Lancet 347:781-786) or a relatively harmless activation-lowering therapy such as aspirin (Ridker et al. (1997) New England J. Medicine 336(14):973-979). High-risk surgery patients with high activation levels could postpone surgery or be given an activation-lowering therapy. An example of an existing protocol is the platelet aggregation blocker by Centocor (Reopro) given for high-risk angioplasty. Patients with unstable angina currently have choices ranging from no treatment to drug therapy to activation lowering or anti-adhesion (Husten, “Platelet receptor blockers effective for unstable angina,” Internal Med. World Report, May 15, 1997) drug therapy to angioplasty to bypass surgery. These choices could be guided by the degree of cell activation observed. Unstable angina has been shown, for example, to be associated with changes in neutrophil expression of CD11b and L-Selectin (Ott et al. (1996) Circulation 94(6):1239-1246).

In some cases high activation levels will be in response to infection. If the infection is subclinical, the activation test provides a clue to its presence. If the infection is apparent for other reasons, then treating it or waiting for it to subside becomes the first step in responding to high activation in non-critical care situations.

Finally, trauma and sepsis outcomes might be indicated by the presence of circulating plasma factors and by the extremity of the observed activation levels, so that choices of extreme interventions could be selected more rationally. Serine protease inhibitors such, as Futhan are effective in animal models in vivo against hemmorhagic shock, apparently block the effects of a factor originating in the pancreas. Thus, existing protease inhibitors should be useful for treatment of hemmorhagic shock of sepsis and should serve as drug targets.

The targets for treatment will be preferably either the factors, such as those released from the pancreas, that activate cells, or proteases that participate in the activation.

Treatment with Protease Inhibitors

Leakage of pancreatic proteases and other factors into the blood stream, or excessive activation in the pancreas without proper endogenous inhibitor control results in life treatening events. Injury to the pancreas generally is lethal and preventing protease action at the level of the white cell is known to be important for minimizing post-ischemic injury. Taken together, a drug that might be effective in preventing the generation of cell activation factors from tissues, to the extent that proteases play this role, should be therapeutic and have numerous clinical applications. This type of intervention has the potential to intervene early in the mediator/activation factor cascade and be particularly effective in minimizing post-injury phenomena.

Thus, methods of treatment of disorders and conditions related to inappropriate or chronic cell activation are provided. In particular, treatment by administration of effective amounts of broad protease inhibitors, particularly serine protease inhibitors are provided. In a preferred embodiment, the protease inhibitor is Futhan (nafamostat mesilate, which is 6-amidino-2-naphthyl p-guanidinobenzoate dimethanesulfonate) and treatment with a pharmaceutical composition containing an effective amount of Futhan is contemplated.

The protease inhibitors, such as Futhan or a similarly broad protease inhibitor, are used to treat patients in shock, suffering trauma or otherwise having compromised (i.e. individuals with activated circulating neutrophils) systems in order to minimize vessel/tissue injury. Administration is contemplated as soon as possible in the instance of a trauma or immediately prior to surgery or invasive clinical procedure in the case of compromised patients. The amounts administered (with reference to Futhan) are on the order of 0.001 to 1 mg/ml, preferably about 0.005-0.05 mg/ml, more preferably about 0.01 mg/ml, of blood volume by any suitable means, including intravenous, intramuscular, oral and parenteral administration. In an average adult, thus, about 50 mg of Futhan per dosage is administered. Since the compound is a low molecular weight drug and can be excreted relatively rapidly the frequency of treatment may be as often as every 6-8 hours during an acute episode or as little as one dose for a surgery patient. The precise amount of particular inhibitors administered can be determined empirically and will depend upon the particular disorder treated and outcome desired.

Care should be taken to monitor for bleeding and compromise of humoral host defense mechanisms. Futhanis relatively non-toxic and well tolerated in man.

G. Formulation and Administration of Active Compounds and Compositions

Compounds, such as protease inhibitors, including but not limited to serine protease inhibitors and Futhan, and compositions containing such proteases are provided herein. The compounds may be derivatized as the corresponding salts, esters, acids, bases, solvates, hydrates and prodrugs. The concentrations of the compounds in the formulations are effective for delivery of an amount, upon administration, that lowers cellular activation or inhibits cellular activation. Typically, the compositions are formulated for single dosage administration. To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and In vivo systems, such as the assays provided herein.

The concentration of active compound in the drug composition will depend on absorption, inactivation and excretion rates of the active compound, the physicochemical characteristics of the compound, the dosage schedule, and amount administered as well as other factors known to those of skill in the art.

Typically a therapeutically effective dosage The amounts administered are on the order of 0.001 to 1 mg/ml, preferably about 0.005-0.05 mg/ml, more preferably about 0.01 mg/ml, of blood volume Pharmaceutical dosage unit forms are prepared to provide from about 1 mg to about 1000 mg and preferably from about 10 to about 500 mg, more preferably about 25-75 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of the claimed compositions.

Preferred pharmaceutically acceptable derivatives include acids, salts, esters, hydrates, solvates and prodrug forms. The derivative is typically selected such that its pharmacokinetic properties are superior to the corresponding neutral compound.

Thus, effective concentrations or amounts of one or more of the compounds provided herein or pharmaceutically acceptable derivatives thereof are mixed with a suitable pharmaceutical carrier or vehicle for systemic, topical or local administration to form pharmaceutical compositions. Compounds are included in an amount effective for ameliorating or treating the disorder for which treatment is contemplated. The concentration of active compound in the composition will depend on absorption, inactivation, excretion rates of the active compound, the dosage schedule, amount administered, particular formulation as well as other factors known to those of skill in the art.

The compositions are intended to be administered by an suitable route, which includes orally, parenterally, rectally and topically and locally depending upon the disorder being treated. For oral administration, capsules and tablets are presently preferred. The compounds in liquid, semi-liquid or solid form and are formulated in a manner suitable for each route of administration. Preferred modes of administration include parenteral and oral modes of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as Tween®, or dissolution in aqueous sodium bicarbonate. Derivatives of the compounds, such as prodrugs of the compounds may also be used in formulating effective pharmaceutical compositions. For ophthalmic indications, the compositions are formulated in an opthalmically acceptable carrier. For the ophthalmic uses herein, local administration, either by topical administration or by injection is preferred.

Time release formulations are also desirable. Typically, the compositions are formulated for single dosage administration, so that a single dose administers an effective amount.

Upon mixing or addition of the compound with the vehicle, the resulting mixture may be a solution, suspension, emulsion or or other composition. The form of the resulting mixture depends upon a number of factors, including the intended mode of administration and the solubility of the compound in the selected carrier or vehicle. If necessary, pharmaceutically acceptable salts or other derivatives of the compounds may be prepared.

The compound is included in the pharmaceutically acceptable carrier in an amount sufficient to exert a therapeutically useful effect in the absence of undesirable side effects on the patient treated. It is understood that number and degree of side effects depends upon the condition for which the compounds are administered. For example, certain toxic and undesirable side effects are tolerated when treating life-threatening illnesses that would not be tolerated when treating disorders of lesser consequence. The concentration of compound in the composition will depend on absorption, inactivation and excretion rates thereof, the dosage schedule, and amount administered as well as other factors known to those of skill in the art.

The compounds can also be mixed with other active materials, that do not impair the desired action, or with materials that supplement the desired action, such as cardiovascular drugs, antibiotics, anticoagulants and other such agents known to those of skill in the art for treating cardivascular disorders, shock, infection, trauma and other disorders in which cellular activatin is implicated in a causal or contributory role. Thus, the protease inhibitor, such as Futhan, may also be advantageously administered for therapeutic or prophylactic purposes together with another pharmacological agent known in the art to be of value in treating one or more of the diseases or medical conditions referred to hereinabove, such as beta-adrenergic blocker (for example atenolol), a calcium channel blocker (for example nifedipine), an angiotensin converting enzyme (ACE) inhibitor (for example lisinopril), a diuretic (for example furosemide or hydrochlorothiazide), an endothelin converting enzyme (ECE) inhibitor (for example phosphoramidon), a neutral endopeptidase (NEP) inhibitor, an HMGCoA reductase inhibitor, a nitric oxide donor, an anti-oxidant, a vasodilator, a dopamine agonist, a neuroprotective agent, a steroid, a beta-agonist, an anti-coagulant, or a thrombolytic agent. It is to be understood that such combination therapy constitutes a further aspect of the compositions and methods of treatment provided herein.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these formulations are known to those skilled in the art.

The active compounds or pharmaceutically acceptable derivatives may be prepared with carriers that protect the compound against rapid elimination from the body, such as time release formulations or coatings.

Finally, the compounds, such as the serine protease inhibitors, such as Futhan, may be packaged as articles of manufacture containing packaging material, a compound or suitable derivative thereof provided herein, which is effective for antagonizing the lowering cell activation, within the packaging material, and a label that indicates that the compound or a suitable derivative thereof is lowering cell activation. The label can optionally include the disorders in which cell activation is implication or treatment protocols in which cell activation therapy is warranted.

H. Drug Screening Assays and Cell Activation Assays

The pancreatic homogenate or subfractions thereo, particularly the fractions that contain active components with molecular weights less than about 3 kD, may be used to screen for compounds that inhibit cellular activation. The homogenate is contacted with a suitable cells such as an endothelial cell line or neutrophils, or selected tissue, and the cells are assayed to assess the level of activation. Test compounds that reduce the level of activation can be identified by contacting the cells with the homogenate simulaneously, after or before contacting the cells with a test compound. Those that reduce the level of activation relative to the homogenate in the absence of the compound are selected for further investigation. In other embodiments, the effects of the test compounds are compared with known inhibitors, such as Futhan and other serine protease inhibitors, of the activity of the homogenate or fractions thereof. Compounds that inhibit substantially well or more than the known inhibitors are selected for further evaluation.

Other Assays

Donor cells or cell cultures responding to patient blood plasma samples can be used show cell activation behavior, clastogenic (mutagenic) activity, apoptotic potential, effects on intercellular junctions such as relevant to the blood-brain barrier, and general gene transcriptional effects.

Once circulating plasma factors are isolated and identified, antibodies to these factors will provide specific assays.

Another method in which patient plasma assayed for its ablility to activate neutrophil as an indication of the presence of cell activation is provided herein (see, Example 6). It can be used in the classical fashion; that is, fresh patient blood is centrifuged and the plasma measured for superoxide formation. In another embodiment, control plasma from healthy individuals can be used as a vehicle to test activation of different substances, even other patient plasma. This latter method provides neutrophils in autogolous plasma and obviates the need for large amounts of patient plamsa. As little as 100 μl of plasma (and possible less using the new smaller volume configuration) can be measured for its ability to activate otherwise quiescent neutrophils. This method can give accurate results in as little as 1 hour (10 minutes centrifugation, 10 minutes setup and 40 minutes of measurement). Becauase the number of neutrophils in spun plasma is much less than that of isolated neutrophils in autologous plasma, the relative levels of chemiluminescence are likewise attenuated. In normal (control) plasma, all values thus far (>100 experiments with more than 5 different donors) have had a maximum repsonse of between 1500 and 6000 counts/sec ina time frame of 20-50 minutes. The normal range is approximately 3000+/−500 counts/sec in approximately 40 minutes. This can be modified by donor illness, antibiotics, and more interestingly, ingestion of fatty diet.

These assays alone or in combination can be used to identify other factors and/or to assess levels of cell activation, which will be related to disease outcomes and can be used to support useful therapeutic decisions. Other assays for measuring cell activation levels in patient samples, include any cell activation known to those of skill in the art, and particularly those exemplified herein.

J. Generation of Additional Therapeutic Targets

As exemplified below, although homogenates from tissues, other than pancrease did not yield cell activation factors, treatment of tissues with the pancreatic factors provided herein and also proteases, particularly serine proteases, resulted in activation. Thus, other targets for drug screening may be generated by treating selected tissue with the pancreatic composition or active fractions thereof or with a protease inhibitor, and then using purification procedures as described herein for the pancreatic homogenate, isolating active fractions, and ultimately the active factors from other tissues.

The following examples are included for illustrative purposes only and are not intended to limit the scope of the invention.

### EXAMPLE 1

Introduction

This Example presents a short introduction of research done on the subject of neutrophil activators.

As noted above, the body produces factors that either lead or contribute to pathologic conditions in the organism. In disease conditions, there occurs an upregulation of host defense responses by the cells (circulating as well as tissue (e.g., endothelium, mast cells)) in the body. Prominent among the upregulated cells are the leukocyte neutrophils, which in their capacity as second line of defense (after the physical skin and mucous membrane boundaries), possess a formidable capacity to injure the body itself. There exists a fine line between the ability to readily destroy and phagocytose invading pathogens, necessary for survival of the organism and inappropriate activation of these cells. In the past, before the advent of antibiotics, death by sepsis was a very common occurrence. The more active the neutrophil response was toward invasive bacteria the more chance the host stood of surviving the attack. As antibiotics have greatly reduced the fatality rate in response to infection, people live longer and are more prone to diseases such as atherosclerosis and hypertension that are rarely expressed in younger individuals. There is thus a tradeoff between neutrophil activation necessary to prevent outside infection and inflammatory regulation sufficient to avoid ultimately damaging the host. Neutrophil activation, while a necessity for defense against pathogens, is also responsible for tissue destruction and organ damage seen as a consequence of its activation. This “auto-immune response” is implicated in many pathologies, including cardiovascular diseases (myocardial infarction, stroke, atherosclerosis, hypertension), arthritis, sepsis, trauma, and shock. The causes of the upregulation of cells in the body seen in response to different stimuli are not well understood. Certainly there are many mediators implicated in the upregulation of host defense and countless factors, cytokines and bacterial products have been observed to activate neutrophils in vitro and in vivo, but common or generalized mediators have not be clearly identified.

Neutrophil activation serves not only as host response against foreign antigens, but is also involved in reactions that are frequently deleterious to the host. Research has focused on factors that activate neutrophils in vitro and in vivo (see, Wientjes et al. (1995) Semin Cell Biol 6:357-651; Ley (1996) Cardiovasc Res 32:733-42; Downey et al. (1995) Semin Cell Biol 6:345-356). Most studies that address this issue assume a catastrophic event or a reoccurring chronic illness as the trigger mechanism that upregulates these cells. It has been observed that neutrophil activation occurs in the absence of recognizable pathologies and this resultant “preactivation” can have deleterious consequences to the host in the event of a traumatic event or other stressor. Neutrophil preactivation appears to be seasonal in nature, with activation levels peaking in the winter months and reaching a minimum in the summer months. This may be related to observed seasonal increases in other potentially deleterious circulating factors including lipids and fibrinogen. Furthermore, variables, such as time of day, exercise and especially, diet, can influence baseline levels of neutrophil activation and affect the circulating levels of (neutrophil) inflammatory products such as superoxide. For example, it was observed that plasma from otherwise healthy blood donors given meals rich in saturated fats the night previous produces upregulated levels of neutrophil activation compared to plasma from the same subjects after a low-fat meal.

The mechanisms that affect the neutrophil quiescent state appear to be due to a combination of factors, as the neutrophil is very sensitive to changes in its ambient environment and is easily activated.

1.1 Preactivation and Priming

With the knowledge that activated neutrophils are implicated in the pathogenesis of disease, the link between neutrophil activation and acute trauma, using hemorrhagic shock or acute endotoxemia as the insult has been examined herein. Not only are neutrophils activated systemically in shock, but there exist plasma factors that will induce activation when incubated with naive donor neutrophils, thus demonstrating conclusively the presence of circulating neutrophil activators (see, Barroso-Aranda et al. (1992) Circ Shock 36:185-190; Barroso-Aranda et al. (1989) Am J Physiol:H846-852; and Shen et al. (1990) Circulatory Shock 31:343-344).

Neutrophils in vivo circulate as a heterogeneous population that includes nonactivated, ‘primed’, and activated cells. Primed cells are those cells that have been subjected to a sub-threshold stimulus and are now hyper-responsive to any additional stimulus. There are some investigators who maintain that priming is necessary before neutrophils can be activated in vivo (i.e. the necessity of having two stimulatory events) and there is some evidence to support this. On the other hand, any stimulus with sufficient magnitude will also stimulate the neutrophil directly. Thus, the relative importance of priming in vivo is not yet clear, nor is it known to what extent circulating neutrophil activators are ‘primers’ for additional stimuli. Most probably, circulating neutrophil activators shift the population distribution towards greater numbers of activated and primed cells, at the expense of the non-activated population. This can be illustrated by the experiments (Barroso-Aranda et al. (1989) Am J Physiol:H846-852), that measured neutrophil superoxide production (an index of neutrophil activation) in animals subjected to hemorrhagic shock. Superoxide levels were increased before and after shock in nonsurviving animals compared to surviving animals, indicating that high numbers of primed and activated cells previous to insult (shock) lead to even greater levels of activated cells after insult (as the total activated cells are now the combined population of primed plus activated neutrophils).

What is of even greater interest, however, is the finding that initial levels of neutrophil activation by circulating plasma factors correlate inversely with survival of those animals in shock compared to otherwise identical animals. This suggests that in otherwise matched subjects, there exist differences in individual neutrophil activation levels that may lead to higher levels of mortality in those subjects with greater neutrophil “pre”-activation, and that this preactivation is due in part to circulating humoral factors.

It is proposed herein, that this activation is a focal point for therapeutic intervention and also in treatment protocol assessment.

1.2 Objective

Under well controlled experimental conditions, there exist significant differences in tissue damage and survival rate after exposure to shock between otherwise matched subjects. Animals with high initial levels of the preactivators have lower survivability to not only hemorrhagic shock, but also septic shock. These preactivators appear to stimulate (upregulate) cells in the cardiovascular system. The biochemical nature of the factors is not well defined, but they appear to be substances carried in the plasma. Plasma with high levels of preactivation will activate naive neutrophils, as determined by superoxide production and actin polymerization tests, while plasma with low levels of the activators show less such reaction.

It is proposed herein that these activators cause, in addition to increased mortality in shock, increased oxygen free radical production during reperfusion and resultant higher levels of lipid peroxidation and cell death. These activators may be present endogenously in tissue and be released in response to sub-clinical perturbations to tissues, most notably diet, exercise, stress, and foreign pathogens. Circulating activators shift the neutrophil population distribution towards the activated state, resulting in increased tissue damage in under chronic conditions and mortality in the acute state. Thus, if such levels ascertained, the treatment modalities and outcome of treatment can be predicated by assessing these levels.

1.3 Importance of Endogenous Neutrophil Activating Factors

The importance of neutrophil activating factors produced during shock and found endogenously in the tissue is described herein. The understanding of the functions of these factors in vitro and in vivo rleads to a greater awareness of their actions during shock and in healthy individuals. With the understanding of the mechanisms of their actions, strategies can be devised to interfere with inappropriate neutrophil activation by these factors, whether in the form of acute interventions or day-to-day adjustments in health care maintenance.

### EXAMPLE 2

Neutrophil Cell Activation: Definition and Quantification

Neutrophils are implicated in the pathology of a number of disease processes, acute and chronic. In order for these cells to exert their deleterious effects on the host, they must first become activated. “Activation” of neutrophils represents a change in the quiescent or “normal” state to one which includes upregulation of oxidative metabolism, increased intracellular calcium concentrations, morphological shape changes induced by cytoplasmic protein polymerization, and finally, degranulation of cytoplasmic granules. In vivo these processes may not be coupled, and different stimuli can induce different degrees of upregulation of these parameters.

Thus, the term “activation” must be defined in terms of specific parameters. For these studies, superoxide production (as defined by the nitroblue tetrazolium test and lucigenin-enhanced chemiluminescence), and actin polymerization (defined by the pseudopod formation test) have been selected as indices of neutrophil activation. These two responses are uncoupled. In resting-state neutrophils there is little correlation between “activation” as measured by the two types of measurements. As the stimulation to neutrophils is increased, this correlation increases demonstrably. Thus, the use of two different parameters in defining “activation” gives a wide assessment of neutrophil upregulation.

2.1 Methods for Assessing Neutrophil Cell Activation

When exposed to soluble stimuli neutrophils become “activated.” Neutrophil activation can be expressed by a number of parameters that are upregulated under inflammatory conditions, including actin polymerization, superoxide formation, cell degranulation and protease release (Ferramte et al. (1992) Immunol Ser 57:499-521; Ley (1996) Cardiovasc Res 32:733-42, Chatham et al. (1994) J. Leukoc Biol 56:654-660), and upregulation of adhesion molecules (Ley (1995) Bioeng Sci News 18:43-47; Murohara et al. (1995) Cardiovasc Res 30:965-974; and Jaboson et al. (1993) J. Immunol. 151:5639-5652). Although these indices of activation are not necessarily coupled, when subjected to sufficient stimuli, neutrophils will tend to display all of these attributes.

For these studies actin polymerization, superoxide and hydrogen peroxide formation were used to define the activation state of a neutrophil population. This response to stimuli can take different forms, including the upregulation of the oxidative burst mechanism (NADPH oxidase), actin polymerization (from globular or g-actin to filamentous or f-actin), expression of adhesion molecules and degranulation of the lysosomal granules. Which mechanism is upregulated depends in part, on the stimulus to which the neutrophil is subjected. For example, leukotriene B₄ (LTB₄) and complement fragment C5a are potent chemo-attractants but poor stimulators of the oxidative response, as is ATP.

Other cytokines, such as interleukin-1 (Il-1), neutrophil-activating protein-1/interleukin-8 (NAP-1/Il-8), tumor necrosis factor-α (TNF-α), granulocyte/macrophage colony-stimulating factor (GM-CSF), and γinterferon (γ-IFN) are also poor direct stimulators of NADPH oxidase. These factors, like the bacterial product lipopolysaccharide (LPS), however, are potent “priming” agents that potentiate the oxidative response to another stimulus.

Environmental factors such as osmolarity changes and excessive shear stress can prime neutrophils as well, although conversely it has been found that physiologic levels of fluid shear may be necessary to keep neutrophils from being upregulated in the circulation. The common activating peptide formyl-methionyl-leucyl-phenylalanine (fMLP) and platelet activating factor (PAF) did not activate the respiratory burst of isolated neutrophils in the absence of plasma (see Example 4), suggesting the necessity of sufficient intracellular (PMN) ATP in order to activate the respiratory burst in response to these stimuli.

In most inflammatory conditions there is a concomitant upregulation of most if not all “activation” mechanisms of neutrophils, either due to multiple stimuli or due to an overabundance of one stimulus or both (Badwey et al. (1991) Adv Exp Med Biol 314:19-33). There was little correlation between tests in non-activated neutrophils. There appears to be minimal correlation between actin polymerization (pseudopod production) and the oxidative burst as measured by the NBT test in quiescent neutrophils.

The tests were made using addition of rat plasma from animals subjected to SAO shock to isolated human neutrophils for the pseudopod formation test and addition of the same plasma to whole rat (donor) blood for the NBT test. Repeated tests using whole human blood for NBT test show similar correlations.

For the studies exemplified herein, neutrophil activation is defined by the oxidative burst and by actin polymerization. The oxidative burst component was measured using lucigenin-enhanced chemiluminescence and the nitroblue tetrazolium (NBT) test, both of which are sensitive to the generation of superoxide and can be blocked by superoxide dismutase (SOD). The test for actin polymerization relies on detection of pseudopod formation, which is accompanied by a cell deformation from a spherical state into a polarized shape.

The combination of these two assays measure two different components of neutrophil activation, and generally correlate when assessing cells that have been subjected to a stimulus. Both types of tests have a narrow sensitivity, especially the NBT and pseudopod formation tests, which are limited to no more than two orders of magnitude, since measurements fall between 0 and 100% and all cell counts are of the order of 100 cells in each test. The use of two different kinds of assays however, lends a reasonable certainty to classifications of “activated” and “non-activated” cell populations.

2.2 The Oxidative Burst

The neutrophil oxidative burst is due to the upregulation of the membrane-bound nicotenamide adenine dinucleotide phosphate (NADPH) oxidase system, which converts oxygen to superoxide (a free radical) via the reaction:

NADPH+2O₂→NADP⁺+2O₂−+H⁺ (2)

Free radicals, molecules with an unpaired electron, are quite reactive and are known to cause tissue damage due to breakdown of cell membranes, denaturing of proteins and destruction of nucleic acids (see also Example 3.1.b). Superoxide and its dismutated product, hydrogen peroxide (H₂O₂), are oxygen free radical constituents formed by activated neutrophils. Superoxide is not intrinsically reactive and although it is thought to be produced predominantly extracellularly, is does not easily cross cell membranes except perhaps through ion channels. Hydrogen peroxide on the other hand, is more stable and able to pass freely through cell membranes, but it is minimally toxic at physiological concentrations (<1 mM) and may not account for the extent of oxidative cell injury incurred by activated neutrophils (apart from degranulation). It is the interaction between these two species that is thought to produce the cytotoxicity of free radical-induced oxidation, catalyzed by iron and other bivalent metals to form the potent hydroxyl radical, which will react with virtually all biological substances. O₂− is produced in large amounts; 2×10⁶ neutrophils stimulated with 10⁻⁸ M fMLP have been reported to produce 10 nmoles O₂⁻ in 1 minute in a volume of 1-2 μl. This is equivalent to the production of approximately 5-10 mM O₂−/minute. Superoxide spontaneously dismutates (albeit at a slow rate) to hydrogen peroxide, or more rapidly in the presence of superoxide dismutase (SOD).

Superoxide and hydrogen peroxide then react in the Haber-Weiss reaction, creating the highly reactive hydroxyl radical:

O₂−+Fe³⁺→O₂+Fe²⁺

Fe²⁺+H₂O₂→Fe³⁺+OH⁻+OH⁻

The Haber-Weiss reaction, however, occurs slowly in vivo (Halliwell et al. (1990) Methods Enyzmol. 186:1-85) catalyzed by a transition-state metal ion. The metal-catalyzed Haber-Weiss or Fenton reaction, is believed to be the mechanism by which superoxide and hydrogen peroxide contribute to cell death.

Support for this theory comes from observations that SOD and catalase, an enzyme that degrades hydrogen peroxide, decrease neutrophil-mediated oxidative injury in many systems, by inhibiting O₂− and hydrogen peroxide formation, respectively. This inhibition is generally effective whether only SOD is used, only catalase is used, or a combination of the two is used. Depending on the organ and injury model studied, differences arise with respect to the relative effectiveness of each inhibition. A final toxic component of the neutrophil respiratory burst is the formation of hypochlorous acid (HOCl), formed by the reaction of H₂O₂ with a halogen such as chlorine in the presence of the neutrophil azurophilic granule enzyme myeloperoxidase.

2.3 Methods: Measurement of the Oxidative Burst

The quantification of the neutrophil oxidative burst is made by the reaction of superoxide with another substrate, either lucigenin or nitroblue tetrazolium, to produce a product that can be easily measured. NADPH oxidase is activated via a number of mechanisms, receptor mediated and non-receptor mediated. Examples of stimuli that are receptor mediated include fMLP, C5a and TNF-α. Non-receptor mediated stimuli include calcium ionophores, protein kinase-C (PKC) activators such as phorbol myristate acetate (PMA), G-protein agonists and surface active stimuli such as detergents and arachidonic acid.

In the experiments, described herein, of NADPH oxidase-mediated cell activation, the known receptor mediated activators PAF and fMLP as well as the pancreat activating factor compositions provided herein, were used.

2.3.a Chemiluminescence Assay

The chemiluminescence assay using lucigenin to enhance superoxide-produced chemiluminescence is discussed in detail in Example 6.

2.3.b Nitroblue Tetrazolium Test

In the NBT-test, a count is made of the percent of circulating neutrophils from a naive donor that are able to spontaneously reduce the pale yellow NBT to blue-black formazan crystals in the presence of donor plasma (Shen et al. (1990) Circulatory Shock 31:343-344). NBT reduction by neutrophils has been shown to be associated with enhanced superoxide production (Shen et al. (1990) Circulatory Shock 31:343-344). Pretreatment with superoxide dismutase (from bovine erythrocytes, Sigma Chemical Company, St. Louis, Mo.) blocks this reaction (Barroso-Aranda et al. (1989) Am J Physiol:H846-852).

To perform this test aliquots of 0.4 ml of the test plasma or activator are collected per sample. Fresh arterial blood from a donor animal (0.1 ml) or heparinized human venous blood from a healthy volunteer is mixed with 0.4 ml of the test plasma or activator and immediately transferred into a clean siliconized 1 dram glass vial (Sigma Diagnostics, St. Louis, Mo.) and mixed with an equal amount of 0.1% NBT-solution. This mixing procedure avoids centrifugation of donor neutrophils, a step that induces spontaneous activation. The ratio of 0.1 ml whole blood at approximately 40% hematocrit and 0.4 ml plasma assures that the donor neutrophils are exposed to a concentration equivalent to at least 80% of that in plasma from the tested (e.g. shocked) animals. Plasma exchanges are not associated wit visible abnormal red cell reactions or cell aggregation. The glass vial is then incubated at 37° C. in air for 10 minutes and subsequently allowed to stand at room temperature for an additional 10 minutes. At the end of this period, the blood-NBT mixture is gently stirred. Coverslip smears are made and stained with Wright's stain. A total of 100 neutrophils are routinely counted under 1000× oil objective magnification. Neutrophils that show a stippled cytoplasm with deposits of formazan or a dense clump of formazan are counted as NBT-positive cells Slides are measured in duplicate or triplicate and results averaged. In a light micrograph of a typical non-stimulated rat neutrophil in non-activated rat donor plasma, no NBT crystals are seen. The cells are stained with Wright's stain. In a light micrograph of a rat neutrophil stimulated by addition of activated rat donor plasma, NBT crystals are visible in the cytoplasm.

A modification to the standard NBT test protocol was made submitting the crystal violet nuclear stain for Wright's stain. This stain has the advantage of only staining the nucleus of white blood cells, making the identification of neutrophils a relatively straightforward process. Care must be taken in the use of this stain since it tends to dissolve the blood smears if not carefully applied. Use of this stain as well as NBT (formazan) on standard “wet amounts” (isolated PMNs for pseudopod determination) reveal a much greater percentage of NBT (+) cells than normally detected using Wright's stain (data not shown), due to accurate visualization of NBT crystals not only in pseudopods but in the intact spherical cells as well. The percentage of cells that are NBT (+) using the latter method approaches 100% even for quiescent cells, confirming that even non-activated neutrophils continuously produce at least basal levels of superoxide. NBT counts using the crystal violet stain are noted where applicable, and care must be exercised in direct comparisons of these counts with previous results using Wright's stain.

2.3.c Peroxide Production Measurements

In some plasma samples the concentration of peroxide, presumably attributable to hydrogen peroxide, was determined. Although there exist (circulating) producers of peroxide in isolated plasma (see, e.g., Example 3), the majority of this radical product is assumed to come from superoxide-producing neutrophils. Using an electrode technique, it has been possible to measure levels of (hydrogen) peroxide produced in activated plasma after blockade of catalase with sodium azide. Briefly, this method uses a platinum anode biased against a silver/silver chloride reference cathode. A sample of heparinized blood (1.5 ml) is drawn and separated into two 0.75 ml aliquots and immediately put on ice (0-4° C.). These are then incubated for 10 minutes at 37° C. and centrifuged for 10 minutes at 500 G at room temperature. Measurements are then made in the supernatant plasma fraction with sodium azide (20 μl of a 2 M stock solution) added to the first sample's plasma layer and catalase (20 μl containing 0.25 mg of the enzyme, (Aspergillus niger in 3.2 M (NH₄)₂SO₄, pH:6.0, Sigma Chemicals, St. Louis, Mo.)) to the second. After gentle stirring, the electrode is placed in the plasma of the appropriate aliquot and its output is recorded for 10 minutes, at which time a steady-state signal has been reached. Sodium azide (Sigma Chemical Company, St. Louis, Mo.) inactivates plasma enzymes including catalase, which degrade hydrogen peroxide. Catalase degrades hydrogen peroxide into oxygen and water. The current obtained from the sample with catalase is thus subtracted from the sample with sodium azide, and the difference between the currents is ascribed to hydrogen peroxide production.

Exogenous hydrogen peroxide in blood samples is measured with an electrochemical sensor. Measurements are made in the supernatant plasma with sodium azide and catalase. The current measured in the catalase sample is subtracted from the current in the azide sample, to yield a current resulting from the hydrogen peroxide in the sample. The sensor has a platinum anode biased at 0.6 V with respect to the silver/silver chloride cathode. Hydrogen peroxide reacts at the surface of the anode producing an electrical current that is proportional to the peroxide in solution.

This system is calibrated by placing the electrode in 2 ml buffered saline solution and two plasma samples (containing 20 mM sodium azide). Known concentrations of hydrogen peroxide are added to the solutions and the electrode current is monitored. A linear response for the current is between 0 and 10 μM. The equation determining actual peroxide concentrations is given by:

Peroxide Concentration (μM)=(Vazide−Vcatalase)*10.43+0.045

as determined by a least-squares fit from the calibration curve.

2.3.d Pseudopod Formation

The pseudopod formation measurement is used determine the percentage of neutrophils (PMNs) that display pseudopods due to actin polymerization. Difficulties, noted below, may arise when interpreting pseudopod formation that may occur due to non-specific cell membrane activators such as detergents. Care should be taken to avoid such activators.

To isolate human neutrophils, plasma is separated from red blood cells by sedimentation and neutrophils are isolated by a Percoll-gradient technique. Because neutrophils are sensitive to changes in their physical environment, particular care must be taken to not agitate the cells. Care includes avoiding the common dextran-70 sedimentation technique and the changing of buffer osmolarity in order to lyse red blood cells. While these techniques may not overtly activate the neutrophil layer, this kind of treatment will actively prime them.

For the pseudopod assay, venous blood is collected in heparinized tubes from healthy human volunteers and put on ice. It is important that heparin and not EDTA (ethylamine diamine tetraacetic acid) be used as an anticoagulant, as the calcium-chelating properties of EDTA can affect neutrophil activation. Rat neutrophils have a density comparable to rat red blood cells and therefore neutrophil isolation of rat PMNs is considerably more time consuming and difficult. After sedimentation in an appropriate sized sterile syringe while on ice (60 ml), the plasma containing white blood cells and a minimum of red blood cells is layered onto a 3.5 ml Histopaque (Sigma Chemical Company, St. Louis, Mo.) fluid layer in 12 ml polypropylene centrifuge tubes (17×100 mm, Falcon, Shrewsbury, Mass.) and centrifuged for 20 minutes at 600 G. Sedimented red blood cells and neutrophils are gently resuspended in 2 ml D-PBS or alternatively, Krebs-Henseleit buffer (118 mM sodium chloride, 4.7 mM potassium chloride, 2.5 mM calcium chloride, 1.2 mM magnesium sulfate, 1.2 mM potassium, titrated to pH 7.4 with Tris Trizma-HCl (Sigma Chemical Company). Other chemicals for the Krebs-Henseleit buffer are from Fisher Scientific, Fair Lawn, N.J. The resuspended cells are then gently layered onto 2.5 ml of a 55% isotonic Percoll (Sigmal Chemical Company) solution and 2.5 ml of a 74% isotonic Percoll solution in deionized water. The suspension is centrifuged for 15 minutes at 600 G and the middle granulocyte layer is removed and resuspended in PBS to achieve a concentration of 10⁶ neutrophils/ml. 100 μl aliquots of suspended neutrophils are added to 100 μl of test plasma or activating agent. This mixture is mixed and then incubated for 10 minutes at 27° C. After incubation 100 μl of 3% glutaraldehyde (Fisher Scientific) is added to stop the reaction. 100 μl of crystal violet in phosphate (pH: 7.4) buffer is then added to stain leukocyte nuclei on wet mount preparations. Freely suspended neutrophils with pseudopodia are identified by their segmented nuclei and the presence of cytoplasmic granules. One hundred neutrophils are counted. Cells with pseudopod projection greater than about 1 um are considered positive. Repeated measurements indicated that such counts are reproducible within 2%.

For some experiments using pseudopod formation tests, the following modified procedure was used. Results from the two methods differed slightly and are most probably attributable to inter-observer variations (Table 2.1, below).

2.3.e Neutrophil Isolation on Ficoll/Hypaque (Pfeifer Method)

A single medium (A) or discontinous gradient of two media (A and B) may be used. For medium A, 44 g of Ficoll 400 (Pharmacia no. 17-0400-01, Piscataway, N.J.) are dissolved in 440 ml of water (which yields about 460 ml of solution). The density of this solution is measured with a pyknometer (around 1.0303 g/ml at 20° C.) and then sterilfiltered. 24 ml of Hypaque-76 (Sanofi/Winthrop no. NDC 0024-0776-04, containing 66% diatrizoate meglumine and 10% diatrizoate sodium, 1.432 g/ml) are added to every 100 ml of this solution. 15 ml of the mixture are removed and the density is measured again with the pyknometer. The value obtained should be 1.1061-1.1063 g/ml. It can be adjusted by adding more Ficoll solution or more Hypaque-76 to decrease or increase density, respectively. This medium is slightly hypertonic. Medium B is the commercially available Ficoll-Paque (Pharmacia no. 17-0840-03, Piscataway, N.J.) for lymphocyte isolation and has a lower density than medium A. It contains 5.7 g of Ficoll 400 and 9.0 g of sodium diatrizoate per 100 ml of solution.

TABLE 2.1
Comparison of Pseudopod Methods With Different

| Observer 1 | Observer 2 | |
|---|---|---|
| (Method 1) % | (Method 2) % | Difference % |
| 4 | 2 | 2 |
| 1 | 4 | 3 |
| 2 | 2 | 0 |
| 10 | 5 | 5 |
| 22 | 7 | 15* |
| 8 | 8 | 0 |
| 24 | 27 | 3 |
| 10 | 6 | 4 |
| 3 | 4 | 1 |
| 10 | 4 | 6 |
| 7 | 9 | 2 |

Comparison of pseudopod formation assay results from the two methods described in the text as obtained by different observers. These assays were done on HPLC separation columns. The large difference in the single sample (*) resulted from different interpretations of detergent-induced (TFA and acetonitrile) polarization.

Whole blood is drawn from a (male)* donor into a syringe containing EDTA pH 7.3 (10 mM final concentration). In 50 ml conical tubes, 30 ml of this blood are layered with 2-3 ml of medium B and 12 ml of medium A. The tubes are then spun at 750 G for 25 minutes at 20-24° C. without braking the centrifuge spinning head at the end of the 25 minutes to avoid disturbance of the layers. The neutrophil band (between mononuclear and red cells) is removed and washed in Earle's balanced salt solution (EBSS) without calcium or magnesium, containing 9 mM morpholinopropanesulfonic acid (MOPS) pH 7.35. A second wash is performed with a 1:1 mixture of EBSS without Ca²⁺ and Mg²⁺ and regular EBSS (both with MOPS). The cells are finally taken up in regular EBSS with MOPS, counted and checked fro pseudopod formation. This method yields about 6-30×10⁶ neutrophils/10 ml of whole blood. Contaminating cells are predominantly of red blood cells with some mononuclear cells (1-5% of isolated leukocytes). The whole isolation procedure requires approximately 90 minutes.

The neutrophils are counted, diluted to 1.1×10⁶/ml and left at room temperature for five minutes. 100 μl of activator (pancreatic homogenate, fMLP, etc.) are added to 900 μl of cell suspension in concentrations designed to achieve the final working concentration. A timer is started and after two minutes 100 μl of this suspension are added to 125 μl of ice cold glutaraldehyde (2.5% in NaCl 0.9%) in the wells of a microtiter plate. The cells are left to sediment in the cold and are counted (100 per well) to determine the percentage of polarized neutrophils. Cells are examined under 400× and those deviating from the typical spherical shape are scored as being polarized. Results are expressed as percent of polarized cells per total cells counted (100 cells counted per sample, except as indicated).

Most cell separation procedures were performed using the first described separation technique. Variations from this procedure are noted when applicable.

2.4 Discussion

In practicing the diagnostic and treatment assessment methods provided herein, it will be necessary to determine as accurately as possible the activation state of neutrophils to a variety of stimuli, in vitro and in vivo. The in vitro techniques discussed here represent several of the available methods currently used to assess the neutrophil activation. Other methods may also be used.

Since they are biological assays, and the NBT and pseudopod tests in particular rely in part on observer objectivity and accuracy, difficulties may arise when relying solely on the results from one kind of test. Thus, often, particularly if results are not clear, more than one test will be used. Also, as discussed above, the different forms of neutrophil activation in response to stimulation are not necessarily coupled or causal in nature. Therefore for the experiments described herein, within practical limits of time and materials, as many of the four different tests were applied in an effort to determine more precisely the magnitude and nature of neutrophil activation.

As noted aboved, neutrophils are extremely sensitive to their environment and are easily activated. Also, activation as assessed by the NBT and pseudopod formation tests is necessarily binary in nature, i.e., a cell is either activated or not. Under normal environmental conditions this is not an issue, but difficulties may arise when cells are subjected to a non-physiologic environment, as is the case when activation is determined with high performance liquid chromatography (HPLC) filtered samples or organic/inorganic phase separations.

Therefore, care has been taken in making sure that the medium in which activation measurements are made is as physiologic as possible. This typically involves dose-dependent tests to determine the amount of solvent allowable in a given measurement paradigm. Unless germane to the discussion, these calibration tests are not reported here. Activation of control samples with calibrated amounts of solvent is, however reported and labeled as such.

### EXAMPLE 3

### Hemorrhagic Shock and the Presence of Activating Factors

Summary

Hemorrhagic hypotension is a well-studied model of acute trauma involving the concerted actions of activated neutrophils, oxygen free radicals, inflammatory cytokines and other circulating mediators, the uncontrolled production of which result in lipid peroxidation and cell death. In this global ischemia-reperfusion paradigm, upregulation of activators in shock plasma measured as as increases in plasma peroxide levels, lipid peroxidation and cell death, not only during the reperfusion component, but also to some extent during the hypotensive period, have been observed. Correlations among these groups suggest not only synergy between their actions, but also call into question common assumptions about the temporal progression of hemorrhagic shock. In particular, the involvement of activating factors, during the shock process, and in “preactivation” of plasma before shock may prove to be a major determinant in the course and progression of acute trauma.

3.1 Introduction

3.1.a Hemorrhagic Shock

Global ischemia and subsequent reperfusion lead to complications that are accompanied by cell and organ damage. Tissue damage after hemorrhagic shock depends on the degree of pressure reduction, the choice of anesthesia (if applicable), as well as duration of ischemia and the nature of the organs affected. Some organs, notably skeletal muscle, may survive periods of up to four hours of ischemia without adverse effects. Others, such as those in the splanchnic region and brain, are more sensitive and do not tolerate low-flow states for an extended length of time. Organs such as the heart can tolerate limited ischemia for short durations.

There is a time window in which reperfusion is desirable and clinically possibly relevant, since not all cells are killed and salvage may be possible. Interventions against ischemia-reperfusion, including hemorrhagic shock must be made during the ‘treatment window’ or before when tissue is still salvageable. After this time, injury is irreversible regardless of intervention (Sussman et al. (1990) Methods Enzymol 186:711-783). Shorter durations of ischemia followed by reperfusion result in less impairment of tissue function, while longer periods of ischemia may lead to cell death and tissue necrosis, whether or not there is reperfusion. Total occlusion of a vessel, as opposed to low flow states, leads to predominantly anoxic cell death rather than free radical interactions when reperfusion is not obtained (McCord (1986) Adv. Free Rad Bio & Med 2:325-345).

In hemorrhagic shock as well as ischemic states in general, the decrease in blood flow results in reduced oxygen transport to tissue as well as impaired waste product removal. These factors lead to impaired function and eventually death of the tissue. Paradoxically, the replacement of shed blood in the case of hemorrhagic shock, or the re-establishment blood flow to previously ischemic tissue leads to the phenomenon known as “reperfusion injury.” This injury, appears to be due to the reoxygenation of previously ischemic tissue and production of oxygen free radicals and other toxic substances. Free radicals, molecules with an unpaired electron, are highly reactive and are known to cause tissue damage due to breakdown of cell membranes, denaturing of proteins and destruction of nucleic acids. Although oxygen free radicals have been implicated in reperfusion injury, which free radicals are involved and their site of production has not been resolved. The prevailing hypothesis holds that hypoxia caused by low blood flow and subsequent oxygen exchange in ischemic tissue leads to activation and upregulation of otherwise benign enzymes and production of free radical species in larger amounts.

3.1.b Oxygen Free Radical Production

Chief among the producers of these free radicals is believed to be the endothelial membrane-bound enzyme xanthine oxidase (XO), which by limited proteolysis is either reversibly or irreversibly converted from xanthine dehydrogenase (XD), which uses NADH, to xanthine oxidase, which uses O₂ to drive the reaction. Hypoxia causes XD to be converted to XO, while increased ATP catabolism increases both of the substrates for XO/XD, xanthine and hypoxanthine. Upon reperfusion, oxygen is once again readily available and xanthine and hypoxanthine are degraded by XO to uric acid. In the process, the free radical superoxide (O₂−) and hydrogen peroxide (H₂O₂), are released in an approximate ratio of 30:70. Reactions of xanthine (and hypoxanthine) with xanthine oxidase produce superoxide and hydrogen peroxide:

Xanthine+3O₂+2H₂O₂→2O₂⁻+2H⁺+H₂O₂.

Under quiescent conditions xanthine oxidase exists as a xanthine dehydrogenase and reacts with NAD+ to form NADH and uric acid.

Circulating XO has also been implicated as a participant in global ischemia/reperfusion injury. Other possible sources of oxygen free radicals include mitochondrial cytochromes, which are probably inactivated by ischemia, and NADH oxidase. Also involved with the reperfusion oxygen free radical reactions are neutrophils, which secrete O₂− via membrane-bound NADPH, as well as release a host of membrane-degrading proteases and other substances (see Example 2). Regardless of the mechanism, superoxide and hydrogen peroxide are appear to be the major oxygen free radical constituents formed by reperfusion injury.

Questions remain with this “free radical theory of toxicity” however, due in part to differences in tissues and species studied, as well as experimental protocol. The rat, for example, appears to have high XO levels in tissues such as the intestine, leading to pronounced injury in intestinal ischemia/reperfusion. In contrast, XO is reported to be produced in human cardiac tissue only in insignificant quantities. In spite of this evidence, the XO inhibitor allopurinol is effective in myocardial ischemia/reperfusion, apparently due to other actions of the drug or possible inhibition of circulating XO. Questions also remain as to why SOD reduces tissue injury when in fact it increases the relative proportion of hydrogen peroxide, a more toxic species than superoxide. This is perhaps explained by adequate catalase levels in some tissues (and red blood cells) that can inactivate these increased levels of hydrogen peroxide. Since superoxide is necessary for the Fenton reaction, the application of SOD may mitigate organ injury by eliminating one of the necessary components of this reaction.

Lipid peroxidation is an “oxidative deterioration of polyunsaturated lipids” (Holley et al. (1993) Br Med Bull 49:494-505). This deterioration typically involves the abstraction of electrons from a carbon-carbon double bond in an unsaturated lipid and is mportant process in free radical mediated reactions and subsequent cell death. First characterized in the 1940s, lipid peroxidation is a ubiquitous oxidative process seen not only in pathological disease conditions but in everyday life, eg., the “rancidity” that affects foods, polymers and plastics. In living tissues, the cell membranes undergo lipid peroxidation. Cell membrane structure in tissue differs in each organ as to its lipid makeup but is typically composed of a lipid-to-protein ratio of the order of 1:1, while the mitochondrial membranes are somewhat higher in protein concentration, at approximately 80%. Most lipids are phospholipids containing a glycerol base and a polar tail region. The non-polar head is a fatty acid composed of long carbon groups, usually from 14-20 carbons long, attached by an ester. Double bonds are in the cis formation, resulting in long straight chains. The more unsaturated a fatty acid is, the more susceptible it is to oxidative attack. Arachidonic acid is a common 20 carbon fatty acid with double bonds at C5, C8, C11, and C14 and is a common inflammatory mediator released by such cytokines as the prostaglandins. Because of its four double bonds it is a primary target of oxidative attack.

The first step in lipid peroxidation, assuming a normally peroxide-free medium, is known as the first chain initiation step, where a hydrogen ion is abstracted from a methylene (—CH2—) group by a strong oxidizing agent such as the hydroxyl radical. This leaves a free electron on the carbon (—C H—), which is now a free radical as well. From this, especially in polyunsaturated lipids such as arachidonic acid, conjugated dienes result, propagating the free electron species down the fatty acid chain until coming to rest at a stable endpoint, typically near the end of the chain. Here, lipid radical interaction with O₂ results in a peroxy radical (CHO₂) which then can abstract another hydrogen ion, resulting in an self-perpetuating autocatalytic reaction. The lipid with the hydrogenated peroxy (peroxyl) radical is now a lipid hydroperoxide, which can decay further, reacting with itself to become a cyclic peroxide and then degrading to a cyclic endoperoxide. A final (stable) end-product after reaction of endoperoxides with oxygen and subsequent hydrolysis is malondialdehyde (MDA), a three carbon molecule with oxygen double-bonded at both ends. These end products are not uniformly degraded. For example, arachidonic acid degradation due to oxidative attack results in at least six lipid hydroperoxides as well as cyclic peroxides and other products.

The requirements for lipid peroxidation are not completely known, but iron-catalyzed (Fenton) reactions are thought to play a major role. Hydroxyl radicals can easily initiate site-specific hydrogen ion abstraction as well as encourage continued peroxide autoxidation. Hydroxyl radical formation does not seem to be required to initiate peroxidation, since OH— scavengers do not inhibit this process. Iron is important in later aspects of lipid oxidation as well. Lipid peroxides (R—OOH) readily react with iron(II)-bound complexes, resulting in an oxidized iron(III)-bound complex, plus OH⁻ and an alkoxy (alkoxyl) radical (R—O—). In addition, the oxidized ferric iron-complex can react with lipid peroxides as well, albeit at a much slower rate, forming peroxy radicals and a ferrous iron-complex, thus essentially recycling the iron to be used again. The alkoxy and peroxy-radicals can abstract hydrogen ions and stimulate lipid peroxidation. These iron reactions with lipid peroxidation compete favorably with the Haber-Weiss reaction, with a K₂ of 76 m⁻¹s⁻¹ for hydrogen peroxide, compared with a K₂ (2nd order rate constant) of 1.5×10³ m⁻¹s⁻¹ for lipid hydroperoxides with ferrous iron complexes.

The number of iron containing proteins that promote lipid peroxidation is much greater than that available for Fenton hydroxyl formation. Among the molecules that bind iron that stimulate lipid peroxidation include ATP, carbohydrates, DNA, and membrane lipids. This intracellular iron is also available for the Haber-Weiss reaction. In contrast, tightly bound iron-containing molecules, which is where the overwhelming majority or cellular and extracellular iron is stored, is not available for Fenton reactions (unless the iron is released) but can contribute to lipid peroxidation reactions. Among these proteins are ferritin, hemosiderin, lactoferrin, transferrin and the heme proteins. The availability, especially of heme proteins would seem to point to the red blood cell membrane as a prime target for lipid peroxidation. Hemoglobin in red blood cells is sequestered near high concentrations of catalase and glutathione reductase, apparently to limit this sort of process.

3.1.d Methods for Measuring Lipid Peroxidation

Because of the difficulty in measuring lipid peroxidation directly in vivo, several methods of determining relative lipid peroxidation have been developed. Among the most popular of these is the TBARS, or thiobarbituric acid reactive substances assay (Darley-Usmar et al. (1994) The Biochemist 18:15-18; Portoles et al. (1993) Biochim Biophys Acta 1158:287-92; McKenna et al. (1991) Anal Biochem 196:443-450; Kosugi et al. (1994) Biol Pharm Bull 17:1645-1650; Wallin et al. (1993) Anal Biochem 208:10-15; Severn et al. (1993) Eur J immunol 23:1711-1714; Augustin et al. (1991) Life Sci 49:961-968; Vasankari et al. (1995) Clin Chim Acta 234:63-69; Sandhu et al. (1992) Free Radic Res Commun 16:111-122; Yagi et al. (1984) Methods Enzymol 105:328-331). The TBARS assay uses thiobarbituric acid under acid conditions, which when heated, forms a chromogen whose color intensity at 532 nm is directly proportional to the amount of reactive substance formed. Originally developed to measure the amount of malondialdehyde (MDA), an ultimate end-product of lipid peroxidation, the TBARS assay also reacts with other substances to form the chromogen. Among these postulated adducts include deoxyribose, protein linkages and amino acid compounds. Unwanted reactions can be eliminated or minimized with the use of phosphotungstic acid-sulfuric acid to precipitate proteins and lipids and the use of acetic acid instead of trichloroacetic acid (TCA) to avoid reactions with sialic acid. The exact conditions are important in conducting the TBARS assay, as the TBARS test is susceptible to oxidation, and depends on the antioxidant status and iron content of serum as well as amount of lipids, making storage of samples at −70 C a critical factor. Despite these difficulties, the TBARS assay is a straightforward method for determining relative lipid peroxidation, and correlates well (slightly overestimating) with HPLC (high performance liquid chromatography) methods. Because the TBARS test is calibrated with NMA (1,1,3,3,-tetramethoxypropane is hydrolyzed for actual measurement as MDA itself is unstable), results from the assay are typically expressed in amount of MDA produced, or simply in units of absorbance.

3.2 Methods

3.2.a Experimental Protocols—Hemorrhagic Shock

Male Wistar rats (250-350 gm, Charles River Laboratories, Inc., Wilmington, Mass.) were housed in a controlled environment and maintained on a standard pellet diet for at least three days before initiation of experimental procedures. Animals were cannulated via the femoral arteries and vein (PE-50 polyurethane tubing, Clay Adams, Parsippany, N.J.) under general anesthesia using pentobarbital (50 mg/kg i.m., Abbott Laboratories, North Chicago, Ill.) and placed on a custom-built Lucite stage. No heparin was injected other than that required to ensure open catheter lines (10U/ml Plasma-Lyte, Upjohn Comp., Kalamazoo, Mich.). One femoral artery was connected up to monitor mean arterial pressure (MAP) and pulse pressure (Beckman Instruments). For rat mesentery experiments a central incision was made over the abdomen and the mesentery was carefully placed on the intravital microscope stage with a minimum of handling.

In the hemorrhagic shock experiments, it was found that the thin mesentery preparation would not exhibit reperfusion injury exposed to the open atmosphere, possibly due to ambient oxygen diffusion. Therefore a specially fitted plastic sheet (3 mm thickness) was fitted over the animal preparation and attached to the stage using Velcro straps, with small openings for head and tail, as well as catheter lines. Two other openings served for placement of the microscope objective and insertion of gas. Nitrogen gas was infused, ensuring a hypoxic environment on the mesentery preparation. The mesenteric microcirculation was observed through intravital fluorescence microscopy (Technical Instruments; San Francisco, Calif.) during superfusion (1.0 ml/min) with Krebs-Henseleit bicarbonate-buffered solution saturated with 95% N₂-5% CO₂ gas mixture (118 mM sodium chloride, 4.7 mM potassium chloride, 2.5 Mm calcium chloride, 1.2 mM magnesium sulfate, 1.2 mM potassium, 25 mM sodium bicarbonate. Chemicals were from Fisher Scientific, Fair Lawn, N.J.)

After 15 minutes for stabilization of MAP and pulse pressure, propidium iodide (PI) (1 μM) (Sigma Chemical Co., St. Louis, Mo.) was added to the superfusate and background autofluorescence was recorded in selected tissue areas. A first reading was then taken of bright-field and fluorescent images of selected venules and arterioles (20 μm-100 μm). 4-5 observation fields were selected at random and readings were recorded every 20 minutes with the use of a digital color coupled charge device (CCD) camera (Optronics Engineering; Goleta, Calif.) and a 40× water immersion objective (Zeiss; Thornwood, N.Y.) connected to a color video monitor (Panasonic CT 1383 VY, Japan) and cassette recorder (Panasonic, AG-1270, Japan). Images were recorded for later analysis. Fluorescence light excitation exposure time was minimized to avoid photobleaching.

After a 20 minute stabilization period (5 min after addition of PI to superfusate), hypotension was induced by a stepwise reduction in the blood volume taken from a femoral artery catheter over a period of 20 minutes until the MAP reached 40 mmHg. Thereafter, small aliquots of blood were either removed or heparinized Plasma-Lyte was injected to keep MAP within the specified level of hypotension over a period of 100 minutes. The blood volume that was removed during the bleeding and hypotensive period was at least 3% of body mass. Following a 100 minute hy-potensive period, the blood that had been removed was rewarmed in a 37° C. water bath and returned by slow intravenous infusion over a period of 20 minutes. Blood withdrawn for serum analysis was replaced in equal or slightly greater volume with Plasma-Lyte (approx. 2 ml).

3.2.b Measurements

MAP and heart rate were recorded throughout the shock protocol. Arterial blood samples were collected in heparinized vials at intervals before and after hypotension and reperfusion. 0.5 ml samples were collected at t=0 minutes and 230 minutes (2 hours after reperfusion) for NBT tests. In addition, in selected animals 1.75 ml samples were collected at t=0, 60, 100, 115 min (15 min after reperfusion), and 230 min for measurement of lipid peroxidation and plasma peroxide. Samples were immediately centrifuged for 30 minutes at 1000 G and plasma separated from cells. Plasma was then stored at −70° C. The plasma of rats before and after hemorrhagic shock was tested on naive donor leukocytes in whole blood obtained from rats that were not exposed to hypotension. Nitroblue tetrazolium reduction by the leukocytes due to superoxide production was then tested. For such a test, 0.1 ml of donor whole blood was mixed with 0.4 ml plasma from the rats in hemorrhagic shock. The donor animals, anaesthetized with pentobarbital (50 mg/kg i.m.) were cannulated via the femoral artery (PE-50 polyurethane tubing). The mixture of reconstituted blood was incubated for 10 minutes at 37° C. and then subjected to the NBT test. Neutrophil actin polymerization, using the pseudopod formation assay was also measured. The tests are described in Example 2.

3.2.c Measurement of Cell Death in the Mesentery

Video tapes were replayed for analysis of cell death, as determined by PI fluorescence. Venules were restricted to 20-80 μm in diameter for analysis. The number of PI-positive cells was calculated at initial time points in 4-5 arbitrarily defined regions of the mesentery, taken every 20 minutes. The entire field-of-view was used for this purpose, approximately, 300 μm×300 μm. The number of dead (PI positive) endothelial cells in the representative vessel was also noted. The number of dead cells were compared at different time periods throughout the experiment.

3.2.d Plasma Assay for Lipid Peroxidation—TBARS Assay

Plasma lipid peroxidation was measured on arterial samples collected at regular time intervals during hypotension and reperfusion. As described above, 0.25 ml aliquots of blood were collected and immediately centrifuged at 1000 G for 30 minutes. Plasma and red blood cells were separated and immediately stored at −70° C. until analysis. For the TBARS assay, a modified method based on Yagi ((1984) Method Enzymol 104:328-331) was used. For this method, 100 μl of plasma was mixed with 2 ml of N/12 H₂SO₄ and gently shaken. Then 0.25 ml of 10% aqueous phosphotungstic acid was added and mixed. This mixture was allowed to sit for 5 minutes and was centrifuged at 3000 rpm for 10 minutes. The supernatant was discarded and the sediment was again mixed with 1 ml of N/12 H₂SO₄ and 0.15 ml of phosphotungstic acid. This was mixed once more and centrifuged at 3000 rpm for 10 minutes. The supernatant was then discarded and the sediment was mixed with 1 ml purified H₂O and 1 ml of TBA reagent, composed of equal volumes of glacial acetic acid and 0.67% thiobarbituric acid aqueous solution. The resulting mixture was heated for 60 minutes at 95° C. in a water bath. After cooling, 2 ml of n-butanol was added to the mixture and shaken vigorously. After centrifugation at 3000 rpm for 10 minutes, the supernatant was decanted into clear plastic vials for spectrophotometric measurement (Perkin-Elmer Lambda 3B spectrophotometer). Measurements were taken at 532 nm and at 600 nm against distilled water and the resulting absorbances subtracted from each other to give a relative absorbance value. This absorbance value was then linearly interpolated with a least squares fit calibration curve using known values of MDA to give an absorbance in terms of MDA concentration (Curve waw calibrated with malonaldehyde (MDA), an endproduct of lipid peroxidation. Absorbance was measured at 532 nm).

3.2.e Plasma Peroxide Assay

The plasma peroxide concentrations were measured at times t=0, 60, 90, 120, and 230 minutes throughout the shock protocol. Plasma samples were collected as described above and peroxide concentration was measured as detailed in Example 2, section 2.3.c with the essential difference that the peroxide concentrations measured in the shock protocol were derived solely from plasma and not from the plasma layer of centrifuged blood, which remains in contact with the sedimented cells. Thus, measured plasma peroxide in these samples that have been centrifuged for 30 minutes at 1000 G (normal centrifugation for this procedure is 500 G for 10 minutes) and then frozen at −70° C. is not cell-derived (there are no cells present) nor was the production disrupted by freezing of the plasma.

All chemicals were purchased from Sigma Chemicals, St. Louis except where indicated. Results are expressed as Mean±SD for all samples. A two-tailed unpaired Student's t-test was used for all comparisons. Differences with P<0.05 were considered significant.

3.3 Results

Representative time courses of mean arterial pressure (MAP) for survivors (n=3) and non-survivors (n=5) subjected to hemorrhagic shock were performed. After 10 minutes equilibration blood was slowly withdrawn in a step-wise fashion until MAP achieved 40 mmHg. During the hypotensive period blood was returned or withdrawn as needed to obtain a continuous MAP of 40 mmHg±5 mmHg. At reperfusion blood pressure of survivors returned to a sustainable pressure, whereas in non-survivors blood pressure rose transiently and eventually fell irreversibly despite repeated efforts to restore blood volume and pressure. The results showed that the mean arterial pressure of non-survivors tends not to recover after reinfusion of shed blood, even when adequate volume replacement is provided. Also, this group is more likely to necessitate preliminary infusion of blood or Plasma-Lyte during the hypotensive period. By contrast, the MAP of survivors tends to return to near pre-shock levels after reperfusion.

Application of rat shock plasma (n=3) to donor rat blood resulted in a significant increase in NBT(+) neutrophil counts (P<0.001) compared to plasma taken from the same rats before the shock protocol (n=8). Neutrophil activation as assayed by the nitroblue tetrazolium test for superoxide production by neutrophils before (t=0) (n=8) and after (t=230 minutes) (n=3) 100 minutes of hemorrhagic shock and 120 minutes of reperfusion was compared. There was a significant increase in neutrophil activation measured by NBT in the plasma from shocked rats.

A set of representative images taken using the intravital microscope on the rat mesentery preparation were preprared and showed a large increase in PI positive cells after 20 minutes of reperfusion as well as venule vasoconstriction. DCFH fluorescence (n=3) however, was not significantly different from control preparations undergoing mock hemorrhage procedures (n=3). Reperfusion after hemorrhagic shock resulted in a significant increase in mesentery cell death as measured using propidium iodide in all cells viewed in the preparation (P<0.05 compared to initial values after reperfusion until termination of the experiment, n=5 fields, n=2 animals as well as in endothelial cells alone. The results revealed that there is little increase in cell death as measured by propidium iodide until the start of the reperfusion period, when there is a sharp increase in the amount of cell death. This increase remains sustained until the end of the experiment.

Endothelial cell death lags behind generalized (interstitial) cell death. Cell death in the endothelium does not appear to coincide with that of the preparation in general, and there is a delay of almost an hour after parenchymal cell death before there is significant endothelial death. This finding was surprising in light of the fact that the endothelium is not only exposed early on to circulating toxins but is also a major producer of free radicals in shock. A comparison between the two plots in each graph showed evidence in favor of the contribution of increased neutrophil “preactivation” levels to cell injury.

Other expermiments measured time course of peroxide formation in the plasma peroxide concentration in rats subjected to hemorrhagic shock as measured ex vivo using a peroxide electrode technique. A small increase in plasma peroxide during hypotensive period followed by large jump after reperfusion of shed blood was noted. Levels are low until late in the hypotensive period when there is a rise in peroxide concentration, followed by a sharp increase in plasma peroxide directly after reperfusion. The overall levels recorded are lower than normal levels due to modification of testing procedure using frozen plasma in the absence of cells rather than the plasma layer of centrifuged blood. The time course of lipid peroxidation during the global ischemia and subsequent reperfusion period was performed by a time course of TBARS production, a measure of lipid peroxidation, as measured by absorbance at 532 nm during hemorrhagic shock. Levels were increasing before reperfusion of shed blood, but increase abruptly after reperfusion and subsequently decline. As with the cell death measurements and plasma peroxide measurements, there is a slow increase in plasma TBARS concentration throughout the hypotensive period. The concentration is substantially increased upon the reperfusion phase.

3.4 Discussion

Increased levels of neutrophil “preactivation” are correlated with increased mortality in hemorrhagic shock (see, Barroso-Aranda et al. (1992) Circ Shock 36:185-190; Barroso-Aranda et al. (1989) Am J Physiol:H846-852; and Shen et al. (1990) Circulatory Shock 31:343-344). Although “preactivation” has been observed in these cases there is presently little understanding of the mechanisms underlying the increased mortality observed in animals with raised levels of a “preactivator”. The observation that activating factors occur in circulating plasma indicates that “preactivation” is a systemic phenomenon. Thus, it is possible that “preactivation” may lead to several forms of organ dysfunction and over longer periods of time may be responsible for upregulation of certain autoimmune and host defense responses.

The “free radical theory of toxicity” is supported by compelling evidence implicating it as the lethal mechanism in global ischemia/reperfusion. If raised levels of a “preactivator” result in increased mortality in global ischemia/reperfusion, it is likely that one of the mechanisms for this increase in mortality is the upregulation of oxygen free radical-producing systems and the subsequent overproduction of toxic free radical species. It is known that membrane degradation either by free radical oxidation or other factors can form biologically active mediators which, among other functions, activate neutrophils. Chief among these neutrophil-activating lipids is platelet activating factor (PAF) or PAF-like substances, which can be formed via oxidative damage to cell membranes (see, also, Example 9.)

Hemorrhagic shock (Wiggers' model) is a well-studied but still incompletely understood model of acute trauma that appears to involve the upregulation of neutrophils and other cells, free radical interactions, lipid peroxidation, cell dysfunction and ultimately, organ death. Although there are undoubtedly synergistic actions between these events, the relative importance and temporal course of activation of these variables is unclear. It has been shown (Suematsu et al. (1994) Lab Invest 70:684-695) that cell death as visualized by propidium iodide in skeletal muscle during hemorrhagic hypotension increasesd due to endothelial derived free radical production before significant leukocyte accumulation. This, however does not negate the possible primary role of neutrophils in other organs, notably the splanchnic region and lungs, which may in turn produce circulating mediators that ultimately affect the more hardy skeletal muscle.

The results shown here support the conclusion that, as neutrophil activation is increased after hemorrhagic shock, plasma peroxide production is increased, and subsequently lipid peroxidation and cell viability may be affected. It is likely that these events are coupled. A linear correlation between the degree of neutrophil activation present before the shock process as well as after reperfusion and the relative amount of plasma lipid peroxidation detected was observed. This correlation suggests that lipid peroxidation as detected here may not simply be a passive byproduct of cellular free radical attack and subsequent cell death but a major contributor to neutrophil activation and possible “preactivation”. The correlation between degree of “preactivation” as assessed by NBT(+) superoxide production and lipid peroxidation as measured by TBARS was observed. Results indicated a significant degree of correlation between degree of NBT(+) activation before (r2=0.992) and after shock (r2=0.838), suggesting a possible relationship between the two events.

The findings that increased levels of neutrophil activators appear in the plasma (see above) before cell death in a vulnerable tissue such as the mesentery and in the form of “preactivation” point towards circulating neutrophil activators as significant constituents in the shock process.

### EXAMPLE 4

### Splanchnic Arterial Occlusion Shock and Plasma Activation in the Rate

Summary

The link between splanchnic arterial occlusion (SAO) shock and the presence of neutrophil activating factors in plasma from SAO shocked animals is described in this Example. Rats were randomly divided into, shock and shock sham groups. A laparotomy was made an in shock animals, the superior mesenteric and celiac arteries were occluded. After a period of 90 minutes, the clamps were removed and the splanchnic region was reperfused. SAO shock was verified by a precipitous fall in systemic blood pressure upon reperfusion. Aliquots of blood were taken before occlusion and after reperfusion, and measured for NBT activity and neutrophil pseudopod formation, indices of leukocyte activation.

Results indicated a significant increase (p<0.001) in activation of leukocytes by plasma from SAO shocked animals in both sets of assays. Plasma from sham shock rats displayed no increase in activation. These results show that plasma activation occurs in SAO shock, and demonstrates that a humoral activation factor is derived from the splanchnic region, in particular the gut and pancreas, that may be co-localized with myocardial depressant factor, also found in the pancreas.

4.1 Introduction

As shown in Example 3, there exist powerful “activating factors” of cardiovascular cells in the plasma of animals subjected to hemorrhagic and endotoxic shock whose presence not only increases markedly in these shock states but also correlates with diminished survival in models of hemorrhagic shock. It was not known, however, whether splanchnic arterial occlusion (SAO) shock would induce upregulation of leukocyte activation. This form of ischemia/reperfusion injury is important in that it isolates the splanchnic region as possible precursor site for the formation of activating factors. The finding of neutrophil activation in such a sock model may lead to insights into the origin of neutrophil activators. This study was designed to determine whether splanchnic arterial occlusion shock would induce activation of cardiovascular cells.

SAO shock is a form of shock which involves the splanchnic region by clamping one or more of the major supply arteries to this region. The main artery supplying the splanchnic region is the superior mesenteric artery, which arises directly from the aorta and feeds the pancreas, duodenum and mesentery of the small intestine. Occlusion of this vessel results in uniform mortality in dogs within 12-48 hours. One of the hallmarks of this model is that occlusion of the superior mesenteric artery is often fatal even before the intestine has lost its viability. Furthermore, the release of the occlusion leads to death more certainly and rapidly than if the tissue had maintained ischemic. The latter observation points to the susceptibility of the splanchnic region to “reperfusion injury”, either from free radical interactions or other circulating toxic metabolites. Because of collateral flow from other vessels, especially the celiac artery and inferior mesenteric artery, much of the intestine remains viable when only the superior mesenteric artery is occluded, permiting much of the splanchnic region to remain viable for several hours.

The model of splanchnic arterial occlusion shock used in these experiments involves clamping the superior mesenteric artery as well as the celiac artery. The celiac artery supplies collateral flow to the superior splanchnic region (such as the pancreas) and ischemia to both arteries results in a much quicker and more uniformly lethal outcome than occlusion of the superior mesenteric artery alone. The third major supply vessel to the splanchnic region, the inferior mesenteric artery can also be clamped, but this results in large intestine and bowel necrosis which was unwanted in this study because of possible bacterial translocation. Clamping the superior mesenteric and celiac arteries insures almost complete ischemia to the pancreas while leaving the large intestines relatively well perfused. This model of SAO shock has been well studied and is quite reproducible.

SAO shock, a well-established model with a more circumscribed region of tissue exposed to ischemia/reperfusion was chosen to determine whether or not circulating neutrophil activators would be reproduced in the splanchnic region. Ischemia/reperfusion in the splanchnic region results in the release of myocardial depressant factor (MDF). Upon reperfusion, MDF circulates and depresses cardiac contractility, resulting in compromised cardiac function, reduction of blood pressure, and exacerbation of shock. This factor has been recognized to be released in part from the pancreas, is exquisitely sensitive to low-flow states, becomes ischemic readily under conditions of low-flow, in part due to shunting of blood flow to the more ‘critical’ organs (heart, brain). Thus, either a global hypotension or a direct ischemic episode in this organ results in the release of MDF. It was hypothesized that the circulating neutrophil activating factors found during hemorrhagic shock might also be produced in SAO shock, and could possibly be co-localized with or even identical to MDF.

4.2 Methods

Male Wistar rats (250-350 gm, Charles, River Laboratories, Inc. Wilmington, Mass.) were housed in a controlled environment and maintained on a standard pellet diet for at least three days before initiation of experimental procedures. Rats were randomly divided in SAO shock (n=10) and SAO shock sham groups (n=11). Animals were cannulated via the femoral arteries and vein (PE-50 polyurethane tubing, Clay Adams, Parcippany, N.J.) under general anesthesia using pentobarbital (50 mh/kg i.m., Abbot Laboratories, North Chicago, Ill.). No heparin was injected other than that needed to ensure open catheter lines (10 U/ml plasma-Lyte, Upjohn Comp., Kalamazoo, Mich.). A femoral artery was cannulated to monitor mean arterial pressure (MAP) and pulse pressure (Beckman Instruments, Ill.). A central incision was then made over the abdomen. The superior mesenteric and celiac arteries were isolated. SAO shock was induced by total clamping of these arteries. In sham (control) animals the arteries were isolated but not clamped. The abdominal cavity was closed and covered with a layer of gauze soaked in warm saline. At the end of the shock period (90 minutes) the central incision was reopened and in the shock animal group the clamps were removed. Arterial blood samples were collected in heparinized vials before and after SAO shock in the SAO shock and the sham groups. Samples were immediately centrifuged for 30 minutes at 1000 G and the plasma was separated from the blood cells. Plasma was stored at −70° C. for further analysis.

The plasma of rats before and after SAO shock as well as in the sham controls was tested against naive donor leukocytes in whole blood obtained from rats without exposure to shock. Nitroblue tetrazolium reduction by the leukocytes due to superoxide production was measured. For such a test, 0.1 ml of donor whole blood was mixed with 0.4 ml plasma from the rats in SAO shock as well as SAO shock shams. The donor animals were anaesthetized with pentobarbital (50 mg/kg i.m.) and were cannulated via the femoral artery. Naive control blood was incubated with experimental plasma for 10 minutes at 37° C. and hen subjected to NBT test as described in Example 2. The NBT reduction is due to superoxide formation and can be clocked with superoxide dismutase (SOD).

Determination of human neutrophil pseudopod formation due to application of shock plasma was also made as described in Example 2. Pseudopod formation is due to actin polymerization, another index of neutrophil activation.

For determination of myocardial depressant factor in the shock plasma, pooled samples of SAO shock and SAO shock sham plasma were filtered at 1300 G through a 3000 kD filter (Centricon, Amicon Corp., Beverly, Mass.) and the filtrate from ten such pools (average number of animals per pool=6) was sent elsewhere on dry ice in randomly numbered vials, along with a control of Krebs-Henseleit solution to be analyzed for myocardial depressant factor (MDF). Samples were assayed for the depressant factor activity on electrically driven isolated papillary muscles taken from the right ventricle of cat hearts. Developed tension of the isolated papillary muscles were recorded on an oscillographic recorder. MDF formation was recorded as the absolute value percentage change in contractility f the cat papillary muscle at standard conditions of 37° C. and a frequency of stimulation of 1 Hz., as reported in (Lefler (1970) Clrc Res 26:59-69).

4.3 Results

Occlusion of the superior myenteric and celiac arteries for ninety minutes is a reproducible model of intestinal shock that results in uniform mortality to rats (n=16) within two hours after removal of clamps. The shock was characterized by an immediate increase in blood pressure of approximately 6-12 mmHg when the arteries were clamped. Mean arterial blood pressure (MAP) remained elevated throughout the experiment and the animal appeared to maintain normal cardiac and lung activity until the claps were removed, at which time there was a precipitous fall in blood pressure to approximately 40 mmHg (P<0.05 compared to shock sham group (n=4)). This pressure then decreased gradually until the animals expired, typically between about 10 minutes and about two hours. Mean arterial pressures of SAO shock group and sham shock in rats exposed to procedure was measured. Anincrease in blood pressure when the arteries of shocked animals are occluded and a precipitous fall when clamps are removed were observed. Sham shock animals display no significant changes in blood pressure. Mortality was 100% at two hours.

SAO shock results in the formation of neutrophil activating factors in plasma as determined by the NBT test (P<0.001 compared to shock sham group and shock animals before shock protocol) and pseudopod formation (P<0.001 as compared to both shock sham group and shock animals before the shock protocol). Percent of neutrophils from donor blood displaying pseudopods induced by plasma from SAO shock and Sham shock before (Initial) and after (Final) shock were measured.

There was no significant enhancement in neutrophil activation measured in control animals by either assay during the course of ischemia and reperfusion. Myocardial depressant activity was increased in all but one SAO shock samples as compared to control plasma samples (P=0.065) by comparing myocardial depressant factor (MDF) activity in SAO shock animals versus SAO Shock Sham animal (*P=0.065 compared to Sham shocked animals).

4.4 Discussion

This study shows that plasma from SAO shock activates neutrophils, as determined by NBT test and pseudopod activation. Although leukocyte activation has been observed previously in other shock models, its presence had not been identified in SAO shock. The observation that the activating factors are found in plasma indicates that this neutrophil activation is a systemic phenomenon, and the finding of the factors in SAO shock points to the splanchnic region as their possible origin.

The identity of the neutrophil activators released in SAO shock and their mechanism of production is unknown. Different mediators such as interleukin-2 (Il-2) and tumor necrosis factor-α (TNF-α) as well as monocytes and lymphocytes have been reported to be activated during SAO shock. Platelet activating factor (PAF), a phospholipid released from damaged cell membranes, is also reported to be involved in this shock model and inhibition of PAF has been shown to be beneficial in SAO shock. (The role of PAF as a potential neutrophil activating factor is discussed in more detail in Example 9).

Although the factors that activate neutrophils in SAO shock are not conclusively determined, there is a body of evidence that suggests that the pathology encountered in this shock model, like that in hemorrhagic shock, may be related to leukocyte upregulation and inhibition of this activation, in the splanchnic region and systemically may have a protective effect. SAO shock is an ischemia/reperfusion injury model that targets predominantly the pancreas by clamping its two main supply arteries. lschemia/reperfusion studies have also been carried out on the pancreas alone, in which the arteries feeding specifically the pancreas (the gastroduodenalis, lienalis, gastrica sinistra and gastricae breves) are clamped. Clamping of these arteries for 60 minutes results in acute pancreatitis wit marked neutrophil accumulation but no reported mortality after 120 minutes. This is consistent with results from the studies here showing that 60 minutes of SAO shock is not necessarily lethal to the rat within a two hour reperfusion period (data not shown). In pancreatic ischemia/reperfusion injury, as in SAO shock, the “trigger event” for organ injury and subsequent death is appears to be the free radical burst initiated upon reperfusion of the pancreas and gut region which then leads to activation of leukocytes, endothelial cells, and subsequent liberation of lipid mediators, and pancreatic proteases. Inhibition of free radicals with superoxide dismutase (SOD) and catalase mitigate this injury in part by mitigating the production of PAF due to oxygen free radicals.

Alternatively, it has been proposed that the rapid sequelae of events occurring after the unclamping of the arteries is not predominantly a free radical-mediated event due to sudden reoxygenation of the tissue but rather caused by the sudden release of proteases and other toxic components from the pancreas into the general circulation. It has been pointed out for example, that such phenomena as leukocyte influx into the pancreas, pancreatic edema, and inflammation cannot occur until flow has been reestablished with the outside circulation. Regardless of the trigger mechanism, it is evident that hypoxia to the pancreas and the release of toxic mediators, either due to low-flow conditions or outright ischemia, is potentially lethal to the organism.

SAO shock is a selective ischemia/reperfusion injury that targets the splanchnic region in general and the pancreas in particular. This form of injury results in the upregulation of systemic cardiovascular cell activating factors as well as other harmful mediators such as myocardial depressant factor. These mediators arise from the pancreas, and may be more depressant on pancreatic injury than on ishcemia/reperfusion per se.

### EXAMPLE 5

### Localization of Neutrophil Activating Factors in Tissue

Summary

Hemorrhagic and endotoxic shock as well as shock following splanchnic arterial occlusion (SAO) result in upregulated levels of leukocyte activation. The activation causes pseudopod formation and nitroblue tetrazolium (NBT) tests in donor neutrophils exposed to shock plasma. To determine the origin of the responisible factor(s), homogenates were made of rat organs, which were then tested for activation of naive neutrophils. Rats randomly selected were weighed and anesthetized and arterial and venous catheters were inserted. A laparotomy was made and the animals were exsanguinated. Organs including the spleen, small intestine, pancreas, heart, and liver were immediately removed and put into 0.25 M sucrose solution pending homogenization. In two animals kidneys and adrenals were also collected. Organs were then homogenized in 1:3 (w/v) Krebs-Henseleit solution. After homogenization, the suspension was then further diluted with 1:2 (v/v) Krebs-Henseleit. Two aliquots of each homogenate were taken; one aliquot was stored at 4° C. while the other was incubated for 2.5 hours at 38° C. to determine whether endogenous tissue enzymatic activity would enhance release of activation factors. Both sets of samples were tested for neutrophil pseudopod formation and NBT activity. Results indicate a significant increase (P<0.001) in leukocyte activation by incubated pancreatic homogenate, as well as a smaller but significant (P<0.005) increase in non-incubated homogenate. Activation from all other organs was non-significantly elevated compared to control samples.

It was then determined that activation occurs in pancreatic homogenates of other species. In five male pigs randomly selected the pancreas was removed and put into a 0.25 M sucrose-saline solution pending homogenization. The organs were homogenized in 1:4 (w/v) saline solution. Samples were incubated for 2.5 hours at 38° C. and tested for neutrophil pseudopod formation and NBT activity. Results from both sets of tests indicate a significant increase (P<0.001) in leukocyte activation by incubated porcine pancreatic homogenate as compared to controls.

Demonstrations of the size of the rat and porcine pancreatic homogenate activator(s) are provided. Aliquots of rat and porcine pancreatic homogenate were ultra-filtered using a 3 kD cut-off. The non-filtered and the low-molecular weight fraction activate neutrophils (P<0.001), indicating that at least one of the in vitro neutrophil activating factors in rat and porcine pancreatic homogenate is a low molecular weight species. The results suggest that the pancreas serves as an endogenous source for neutrophil activator(s) in shock and in inflammatory conditions.

5.1 Introduction

The splanchnic region has been implicated as a possible precursor site for the formation of activating factors of neutrophils (PMNs)(see Example 4). Since whole-body hypotension, endotoxic shock and SAO shock involve ischemia in the splanchnic region, it is possible that low flow to this region is a common mechanism resulting in the formation of circulating neutrophil activators in shock.

Also, other investigators have identified a myocardial depressant factor (MDF), which depresses cardiac contractility and is released in response to hemorrhagic, endotoxic, and splanchnic arterial occlusion shock, in addition to other models (Glenn (1971) Circ Res 29:238-249). A major difficulty in isolating and identifying this compound was obtaining sufficient quantities of material to assay. Lefer et al. (see, eg., Lefer (1970) Am J Physiol 218:1423-1427; Lefer (1972) Am J Physiol 223:1103-1109) examined homogenates from different organs and found that it was possible, after mild incubation, to produce MDF ex vivo.

Because of the possible similarities between the production of MDF and the neutrophil activating factors isolated herein, the qustion of whether neutrophil activation observed in shocked plasma could be reproduced by incubated organ homogenates to activate neutrophils ex vivo, in order to spatially identify the origin of a neutrophil activating factor(s) and determine whether such a factor is endogenously present (preformed in the tissue as opposed to synthesized during the shock state) was examined.

Tissue samples of small intestines, spleen, pancreas and liver, heart, kidney, and adrenals were taken from anesthetized, exsanguinated rats, homogenized and incubated. They were then assayed for their ability to activate donor neutrophils using NBT and pseudopod formation tests. Porcine pancreases were examined to determine whether organ-induced neutrophil activation was a species-dependent phenomenon. To identify the molecular size of these neutrophil activators, pancreatic homogenates were ultra-filtered through a 3kD filter and assayed for activation.

5.2 Methods

The organ homogenization procedure was similar to the method of Lefer (Glenn et al. (1971) Circ Res 29:338-349, Lefer (1973) Am J. Physiol 224:824-831). Male Wistar rats (250-350 gm) were housed in a controlled environment and maintained on standard pellet diet for at least three days before initiation of experimental procedures. Animals were cannulated via the femoral arteries and vein under general anesthesia using phenobarbital (50 mg/kg i.m.) No heparin was injected other than that needed to ensure open catheter lines (10 U/ml plasma-Lyte). A central incision was made over the abdomen. The rats were exsanguinated and the heart, liver, spleen, small intestine, and pancreas were removed. In two animals a kidney and adrenal gland were removed as well. They were immediately washed and cleaned in cold 0.25 M sucrose solution. The cleaned organs were vigorously homogenized in Krebs-Henseleit solution (1:3 w/v). Homogenate was then further diluted in Krebs-Henseleit solution (1:2 v/v). Aliquots were filtered by centrifugation at 500 G for 10 min and a sample aliquot was stored at 4° C. until assayed. The other fraction was incubated for 2.5 hours at 38° C. with mild stirring and stored at 4° C. until assayed. Incubation of tissue homogenates for this time period at a moderate temperature enhances any enzymatic activity that may be necessary in order to produce a neutrophil activation product. The incubated and non-incubated organ homogenates as well as controls were tested against naive human donor leukocytes for pseudopod formation tests and on whole rat donor blood for the NBT assay. Pseudopod determination was carried out as described in Example 2 using isolated neutrophils in D-PAS combined with homogenate in a 4:1 ratio. Nitroblue tetrazolium reduction by leukocytes due to superoxide in rat homogenate assays was also tested using the NBT procedure described in Example 2, with the application of 25 μl of homogenate to donor blood. The NBT assays for porcine homogenate activity were measured using the slightly modified NBT protocol with the crystal violet stain, also detailed in Example 2.

In order to collect pig pancreas, Male Hampshire pigs, 3-4 months of age, weighing 18-20 kg were used. Animals were restricted from food for a period of 24 hours prior to surgery. Surgical anesthesia was induced with ketamine (33 mg/kg/IM) plus atropine (0.05 mg/kg/IM) and sodium thiopental (10 mg/kg/IV) and then maintained with a combination of 1-2% halothane and oxygen. Animals were euthanized with pentobarbital (120 mg/kg/IV) and the pancreas was immediately harvested and rinsed in cold saline.

The pancreas was transported on ice in a 1:1 saline:0.25 M sucrose solution. The pancreas was cleaned of fat and excess tissue, weighed and blended for five minutes in a commercial blender using saline in a 1:4 w/v ratio. Blended homogenate was vigorously shaken and incubated for 2.5 hours at 38° C., shaken every 15 minutes. Incubated homogenate was centrifuged for 30 minutes at 800 G and the supernatant passed through a 0.78 μm vacuum filter (Millipore Filter Co., Beverly, Mass.). Pseudopod formation was determined on human donor neutrophils using the method described in Example 2 with isolated neutrophils in D-PAS combined with homogenate in a 4:1 ratio.

To avoid potential contamination of samples and possible non-specific activation due to bacterial products, sample aliquots were randomly treated with a standard cell culture combination antibiotic-antifungal agent. (Antibiotic-Antimycotic (100×) containing 10,000 U/ml penicillin (base), 10,000 μ/g/ml streptomycin (base), 25 μg/ml amphotericin B in 0.85% saline (Gibco BRL catalog # 15240-013, Gibco, Grand Island, N.Y.)) at 0.5% concentration by volume. Concentrations of the agent in this range have been reported to have no effect on neutrophil function.

To examine possible low-molecular weight activity in rat pancreas, homogenate was filtered with a 100 kD MW cutoff using a fixed-rotor Amicon filter (Model S-100, Centricon, Millipore Filter Co., Beverly, Mass.). An aliquot was saved for pseudopod formation measurements and the filter effluent was then further distilled through a 3 kD MW cutoff, again using a fixed-rotor Amicon filter (Model S-30, Centricon, Millipore Filter Co., Beverly, Mass.). Non-filtered sediment was reconstituted to its original volume with Krebs-Henseleit solution. All ultrafiltered aliquots were kept at 4° C. until use. Measurements of neutrophil activation were made as described above. In subsequent experiments the 100 kD filtering step was omitted.

Low-molecular weight activity in porcine homogenate was processed in the same manner, omitting the 100 kD filtering process.

Low-molecular weight composition of homogenates was randomly verified by MALDI mass spectroscopy (Mass Spectroscopy Laboratory, The Scripps Institute and Research Foundation, La Jolla, Calif.).

Results are expressed as Mean +SD for all samples. A two-tailed unpaired Student's t-test was used for all comparisons. Differences with P<0.05 were considered significant.

5.3 Results

5.3.a Rat Homogenate Results

Isolated naive human neutrophils incubated for 10 minutes with filtered homogenate from different rat organs (n=6 for each organ, except for kidney and adrenals, n=2), displayed little activation as measured by pseudopod activation when not-incubated, except for pancreatic homogenate which significantly activated naive neutrophils (P<0.001 compared to controls and all other organs). Intestine homogenate was not measured for this particular test due to non-specific contamination of the samples.

Incubation of these same tissues (n=6 organs each) for 2.5 hours increased the level of neutrophil activation by the pancreatic homogenate to 97.2±3.4% (P<0.001 compared to controls and all other organs measured), but neutrophil activation by other tissue homogenates was not significantly different from either control values or non-incubated homogenate levels.

NBT assays for superoxide production (n=5 organs each) likewise resulted in minimal activation of neutrophils in rat whole blood mixed with incubated organ homogenates except for pancreatic homogenate with significantly activated (42.8±15%) neutrophil superoxide production (P<0.005) compared to controls) and incubated liver homogenate, which also resulted in significant superoxide formation (P<0.05 compared to controls) by this assay (20.3±4%). The pancreatic homogenate also induced significantly higher activation when compared with all other organ homogenates and NBT levels in response to incubated liver homogenate were not significantly different from other organ homogenates. Because of extremely low levels of neutrophil pseudopod activation activity (less than control values), kidneys and adrenal homogenates were not assayed for NBT superoxide production.

Neutrophil pseudopod formation after incubation in the ultrafiltered pancreatic homogenate with a molecular weight less than 3,000 D (as verified by mass spectroscopy) also resulted in significant levels of activation (57.6±10.8%) compared to control values (6.2±1.5%) (P<0.001), although activity was decreased to some extent compared to non-ultrafiltered homogenate. There were no significant differences in pseudopod activation between the low molecular weight (<3kD), mid-weight molecular weight fractions (<100 kD) (60.8±13%) and the whole pancreatic homogenate fraction (68±8.9%). The high molecular weight fraction (MW>100 kD) displayed significantly lower levels of activation (P<0.05) (41.7±8.8%) compared to whole pancreatic homogenate.

5.3.b. Pig Homogenate Results

Results from pseudopod assay tests after incubation with pig pancreatic homogenate (n=5) indicate that the low-molecular weight fraction (<3 kD) as well as the unseparated porcine pancreatic homogenate significantly activate (P<0.001 for both tests compared to controls) isolated human neutrophils. Whole porcine pancreatic homogenate percentage neutrophil activation was 86.2±5.5% while that of the low-molecular weight homogenate was 41.7±19.6%. The increase in neutrophil activation by the low-molecular weight fraction is also significantly lower than the whole homogenate sample (P<0.055), suggesting that the low-molecular weight fraction is possibly not as potent an actin polymerization activator in the pig as its counterpart in the rat.

Porcine homogenate, low molecular weight (<3 kD) as well as whole homogenate, also significantly activated neutrophils in whole blood as assayed by the NBT test (P<0.001 for both tests compared to controls) using the crystal violet stain for cell identification. In this test there was no significant difference between neutrophil activation by whole and low-molecular weight samples. As discussed in Example 2, some caution must be exercised when comparing results from the NBT test using this stain with the NBT test using the standard protocol (Wright stain), since the crystal violet stain provides significantly higher contrast, making visualization of NBT crystals easier. Apparent NBT(+) results are thus increased in activated samples.

Addition of the antibiotic-antifungal agent in the concentration range of between 0.05-10% vol/vol as no effect on either nominal neutrophil activation or neutrophil activation after exposure to pancreatic homogenates as measured by pseudopod formation. In higher doses (100× the recommended concentration) there is a small dose-dependent activation.

5.4 Discussion

This study was designed to determine whether neutrophil activators are produced in selected tissues of the body. Such factors could be released into the circulation in shock or other traumatic conditions and contribute to leukocyte activation and subsequent global injury. While not examined here, the same activator may also affect other cells in the cardiovascular system (see Example 2 for in vivo observations). It was hypothesized that cellular activators are produced upon tissue injury, perhaps in response to oxygen free radicals. Examples of these include platelet activating factor (PAF), tumor necrosis factor-α (TNF-α) and other cytokines. It had however, not been conclusively demonstrated that these known activators are responsible for the initial neutrophil activation seen in vivo. It was of interest to deterimine whether such factors are endogenous to one tissue or formed globally by ubiquitous cell types such as endothelium and macrophages. Cytokines and bioactive lipids (e.g., PAF) may be produced in a number of organs but may be specific to certain cell types.

The results provided herein point to a particular role for a single organ the pancreas in the production of neutrophil activators. In view of the finding that neutrophil activators circulate during shock following splanchnic arterial occlusion (see Example 4) as well as in response to hemorrhagic and endotoxic shock, special attention was given in these studies to organs of the splanchnic region. These organs are known to be susceptible to ischemia and other manifestations of shock (e.g., acidosis, bacterial translocation). The pancreas has been shown to be particularly sensitive to not only local but global ischemia, such as hemorrhagic shock. Ischemia to this organ may result in the production and release of circulating activating factors.

As noted above, others, (Lefer et al. (1973) Am J. Physiol 224:824-831, Lefer et al. (1970) Circ Res 26:59-69) have isolated a myocardial depressant factor (MDF). Since the current results of the SAO shock studies demonstrate the presence of MDF as well as neutrophil activation (see Example 4), MDF might be a neutrophil activating factor. Even if MDF is not a neutrophil activating factor, there exists the possibility that an endogenous neutrophil activating factor might be produced in the same manner or even co-localized with MDF. This supposition is not likely.

MDF has been postulated to be a peptide attached to a long-chain fatty acid, having a putative molecular weight of 800-1,000 daltons and this could be a potential low-molecular weight neutrophil activator. Contrary to results reported here in which even non-incubated pancreatic homogenate strongly activates neutrophils (albeit to a lesser degree than incubated homogenate), however, little MDF formation without homogenate incubation was found, suggesting that MDF is not constituitively present but is formed via an enzymatic degradation process.

In contrast production of the pancreatic neutrophil activating factor provided herein does not appear to be highly dependent upon enzyme function. The implications of this finding are that the pancreatic neutrophil activating factors are either preformed moieties that are released upon cell disruption or ischemia, or are formed during shock independently from enzymatic processes. The latter supposition would eliminate small pancreatic peptides as possible neutrophil activators, as these tend to be degradation products formed from larger (pro-enzyme) amino acid chains. Alternatively, small lipids, either preformed or released in response to oxidative stress, cell disruption, or ischemia may function as pancreatic neutrophil activators.

Results from actin polymerization and NBT reduction (see also Example 9 for neutrophil superoxide production results obtained with a chemiluminescence method) are consistent, identifying the pancreas as the only homogenate tissue source resulting in in vitro neutrophil activation in the rat. This finding was surprising, since the intestine, as well as other gut organs which have been implicated in in vivo neutrophil activation, yield little neutrophil activation.

An alternative interpretation of these results is that all organs contain factors that can activate neutrophils but the pancreas is the only organ among those studied here that does not contain a neutrophil inhibitory factor. The pancreas, as the main organ of exocrine and digestive enzymes in the body is somewhat unique among other organs and may be the site for enzymatic digestive processes. It is interesting, however, that other studies that have found that homogenate from some tissues including the thymus, intestine, spleen and heart do contain a neutrophil inhibitory substance that actually decreases neutrophil activation compared to controls in a dose-dependent manner. In no reported case has the pancreas been analyzed. The inhibition has been confirmed by studies described below (Example 7).

Results from neutrophil activation tests on porcine pancreas agree with those obtained from rat pancreas experiments. Pseudopod formation and NBT tests on neutrophils incubated with porcine pancreas result in significant levels of activation, compared to control samples. A discrepancy exists, however, in the results with the low-molecular weight porcine homogenate. Low-molecular weight pseudopod formation tests indicate that actin polymerization activity, although significantly greater than in control samples, is also significantly less than in whole porcine pancreatic homogenate. The test with the rat and the pig agree in this respect. NBT tests, however, show comparable activation of neutrophils incubated with either low-molecular weight or whole porcine pancreatic homogenate. These findings are consistent with results from neutrophil lucigenin-enhanced superoxide production (see Example 6), which demonstrates similar activation in whole and low-molecular weight fractions. The reason for this discrepancy is unclear. As discussed in Example 2, different kinds of neutrophil activation such as pseudopod formation, superoxide production and ligand shedding tend to be uncoupled as a rule. It is possible that the low-molecular weight pancreatic neutrophil activators present in the pig might preferentially activate the NADPH oxidase pathway and less strongly, actin polymerization.

Results from antibacterial-antimycotic administration indicate that the presence of these compounds in the concentrations given have little effect on neutrophil activation, either in control or activated samples. Due to the fact that it is almost impossible to prepare a truly sterile homogenate, especially in the pig, administration of an antibacterial-antimycotic agent as a prophylactic measure may serve to guard against bacterial contamination and non-specific neutrophil activation. Neither can bacteria be filtered through the low-molecular weight cutoff filters, not has mass spectroscopy yielded any peaks corresponding to the bacterial chemotactic peptide fMLP in any of the samples studied.

This study served to discover endogenous in vitro neutrophil activators that occur in pancreatic homogenates and not in homogenates of other organs. The finding of analogous neutrophil activation factors in porcine pancreas indicates that the pancreas produced neutrophil activating factors may not be exclusively specific to the rat. In view of the finding that the related low-molecular weight substance MDF, also produced by the pancreas, occurs in man as well as a number of experimental animals, it is highly likely that the pancreas-produced neutrophil activating factors are formed in a variety of animal species, including man.

The results from this study point to the existence of at least one low-molecular weight neutrophil activator emanating from the pancreas, but do not preclude the presence of other higher molecular weight (20-40 kD) activators, such as proteases. The pancreas is a unique organ in the body in that it possesses a wide range of digestive enzymes and other potentially inflammatory compounds. It is possible that there exists a synergy in the whole pancreatic homogenate between larger proteases and the low-molecular weight activator.

### EXAMPLE 6

### In Vitro Chemiluminescence Measurements of Plasma Superoxide Production by Pancreatic Activating Factors

Summary

Activtors for blood cells in the circulation are currently not well identified in shock. As shown herein, a pancreas homogenate and not other organs studied (heart, liver, spleen, intestine, adrenals, kidney) will activate naive donor neutrophils, as measured by pseodopod formation.

Whether neutrophil activation could be detected by lucigenin chemiluminescence due to superoxide production and whether chemiluminescence could be detected from pancreatic homogenate of another species, in this case the pig, was studied. The pancreas of six rats were homogenized in 1:9 (w/v) Krebs-Henseleit buffer, incubated for 2-5 hours at 38 C and aliquots filtered with a 3 kD cutoff. In analogous experiments the pancreas was removed of five male pigs and put into a 2.5 M sucrose-saline solution pending homogenization. The organs were homogenized in 1:4 (w/v) saline solution. These samples were incubated for 2.5 hours at 38 C and aliquots were filtered with a 3000 MW cutoff and tested for chemiluminescence. Samples were measured for superoxide chemiluminescence in human donor plasma. Results from rat homogenate indicate a significant increase (P<0.001) in superoxide induced chemiluminescence by pancreatic homogenate, in the non-filtered and the low-molecular weight fraction. Results from the porcine experiments also indicated a significant increase (P<0.001) in superoxide induced chemiluminescence by pancreatic homogenate, non-filterd and the low molecular weight fraction versus control plasma. These results indicate that porcine pancreas, like rat pancreas, contains factors that activate neutrophils in vitro, including a low-molecular weight activator.

In other experiments chemiluminescence of isolated human neutrophils was measured in varying concentrationsx of autogolous plasma, with and without the addition of pancreatic homogenate and known stimulators fMLP and PAF. Results indicate temporal and spatial differences in superoxide production due to these activtors as well as the buffering effect of plasma. Pancreatic homogenate in the concentrations used provokes a much greater superoxide repsonse than either fMLP (10⁻⁶ M) or PAF (10⁻⁶ M).

The purified pancreatic homogenate activated other cell types In vitro in addition to neutrophils. To study this possibility, chemiluminescence tests were conducted using plated bovine aortic endothelial cells (BAECs) subjected to pancreatic homogenate, low-molecular weight pancreatic homogenate, or control solutions. Results indicate a significant increase (P<0.001) in chemiluminescence in BAEC cultures incubated with whole pancreatic homogenate. Low-molecular weight pancreatic homogenate-induced activations was not significantly greater than control values. These results indicate that pancreatic homogenate contains factors that activate endothelial cells in vitro. Factors in pancreatic homogenate may be powerful endogenous activators of neutrophils and endothelium in inflammatory conditions.

6.1 Introduction

There are endogenous factors capable of upregulating neutrophils in vitro and that these factors are located in the pancreas but not in other tissues (see Example 5). The factors are capable of upregulating neutrophils in vitro as measured by pseudopod formation and NBT tests. As lucigenin chemiluminescence has been used as an assay to measure superoxide formation in leukocytes, it was of interest to find whether or not factors in pancreatic homogenate would activate quiescent donor neutrophils as measured by this test.

Since the pancreas-derived neotrophil activating factors are present in species other than the rat, the opportunity exists to obtain sufficient quantities of crude extract for subsequent purification of these factors.

In addition to studying neotrophil enhanced chemiluminescence in response to pancreatic homogenate it was also of interest to determine whether pancreatic homogenate would have superoxide eliciting properties on other types as well. Because the endothelium plays a predominant role in neutrophil activation and adhesion has been implicated as a major source of superoxide production, the effect of the pancreatic homogenate applied to endothelial cell cultures superoxide production was studied.

Finally, it was of interest to study the differences in neutrophil activation as measured by chemiluminescence due to these factors, in the presence and absence of plasma, and compared to other well-studied activators of neutrophils. The interaction between neotrophils and the plasma component of blood is an important, often overlooked factor in assessing presence and severity of different disease pathologies. One method of measuring neutrophil activation is by chemiluminescence using lucigenin, luminol, or other chemiluminescent compounds which amplify photons produced upon neutrophil production of oxygen free radical intermediates and other reactive products. (Delong et al. (1989) J Chromatogr 492:319-343; Ginsburg et al. (1993) Inflammation 17:227-243).

Among the species of interest produced by activated neutrophils in the circulation is superoxide anion (O₂⁻). Many methods are currently in use to detect this oxidative species, including NBT, cytochrome C, luminol and phenol red tests. Lucigen-enhanced chemiluminescence is another such method. Unlike luminol-produced chemiluminescence, which is a relatively nonspecific marker for superoxide, hydrogen peroxide as well as myeloperoxidase, lucigenin reacts specifically with superoxide to produce light. Lucigenin (dimethyl diacridinium nitrate) reacts in a two-step reaction (see, e.g., Faulkner et al. (1993) Free Radic Biol Med 15:447-451) primarily detects superoxide (see, eg., U.S. Pat. No. 5,294,541). Lucigenin chemiluminescence can be quenched by superoxide dismutase (SOD), an enzyme specific for superoxide, and otherwise reacts as a specific measure of membrane-bound NADPH oxidase produced superoxide.

Although frequently chemiluminescence tests are carried out on isolated leukocytes or other cells, in inflammatory conditions a large percentage of activated neutrophils are necessarily adherent to the vascutiure and thus neutrophils collected from sampled blood may not accurately reflect the actual degree of activation. Conversely, many inflammatory mediators circulate in the blood and therefore it is the non-cell component which is perhaps a more reliable indicator of heatlh. It it thus possible that plasma collected during inflammatory conditions offers a more accurate assessment of clinical severity than isolated neutrophils per se. Collection of plasma is an easier process than neutrophil isolation, and may avoid possible artifacts that can occur with the handling and isolation of neutrophils. It has been shown that autologous plasma potentates the response of neutrophil-induced chemiluminescence in vitro (Theron et al. (1994) Inflammation 18:459-567), so it was also of interest to determine whether or not collected plasma would serve as a viable alternative to isolated neutrophils in measuring superoxide chemiluminescence.

Lucigenin-produced chemiluminescence as a means to measure concentration in plasma was studied. Plasma measurements have the advantage over isolated cells (e.g., neutrophils) because they are two-step methods (centrifuge and measure), amenable to large numbers of measurements and automation. Chemiluminescence produced by isolated human neotrophils at varying concentrations of autogolous plasma, with and without standard activators (fMLP and platelet activating factor), was compared. In addition, the homogenate activator from the rat and pig pancreas was tested to gain comparative understanding as to their temporal chemiluminescence activation properties in comparison with the known activators, rat and pig homogenate were ultracentrifuged in order to separate a low molecular weight fraction (<3kD) and measure separately its ability to activate neutrophils.

6.2 Methods

6.2.a Specifications

Human blood was collected in heparinized Vacutainer tubes (10 ml) and centrifuged at 500 G for 10 minutes. Plasma was collected and 3 ml were mixed with lucigenin (N,N′-dimethyl-9,9′-bisacridinium dinitrite) (Sigma Chemical CO., St. Louis, Mo.) (1 ml of a 1 mM stock solution in deinozed water, final concentration 200 μM, near optimal concentration as determined by Ohoi, et al (26)) for each measurement in small petri dishes (60 mm diameter). In later modifications of the test, a 25 mm diameter polyurethane disk was placed inside the petri dishes to reduce the vessel diameter, and subsequently, the reagent requirements to 1 ml plasma mixed with 0.75 ml lucigenin (1 mM stock solution) and only 100 μl of an activator. This smaller scaled version resulted in only minimal loss of signal and was used when either the activators were of a minute volume and concentration (such as rat plasms collected before shock protocol) or the number of measurements necessitated a large amount of autologous donor plasma and it was desired to apply the same plasma for each measurement. Six Vacutainer tubes were normally collected from healthy volunteers. This volume gives approximately 8 measurements using the original configuration (3 ml plasma/measurement) and up to 25 measurements with the modified system (1 ml/measurement). When measuring whole blood samples, 3 ml of whole blood were subsitituted for 3 ml of plasma.

6.2.b Neutrophil Isolation

A standard neutrophil isolation procedure was used as described in Example 2 or pseudopod formation tests. Human blood from healthy volunteers (approximately 60 ml) was collected in heparinized Vacutainer tubes and transferred to a 60 ml syringe where it was sedimented on ice for 40-60 minutes. It is important that heparin and not EDTA (ethylamine diaminetetraacetic acid) be used as an antocoagulant, since the calcium-chelating properties of EDTA can suppress neutrophil activation. The neutrophil-rich plasma layer was collected and layered onto 3.5 ml Histopaque (Sigma Diagnostics, St. Louis, Mo.) in 12 ml polypropylene centrifuge tubes (17×100 mm, Falcon, Shrewsbury, Mass.) and centrifuged at 600 G for 20 minutes. The sedimented neutrophils and red blood cells were then gently resuspended in 2 ml PBS (phosphate buffer solution). The resuspeneded cells were carefully layered onto 2.5 ml of a 55% isotonic Percoll solution (Sigma Diagnostics, St. Louis, Mo.) and 2.5 ml of a 74% isotonic Percoll solution in deionized water. This suspension was centrifuged at 600 G for 15 minutes and the middle granulyte layer was removed and resuspended in PBS to achieve a concentration of 10⁶ neutrophils/ml.

For standard plasma measurements (e.g., low molecular weight fractions and whole pancreatic homogenates) human blood from healthy volunteers (approximately 60 ml) was collected in heparinized Vacutainer tubes and centrifuged for 10 minutes at 500 g. The plasma layer, including the buffy coat was carefully decanted using a steril transfer pipette. This fraction was then warmed to room temperature and plasma measurements were obtained. Plasma was diluted with sterile saline to achieve a plasma neutrophil concentration of 120×10³ neutrophil/ml.

For experiments with different plasma concentrations versus neotrophil activators, measurements were made using isolated neutrophils, isolated neutrophils in 10% and 30% autologous plasma, and neutrophil-free plasma (filtered with 1 um filter and verified by cell count).

6.2.c Activators

Cell-free plasma was obtained by filtering sedimented plasma through a 1 um filter (Whatman 6780-2510, Swedesboro, N.J.). The filtered plasma was visually verified to be cell free by placing an aliquot on a microscope slide and examining in detail at 400×. For experiments with different percentages of added plasma, 5 ml total volumes were made consisting of 1 ml lucigenin (1 mM), 1 ml activator, and either 3 ml suspended neutrophils, 2 ml suspended neutrophils plus 1 ml cell-free plasma mixed in PBS necessary for either 10% or 30% total plasma concentration, or 3 ml cell-free plasma. Activators (1 ml) were added via an injection port to the mixture at time t=0 seconds. The activators used were pancreatic homogenate, whole and low MW fraction, chemotactic peptide N-formyl-Methionyl-L--Leucyl-L-Phenylalanine (fMLP)(10⁻⁶M) (Sigma Chemical Co., St. Loius, Mo.), and platelet activating factore (PAF)(10⁻⁶)(Sigma Chemical Co., St. Louis, Mo.). 1 ml PBS served as the control activator. Superoxide dismutase from bovine erythrocytes was obtained from Sigma Chemical Co., St. Louis, Mo.

Rat pancreas homogenate was prepared as previously described. Briefly, the pancreas from male Wistar rats, 3 months of age, weighing 250-250 g were harvested and rinsed in a cold 0.25 M sucrose solution, cleaned of fat and excess tissue, weighed and blended for fifteen minutes using a homogenizer in Krebs-Henseleit solution 1:3 w/v ratio. The mixture was then futher diluted with Krebs-Henseleit solution in a 1:2 volume homogenate/volume ratio and incubated for 2.5 hours at 38 C, shaken every 15 minutes. Incubate homogenate was centrifuged for 30 minutes at 800 G. The filter effluent was filterd with a 3,000 MW cutoff using a fixed-rotor Amicon filter (Model S-30, Centricon, Millipore Filter, Co., Beverly, Mass.). Ultrafiltered aliquots were kept at 4 C until use.

For collection of porcine pancreatic homogenate, male Hampshire pigs, 3-4 months of age, weighing 18-20 kg were used. Animals were restricted from food for a period of 24 hours prior to surgery. Surgical anesthesia was induced with ketamine (33 mg/kg/IM) plus atropine (0.05 mg/kg/IM) and sodium thiopental (10 mg/kg/IV) and then maintained with a combination of 1-2% halthane and oxygen. Animales were euthanized with pentobarbital (120 mg/kg/IV) and the pancreas was immediately harvested and rinsed in cold saline. All pig experiments were done by Dan McKirnan at the UCSD Elliot Field Station in accordance with University of California, San Diego Animal Subjects Committee regulations and requirements.

The pancreas was stored and transported on ice in a 1:1 saline:0.025 M sucrose solution. The pancreas was cleaned of fat and excess tissue, weighed, and blended for five minutes in a commercial blender using saline in a 1:4 w/v ratio. Blended homogenate was vigourously shaken and incubated for 2.5 hours at 38 C, shaken every 15 minutes. Incubated homogenate was centrifuged for 30 minutes at 800 G and the supernatant passed through a 0.78 um vacuum filter (Millipore Filter Co., Beverly, Mass.). Filter effluent was then further filtered with a 3,000 MW cutoff using a fixed-rotor Amicon filter (Model S-30, Centricon, Millipore Filter Co., Beverly, Mass.). Ultrafiltered aliquots were kept at 4 C until use. MALDI mass spectroscopy measurements of selected samples verfied that no signal was detected above 3,000 MW. In fact, no signal could be detected above 1,000 MW.

6.2.d Measurements

The resulting photon emitted from the generated chemiluminescence were counted for a period of not less than 120 minutes with a photomultiplier tube (using a light accumulation period of 1 second) (Stanford Research 4000, Sunnyvale, Calif.) encased in a light-shielded apparatus and connected to a PC computer (486 Dell Computer Corp., Austin, Tex.) for data storage (SR467 Data Acquisition Software Package, Stanford Research Systems, Inc., Sunnyvale, Calif.). The photon counter and system was provided by Mr. Richard Suzuki, from the Department of Bioengineering, Univeristy of California, San Diego, with minor modifications in experimental technique.

Chemiluminescence experiments were made either serially, when either recording of an entire time history was required (such as in the case of assay curves with sharp spikes in amplitude), or in batch mode, where several samples were rotated (manually) throughout the experiment. The batch method was traditionally used, since it avoids a possible degredation of effect of plasma and other biological materials whihc may have occurred if the experiments were carried out sequentially. In the batch mode, up to 12 samples at a time were measured at intervals of appoximately five minutes. In less temporally-dependent experiments, measurements were spaced out up to every 10 minutes.

6.2.e Endothelial Cell Culture Measurements

Experiments of endothelial cell chemiluminescence response to pancreatic homogenate were made using confluent bovine arterial endothelial cells (BAECs), (Department of Bioengineering, University of California, San Diego). BAECs were grown in 60 mm diameter petri dishes at 37 C in a controleed cell culture environment, incubated in standard RPMI medium (Gibco, Grand Island, N.Y.). For chemiluminescence measurements, cell cultures were surveyed by light microscope for confluence and rinsed two times with standard Krebs-neseleit buffer to eliminate possible optical effects of residual media. One ml Krebs-Henseleit buffer was added to the culture followed by 1 m 10⁻³ lucigenin. For pancreatic homogenic experiments, either 1 ml of whole pancreatic homogenate or 1 ml of low-molecular weight pancreatic homogenate was added. 1 ml of Krebs-Henseleit solution was added to control cultures. Chemiluminescence was measured as describe above using a 1 minute light accumulation period. All endothelial chemiluminescence tests were done in duplicate.

6.2.f Chemiluminescence System Calibrations

Calibration for the lucigenin chemiluminescence assay was performed by adding lucigenin to 3 ml buffered saline solution. Known concentrations of potassium superoxide (KO₂) (Sigma Chemicals, St. Louis, Mo.) which spontaneously decays into superoxide and K+ in aqueous solution were added and the chemiluminescence was measured. A linear response was obtained between 1 nM and 10 μM. A potassium superoxide curve is preferable as a calibration of superoxide as KO₂ spontaneously reacts to form superoxide in a 1:1 ratio (allowing a direct quantification of absolute concentrations of superoxide) while xanthine oxidase produces varying levels of superoxidase and H₂O₂ depending on experimental conditions

As noted above (Example 3) Xanthine (and hypoxanthine) react with xanthine oxidase to produce superoxide and hydrogen peroxide. Under quiescent conditions, xanthine oxidase exists as a xanthine dehydrogenase and reacts with NAD⁺ to form NADH and uric acid.

It is included as a common indicator of relative amounts of free radicals produced and to facilitate comparison with results by other groups. Hydrogen peroxide does not react with lucigenin and no signal is obtained (curve not shown). DMSO (dimenthyl sulfoxide) should not be used as a reagent since it reacts strongly to produce chemiluminescence in plasma.

Results are expressed as Mean=/−SD for all samples, except for continuous chemiluminescence measurements which the standard deviation is omitted for readability. A two-tailed unpaired Student's t-test was used for all comparisons. Differences with a P<0.005 were considered significant.

6.3 Results

For a representative time course of control plasma without addition of an activator, the mean maximum (steady-state) values for each such experiments (n=100+) are approximately 3300+/−500 counts/sec in approximately 40 minutes. The time course is characteristic of lucigenin-measured chemiluminescence and appears to be related to ineractions between neutrophils and luceigenin.

Results from chemiluminescence tests of low-molecular weight (n=6) as well as whole rat pancreatic homogenate (n=6) show a significant increase (P<0.05 to P<0.001) in pancreatic homogenate-treated samples compared to controls (n=3) by 40 minutes into the experiment. Plasma chemiluminescence (PMN concentration 150×103/ml) of whole rat pancreatic homogenate (n=6) and low molecular weight (<3 kD) rat pancreatic homogenate (n=6) versus controls (n=3). Control and activated samples show a typical increase in chemiluminescence during the time course of the experiment. It is unclear whether or not this increase is due to a loading phenomenon or posssibly a diffusion of the lucigenin in an otherwise well-mixed samples. Results from pancreatic homogenate added to cells alone are contrary to this. Results from plasma experiments after the addition of the whole-fraction of rat pancreatic homogenate (1 ml of supernatant solution as prepared above, approximately 11 μg total protein/μl) are much different from the preceding curves in that there is an enormous increase in superoxide-produced chemiluminescence even in isolated neutrophils

Application of the low molecular weight (n=5) and whole porcine pancreatic homogenate (n=5) to plasma also result in significant chemiluminescence (P<0.05-P<0.005) compared to controls (n=5). The results from control experiments of neutrophils alone (in PBS), neutrophils in 10% analagous plasma, neutrophils in 30% analagous plasma, and plasma alone were performed. Neither unstimulated neutrophils alone nor plasma without cells result in appreciable levels of chemiluminescence when taken from healthy donors. This may change in inflammatory conditions, not only with the resultant activated neutrophils but also with circulatin superoxide donors (such as circulating xanthine oxidase) which may be present in samples of plasma without cells.

The results from experiments made with human neutrophils alone, neutrophils in 10% autogolous plasma, neutrophils in 30% autogolous plasma and plasma alone after stimulation with the peptide activator fMLP (1 ml of 10⁻⁶M) showed that even large concentrations of this agent do not intrinsically activate NADPH oxidase. With increasing levels of plasma concentration there is a noncomitant increase in superoxide production. With plasma alone, there is very little superoxide production, as would be expected with the application of fMLP in a cell-free medium. Again, in inflammatory conditions with the presence of circulating activators in appreciable quantities this may not hold true.

The results from analogous experiments after the addition of platelet activating factor (1 ml of 10⁻⁸) showed that the general trend of these curves is to follow that of the other receptor-mediated activator, fMLP but at slightly lower levels.

The results from plasma experiments after the addition of the whole-fraction rat pancreatic homogenate (1 ml ug total protein/ul) show that these curves are much different from the preceding curves in that there is an enormous increase in superoxide-produced chemiluminescence even in isolated neutrophils, as well as differing levels of plasma. There is also a slight increase in chemeluminescence from plasma-only samples incubated with the pancreatic homogenate.

Incubation of rat pancreatic homogenate, low molecular weight as well as whole homogenate, resulted in a dramatic increase in chemiluminescence when applied to confluent bovine aortic enothelial cell (BAECs) cultures. Control BAEC cultures show demonstable increase in chemiluminescence in time, which was attributable to temperature sensitivity of the photomultiplier tube which displays slight increases in basal photon count as the instrument warms. This increase is less than 1% of normal measurement values when measured at a 1 sec photon-accumulation period but becomes appreciable when measuring the much lower values of endothelium-produced chemiliminescence measured at a 1 minute photon-accumulation period. Therefore, in all endothelial cell culture experiments this tempurature was controlled by precisely timing the length of machine warming. Results of control experiments differed by an average of 962+/−187 photons/minute (approximately 3-5% of total control sample photon counts).

6.4 Discussion

Neutrophil activation can be quantified with many different methods, such as NBT, pseudopod formation, and chemiluminescence, each of which measures a specific parameter of cellular response to a stimulus. Pseudopod formation, for example, is a measure of the actin polymerization that occurs when neutrophils respond to some chemotactic activator. Other responses of neutrophil activation include the upregulation of the NADPH oxidase system and subsequent production of oxygen-free radicals and the degranulationof the primary and secondary granules. Although these are all responses of activated neutrophils, they need not be coupled; different activators preferentially activate different conponents of the neutrophils cytoplasm and membrane. Since superoxide-induced chemiluminescence is a more “impartial” measure of neutrophil activation that does not require operator judgement (in contrast to NBT and pseudopod formation), it was determined whether the pancreatic homogenate that activates neutrophils as viewed by pseudophod formation and the NBT tests would also do so via lucigenin chemiluminescence.

This study showed that incubated homogenate from the rat pancreas as well as pig activates neutrophils, as determined by in vitro superoxide chemiluminescence production. Other tissue homogenates did not activate in vitro. The homogenates from the rat and the pig were prepared in analagous fashion, and have roughly equal strength, although the porcine homogenate is less diluted. These resuls point to the possibility of a common factor that is not species dependent. In the rat and the pig there was considerable chemiluminescence in the whole pancreatic homogenate as well as in the low-molecular weight fraction. Although the whole pancreatic homogenate contains the low-molecular weight fraction it had been hypothesized that any neutrophil activators emanating from the pancreas would be protease in orgigin, with molecular weights between approximately 30 kD and up to ove 100 kD (see Example 5). The findings that the rat and the pig contain a low-molecular weight (3<kD) component that activates NADPH oxidase production in neutrophils does not negate this view; larger molecular weight proteases have been shown to modulate neutrophil response to other activators and are probably synergistic in their responses. A low-molecular weight activator was unexpected, and points to the presence of a small peptide-like or lipid substance in the pancreas that mau endogenously activate neurophils. The relative strength of the low molecular weigth activators is at least as great as that of the entire molecular weight fraction, suggesting that for the activation of NADPH oxidase-produced superoxide the low molecular weight fraction is of primary importance. This is somewhat at variance with the data on pseudopod formation, which indicated that the whole pancreatic homogenate is invariably slightly more powerful than the low molecular weight fraction in promoting actin polymerization. Again, it is noted that the processes are not coupled.

In addition to the superoxide-induced chemiluminescence actions by pancreatic homogenate on neutrophils, pancreatic homogenate also activated endothelium in vitro as assayed by lucigenin chemiluminesnence. The relative difference in strengths between the whole pancretic homogenate, which activated very strongly over the course of one hour, and the low-molecular weight fraction, which activated much more weakly over that time period, is much different from that seen in neutrophil chemiluminescence studies. While neutrophil activated chemiluminescence appears to be present equally in whole and low-powered molecular fractions, results from the endothelial activation experiments imply the presence of high molecular wieght endothelial activators only. In neither case is the presence of high and low molecular weight activators precluded. A probable source for high molecular weight endothelial activators are the pancreatic (serine) proteases, which are discussed in greater detail in Example 7.

The observations of the relative strengths of the different activators necessary to upregulate the NADPH oxidase system in different concentrations of plasma were interesting. FMLP added to isolated neutrophils is not particularly reactive in terms of superoxide-induced chemiluminescence production. PAF, another receptor-mediated neutrophil activator is likewise unreactive in suspended neutrophils. Both agents were also unreactive in cell-free plasma. The addition of varying amounts of plasma to neutrophil cell suspensions greatly augmentd superoxide induced chemilumimescence, consistent with the conclusion that neutrophils require adequate extracellular ATP in order to mount a respiratory burst.

The results of the pancreatic homogenate chemiluminescence repsonse were particularly surprising. Although the homogenate does not possess any intrinsic chemiluminescence stimulating properties, the addition of homogenate to cell-free plasma results in a slight increase in chemiluminescence, something not seen with the other activators studied. More surprising was the result that pancreatic homogenate added to suspended neutrophils alone results in a dramatic and instantaneous increase in superoxide induced chemiluminescence. It is apparent that the homogenate in the concentrations used is an enormously potent activator of human neutrophils in vitro. It is perhaps possible that there exists some ATP-generating substances in the pancreas homogenate that can mimic those in autogolous plasma. Alternatively, differences in the NADPH oxidase activation pathway may be involved, such as is the case with the non-receptor dependent activator phorbol ester PMA which activates the superoxide dependent chemiluminescence in the absence of plasma. The homogenate, thus, will be very useful in assays for screening for inhibitors of its activity(ies).

The combination of a low-molecular weight stimulus with a high-molecular weight priming agent (such as serine protease which can cleave the CD41 ligand directly) may alleviate the need for the addition of plasma. The addition of 10% plasma greatly potentates the response of the isolated neutrophils to pancreatic homogenate. The magnitude of chemiluminscence derived from isolated neutrophils mixed with 10% plasma and activated with pancreatic homogenate were on average an order of magnitude greater than any activation produced by either fMLP or PAF. The time course of chemiluminescence was also retarded by the addition of plasma, i.e., increasing the plasma:neutrophil ratio, appears to decrease the superoxide-dependent chemiluminescence. It is possible to attibute this decrease in chemiluminescence to the prevalent antiprotease screen present in healthy plasma and a large percentage of the chemiluminescence due to the protease fraction of the homogenate. When one observes the relative magnitude of whole and low-molecular weight superoxide-induced chemiluminescence it is evident that protease-antiprotease interactions cannot be the sole consequences of increasing plasma concentrations. Separate chemiluminescence experiments with larger concentrations of the main pancreatic protease trypsin (1000 U/mI) and chymotrypsin (100 U/ml) added to the plasma (neutrophil concentration 155×10³ neutrophils/ml plasma) yielded little activation.

A final consequence of these chemiluminescence measurements was the establishment of a viable method for assaying superoxide prodcution ex vivo. As mentioned above, most current lucigenin chemiluminescence methods use isolated neutrophil from patients, which are then stimulated with a known stimulator such as PMA, and the resulting chemiluminescence measured. This type of approach has several drawbacks. Among these is the necessity for isolating neutrophils, which is time consuming, requires addition reagents, and more importantly, is subject to differences in the activation of neutrophils (which have already been centrifuged at lease twice and possibly subjected to different osmolarities if sedimented with dextran) to some stimulus which itself may have differences in potenecy. In addition, as mentioned above, neutrophils in individuals suffering from inflammatory conditions are already activated and the venous sampling of blood from such patients does not necessarily lead to an accurate measure of the percentage of activated cells, as activated neutrophils tend to become adherenet to the endothelium in the microcirculation and are not likely to be recovered in venous samples.

The method used in the studies herein alleviates the difficulties of the aforementioned assays by being simple, quick, repoducible, and inexpensive. It can be used in the classical fashion; that is, fresh patient blood is centrifuged and the plasma measured for superoxide formation. More often, control plasma from healthy individuals can be used as a vehicle to test activation of different substances, even other patient plasma. This latter method provides neutrophils in autogolous plasma and obviates the need for large amounts of patient plamsa. As little as 100 μl of plasma (and possible less using the new smaller volume configuration) can be measured for its ability to activate otherwise quiescent neutrophils. This method can give accurate results in as little as 1 hour (10 minutes centrifugation, 10 minutes setup and 40 minutes of measurement). Becauase the number of neutrophils in spun plasma is much less than that of isolated neutrophils in autologous plasma, the relative levels of chemiluminescence are likewise attenuated. In normal (control) plasma, all values thus far (>1 00 experiments with more than 5 different donors) have had a maximum repsonse of between 1500 and 6000 counts/sec ina time frame of 20-50 minutes. The normal range is approximately 3000+/−500 counts/sec in approximately 40 minutes. This can be modified by donor illness, antibiotics, and more interestingly, ingestion of fatty diet.

Low-molecular weight and whole fractions of pancreatic homogenate significantly increased superoxide produced chemiluminescence from the donor neutrophils and plasma compared to control values. In addition, whole pancreatic homogenate significantly increased superoxide production by BAEC endothelial cell cultures. Chemiluminescence activation produced by neutrophils and plasma incubated with pancreatic homogenate (9:1 vol/wt) was significantly greater than that expressed by comparable volumes of known activators fMLP and PAF, demonstrating that there may exist powerful factors in the pancreas that are capable of activating neutrophils and other cardiovascular cells.

### EXAMPLE 7

### Protease Involvement in Pancreatic Neutrophil Activators

Summary

Splanchnic arterial occlusion (SAO) shock results in upregulated levels of neutrophil activation, as measured by pseudopod formation in donor neutrophils exposed to shock plasma. Homogenates made of rat peritoneal organs do not significantly activate isolated naive neutrophils except for pancreatic homogenate, which contains factors that highly activate neutrophils in vitro. Because of the prevalence of proteases in this organ, the mechanism of neutrophil activation might be protease-coupled. The reported efficacy of protease inhibitors in shock and the deleterious systemic effects of circulating proteases as well as reported neutrophil activation by various proteases also point to a possible direct mechanism of neutrophil activation by pancreatic proteases.

To study this, pancreatic homogenate was assayed for its ability to activate isolated naive human neutrophils, in the presence and absence of various protease inhibitors. Rats randomly selected were weighted and anesthetized, and arterial and venous catheters were inserted. A laparotomy was made and the animals were exsanguinated. The pancreas immediately removed and put into 0.25 M sucrose solution and homogenized in 1:9 (w/v) Krebs-Henseleit solution. Aliquots of the homogenates were mixed with different protease inhibitors. Serine protease inhibitors proved effective at inhibiting the activation of human neutrophils incubated with rat pancreatic homogenate. The protease inhibitor with the greatest in vitro efficacy was Futhan (nafamostat mesilate), which abolished pancreatic homogenate-induced activation (p<0.001).

Because pancreatic homogenate activates endothelial cell cultures as well as naive neutrophils in vitro, it was tested to determine whether it activates other tissue homogenates. In vitro neutrophil activation by pancreatic homogenate was inhibited by the addition of serine protease inhibitors. Therefore, it was of interest whether the addition of pancreatic homogenate or exogenous serine proteases to other organs would result in neutrophil activation by non-pancreatic tissue. Organs from the rat in addition to pancreas were collected and homogenized, including spleen, proximal small intestine, heart, and liver. Aliquots of each sample were mixed with non-stimulatory volumes of either pancreatic homogenate or the serine proteases trypsin, chymotrypsin, or both. Suspensions were incubated for 2.5 hours at 38° C. and neutrophil actin polymerization (pseudopod formation) was measured. Results indicated a significant increase (p<0.01) in neutrophil activation by tissue homogenates incubated with either sub-stimulatory levels of pancreatic homogenate or serine proteases. Activation using organ homogenates form organs other than the pancreas was not elevated. These results indicate that tissue homogenates incubated with serine proteases contain factors that activate neutrophils in vitro. The pancrea may serve as an endogenous source for neutrophil activation.

7.1 Introduction

Splanchnic arterial occlusion (SAO) shock, in addition to other pathological etiologies such as hemorrhagic and endotoxic shock, releases circulating factors in the blood that have the ability to activate neutrophils in vitro (see Example 3). Tissue homogenates from the pancreas, but not from other organs studied, activate naive neutrophils as assayed by actin polymerization and superoxide formation tests (see Example 5). It is possible that the pancreas is an endogenous source for neutrophil activators in vivo as well. Such factors could be released in shock and other pathologic states as diverse as malnutrition and septicemia, and contribute to initial neutrophil activation and priming.

This study sought to identify substances produced in the pancreas that lead to neutrophil activation in vitro. The pancreas is an integral component of the splanchnic region, functioning as the principal player in two distinct digestive functions, endocrine and exocrine processes. These two functions use two different cell subsets in the pancreas. Beta cells of the Islands of Langerhans drive the endocrine function of the pancreas, contributing insulin directly to the blood stream in response to increases in blood-sugar levels. Other cells of the pancreas control the exocrine functions of the body. Acinar cells hold stores of largely inert pro-enzymes and other potentially catabolic substances which are released in response to digestive processes in the gut. Chief among these pancreatic substances are the proteolytic enzymes, which are released from a non-reactive zymogen form to an active enzyme by the actions of trypsin, itself cleaved from an inactive zymogen by the intestinal enzyme enteropeptidase (Table 7.1; adapted from Rinderknecht (1993) Chapter 12 in The Pancreas: Biology, Pathobiology, and Disease, Go et al., Ed., Raven Press, NY, pp. 219-251). Other pancreatic enzymes include lipase, carboxyl ester hydrolase, amylase, ribonuclease, and deoxyribonuclease 1.

TABLE 7.1
Schematic of pancreatic enzyme activation.

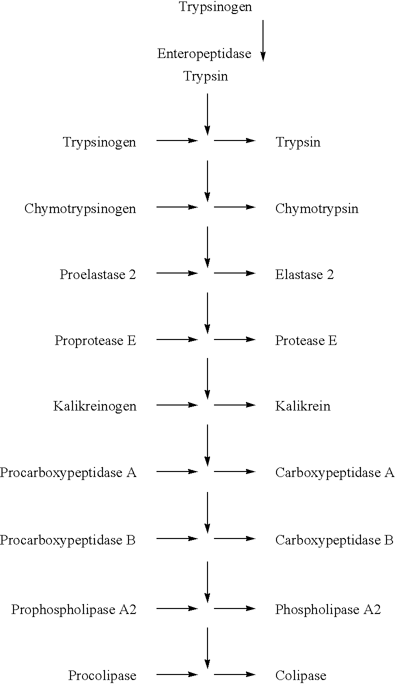

In the normal functioning pancreas, the activation of potentially autocatalytic enzymes is controlled by limiting the activation of trypsin until its delivery into the gut. In addition there co-exist with pancreatic enzymes anti-proteases such as pancreatic secretory trypsin inhibitor (PSTI) which is present in sufficient amounts to inactivate up to 20% of pancreatic trypsin. PSTI serves to limit any proteolytic activity that may occur as the result of inappropriate protease (auto)activation. Upon release in the plasma, pancreatic proteases can be inactivated by protease inhibitors such as α₁-proteinase inhibitor (α₁-antitrypsin), α₂-macroglobin, inter-α₁-trypsin inhibitor, and α₁-antichymotrypsin. Of these inhibitors α₁-proteinase inhibitor is by far the most concentrated, accounting for approximately 90% of the plasma protease screen. This antiprotease ‘screen’ is responsible for the inactivation of any proteases that arrive in the circulation. Despite the carefully controlled mechanisms of release for pancreatic digestive enzymes, these pancreatic proteases can be released under various pathological conditions and play important roles in various disease states, such as pancreatitis and shock. With depletion of the antiprotease screen, pancreatic and neutrophil proteases are free to circulate contribute to system-wide tissue destruction. Proteases from the pancreas are also thought to play a role in the initiation of endothelial free radical production by the transformation of membrane-bound xanthine dehydrogenase to xanthine oxidase. Upon reperfusion after ischemia, membrane-bound and circulating xanthine oxidase produce large quantities of oxygen free radicals, resulting in tissue damage and cytokine activation. Thus, inappropriate release of pancreatic enzymes may contribute to the initial neutrophil activation such as is seen in shock and pancreatitis.

The effect of protease inhibitors was measured in the study in an effort to determine whether in vitro neutrophil activating factors from the pancreas are protease in origin. In particular, it was of interest to determine the activity of the serine proteases of the pancreas, which have been implicated in the majority of pathologic actions attributed to proteases. So-called because of the serine moiety of the catalytic amino acid triad His-57, Asp-102, Ser-195 that makes up the active proteolytic site, proteases from this family are inhibited to varying degrees by serine protease inhibitors depending on the conformation of the particular pro-tease involved. A variety of different protease inhibitors were thus assayed for their ability to inhibit neutrophil actin polymerization (pseudo-pod formation) due to rat pancreatic homogenate application in vitro.

It was also of interest to determine whether inhibitory effects by proteases on neutrophil activation were homogenate or neutrophil-dependent. To answer this question, a series of experiments was conducted using neutrophils that had been incubated with a protease inhibitor and then washed of all unbound protease inhibitor. The inhibition seen in response to pancreas homogenate application to this “washed” sample was then compared to that of neutrophils incubated with the protease inhibitor that had not been washed away and was still present in the buffer. In this way it could be determined whether or not the protease inhibition was directed towards the pancreatic homogenate or the neutrophil population itself.

It was hypothesized that pancreatic proteases, in addition to degrading tissue and possibly forming neutrophil activating factors in the pancreas, play a similar role in other organs. Therefore, the ability of pancreatic homogenate and its principal proteases trypsin and chymotrypsin to induce other tissues to express neutrophil activating factors was studied.

7.2 Methods

Rat homogenate was collected as described in detail in Example 5. Briefly, male Wistar rats (250-350 gm) were housed in a controlled environment and maintained on a standard pellet diet for at least three days before initiation of experimental procedures. Animals were cannulated via the femoral arteries and vein under general anesthesia using pentobarbital (50 mg/kg i.m.). The rats were exsanguinated and the heart, liver, spleen, small intestine, and pancreas removed. The organs were immediately washed and cleaned in cold 0.25 M sucrose solution. Then, the cleaned organs were vigorously homogenized in Krebs-Henseleit solution (1:9 w/v). Aliquots were filtered by centrifugation at 500 G for 10 min. Pancreatic homogenates were incubated for 2.5 hours at 38° C., stirred frequently. Incubated pancreatic homogenate as well as controls were tested against naive human donor neutrophils for pseudopod formation as described in Example 2.

To determine whether or not neutrophil activation by pancreatic homogenate is protease dependent, the pseudopod formation test was repeated by preincubation of isolated neutrophils with various protease inhibitors (50 μl) for 10 minutes followed by addition of pancreas homogenate (50 μl) and further incubation for 10 minutes. The inhibitors used were Phenylmethylsulfonyl fluoride (PMSF) (1 mM), Complete™ with and without EDTA (1 tablet/20 ml), Benzamidine (1150 μM), Futhan (Nafamostat Mesilate) (0.1 mg/ml), and aprotinin (20 μM). All protease inhibitors were the gift of Dr. Tony E. Hugli of The Scripps Clinic and Research Institute, with the exception of Complete™, an all-purpose protease inhibitor purchased from Boehringer Mannheim, Indianapolis, Ind. One of the components of Complete™ is EDTA (ethylenediaminetetra-acetic acid), a standard anti-coagulant and divalent cation chelator. The calcium scavenging effect of EDTA also inhibits neutrophil response to stimuli and thus the inhibitory effect of Complete ™ was assayed with and without the addition of 70 μM MgCl₂ to bind to soluble EDTA, as per Company instructions.

To determine whether the protease inhibitors were acting on the pancreatic homogenate or the neutrophils themselves, a new set of neutrophil pseudopod experiments was carried out. Three groups were used: A Control group of isolated human neutrophils (100 μl of 10⁶ cells/ml) that had been washed two times in D-PBS and incubated with 50 μl of pancreatic homogenate for 10 minutes; a Wash group of isolated human neutrophils (100 μl of 10⁶ cells/ml) incubated for 10 minutes with 50 μl Futhan (0.1 mg/ml), washed two times in D-PBS to remove unbound Futhan and then incubated with 50 μl of pancreatic homogenate for 10 minutes; and an Inhibitor group of isolated human neutrophils (100 μl of 10⁶ cells/ml) that had been washed two times in D-PBS, incubated for 10 minutes with combined 50 μl Futhan (0.1 mg/ml) and 50 μl of pancreatic homogenate, which had previously been incubating together for 10 minutes. Appropriate controls were recorded as well as a positive control (10⁻⁷ M fMLP) to determine the washed cells' ability to respond to stimuli. Formyl-methionyl-leucyl-phenylalanine (fMLP) was obtained from Sigma Chemical Company, St. Louis, Mo.

To determine the in vitro effect of the addition of serine proteases to other organ homogenates, homogenates of spleen, heart, small intestine, and liver were divided into aliquots and incubated with either sub-activating concentrations of pancreatic homogenate (100 μl filtered pancreatic homogenate/3 ml organ homogenate) as verified by previous assay, trypsin (2600 U/ml homogenate), chymotrypsin (104 U/ml homogenate), trypsin (1300 U/ml)+chymotrypsin (52 U/ml), or comparable volumes of a control solution (Krebs-Henseleit solution). These previously non-incubated samples were then incubated in the usual manner at 38° C. for 2.5 hours, stirred occasionally. Once prepared, samples were measured within 24 hours as activation in test homogenates decay with a half-life on the order of approximately 24-48 hours.

In separate sets of experiments, trypsin and chymotrypsin, as well as their precursors trypsinogen and chymotrypsinogen were tested by pseudopod formation for their ability to activate naive neutrophils. In addition, trypsinogen and chymotrypsinogen activated by trypsin were also tested for their ability to activate quiescent neutrophils. Trypsin (Type 11-S from porcine pancreas) (α-chymotrypsin (Type II from bovine pancreas), trypsinogen (from bovine pancreas), and α-chymotrypsinogen (Type II from bovine pancreas) were obtained from Sigma Chemical Company, St. Louis, Mo.

Results were expressed as Mean±SD for all samples. The paired Student's t-test was used for tests measuring pseudopod formation of samples with and without addition of activators and a two-tailed unpaired Student's t-test was used for all other comparisons. Differences with P<0.05 were considered significant.

7.3 Results

The application of protease inhibitors on neutrophil pseudopod formation by rat pancreatic homogenate resulted in a decrease in neutrophil activation that varied depending on protease inhibitor used. Benzamidine (n=7) and aprotinin (n=9) were the least effective, but still resulted in significant inhibition of neutrophil activation by pancreatic homogenate from 85.1±11.5% to 41.5±24.6% and 51±17.2% respectively (P<0.05). Complete™ (n=9) was effective at blunting the response to pancreatic homogenate (46.8±13.4%) (P<0.005) and there was substantial inhibition of neutrophil pseudopod activation by PMSF (n=5) (40.8±12.9%) and especially Futhan (n=15) (30.3±11.8%) (P<0.001). In subsequent experiments, Futhan was used exclusively (except where noted) as an inhibitor of neutrophil activation by pancreatic homogenate. Inhibition of neutrophil activation by protease inhibitors as assayed by the pseudopod formation test was dose-dependent. Application of protease inhibitors also does not appear to be pro-inflammatory in the concentrations used.

Experiments to determine the mechanism by which the protease inhibitors inhibit neutrophil activation by pancreatic homogenate were largely indeterminate. Isolated human neutrophils that were washed twice with D-PBS were not substantially activated (9% activation (n=2)) but retained their ability to be activated by fMLP (74% activation (n=2)) as well as by pancreatic homogenate in the Control group (69.8±21% activation (n=10)). There was slight inhibition of this activation by the protease inhibitor Futhan (0.1 mg/ml), with neutrophils that had been pre-incubated with Futhan and rinsed two times to remove excess Futhan (Wash group) (53.5±31% activation (n=8)) and those neutrophils that were incubated with Futhan concurrent with pancreatic homogenate after rinsing (Inhibitor group) (51.5±34% activation (n=8)) (Pseudopod test comparison of pancreatic homogenate applied to naive washed (2×) neutrophils (Control Group [n=10]), with Futhan-treated and washed (2×) neutrophils (Wash Group [n=8]) and naive washed (2×) neutrophils incubated with Futhan and then pancreatic homogenate (Inhibitor Group [n=8]). Untreated naive cells (washed 2×) and fMLP-treated (10-7 M) neutrophils are negative and positive controls, respectively. No significant difference between Wash and Inhibitor Groups or between Control Group and Wash and Inhibitor Groups (P=0.1) were observed).

Incubation of pancreatic homogenate in sub-activating concentrations with other organ homogenate for 2.5 hours at 38° C. resulted in a significant increase in percent pseudopod formation in liver homogenate (n=6) (P<0.05) from 8.3±5.2% to 38±26.5% and intestine (n=6) (P<0.001) homogenate from 27.7±19.9%7 to 78±12.1%, as well as non-significant increases in pancreas-incubated heart from 15±14.1% to 35.3±37.2% (n=6) and spleen homogenates from 33.1±15.8% to 43.2±23.2% (n=6) compared to non-pancreatic incubated homogenates (n=5). There was also a significant difference in pseudopod activation between pancreatic-incubated controls and pancreatic-incubated spleen homogenate (P<0.01) and intestine homogenate (P<0.001) as well as nonsignificant increases in percent pseudopod formation in pancreatic-incubated heart and liver homogenates. There was borderline significance between control activation (10.5±2.5) and pancreas-incubated controls (18.1±7.5%) (P=0.05) (Percentage of neutrophils displaying pseudopods after mixing with either organ homogenate (n=5) or organ homogenate (n=6) incubated with filtered pancreatic homogenate (100 μl pancreatic homogenate/3 ml organ homogenate).

Incubation of organ homogenate with the serine proteases trypsin+chymotrypsin for 2.5 hours at 38° C. resulted in a significant increase in percent pseudopod formation in all tissue homogenates (n=6) (P<0.001) compared to non-protease incubated homogenates (n=5). Activation in liver homogenate increased from 8.3±5.2% to 74.4±24.4%, spleen activation increased from 33.1±15.8% to 72.75±17.4%, heart activation increased from 15±14.1% to 78.9±13.3%, and intestine activation increased from 27.7±19.9% to 65.7±8.9%. There was also a significant difference in percent pseudopod formation between protease-incubated controls (n=4) and all tissue homogenates studied (P<0.01 for spleen homogenate, all other tissues P<0.001). There was no significant difference between control activation (10.5±2.5%) and protease-incubated controls (9.6±5%) (Percentage of neutrophils displaying pseudopods after mixing with either organ homogenate (n=5) or organ homogenate (n=6) incubated with serine proteases chymotrypsin (52 U/ml homogenate) and trypsin (1300 U/ml homogenate)).

Incubation of organ homogenate with the serine protease trypsin alone for 2.5 hours at 38° C. resulted in a significant increase in percent pseudopod formation in all tissue homogenates (n=4) (P<0.001) compared to non-trypsin incubated homogenates (n=5). Activation of liver homogenate increased from 8.3±5.2% to 84.5±9.47%, spleen activation increased from 33.1±15.8% to 67±12.2%, heart activation increased from 15±14.1% to 87.75±7.1%, and intestine activation increased from 27.7±19.9% to 69.2±12.8%. There was also a significant difference in percent pseudopod formation between trypsin-incubated controls (n=2) and all tissue homogenates studied (P<0.01 for intestine homogenate, all other tissues P<0.001). There was no significant difference between control activation (10.5±2.5%) and trypsin-incubated controls (8.5±0.8%) (Percentage of neutrophils displaying pseudopods after mixing with either organ homogenate (n=5), or organ homogenate (n=4) incubated with trypsin (2600 U/ml organ homogenate)).

Singles samples of organ homogenate incubated with the serine protease chymotrypsin for 2.5 hours at 38° C. resulted in an increase in percent pseudopod formation in all tissue homogenates. Increases in chymotrypsin-induced percent pseudopod formation were most pronounced in liver homogenate and heart homogenate which rose from 7.4±5.1% to 99% and 15±13% to 95%, respectively. Spleen activation increased from 29.1±17.9% to 47%, and intestine activation increased from 24.3±29.3% to 80%. The chymotrypsin-incubated control sample activation increased from 8.1±5.1% to 42%, a much greater increase than normally seen with this agent (author's notes) (Percentage of neutrophils displaying pseudopods after mixing with either organ homogenate (n=5), or organ homogenate (n=1) incubated with chymotrypsin (104 U/ml organ homogenate)).

7.4 Discussion

These studies demonstrate that neutrophil activation by pancreatic homogenate, as assessed by actin polymerization, is inhibited by the application of serine protease inhibitors in vitro. Of the protease inhibitors studied, Futhan performs the most effective in vitro inhibition of pancreatic neutrophil activating factors. Futhan, FUT-175, or nafamostat mesilate (6-amidino-2-naphthyl p-guanidinobenzoate dimethanesulfonate) as the compound is known, is a low-molecular weight serine protease inhibitor produced by Torii & Company, Ltd (Tokyo, Japan). It is a broad spectrum inhibitor for trypsin, chymotrypsin, elastase, kallikrein, plasmin, thrombin, enterokinase, Clr, and Cl esterases as well as phospholipase A₂. It is used clinically in Japan principally for coagulation disorders such as disseminated intravascular coagulation (DIC) as well as acute pancreatitis.

In vitro it inhibited zymosan-induced chemiluminescence activation by neutrophils, but does not possess any free radical scavenging effects, a criticism about specificity that has been leveled at other serine protease inhibitors. Futhan displays a dose-dependent inhibition of neutrophil actin polymerization by incubated rat pancreatic homogenate.

The finding that neutrophil activation by rat pancreatic homogenate can be inhibited by Futhan points to the involvement of serine proteases in the activation process, either as a soluble activator of neutrophils or as an intrinsic component of the neutrophil surface receptor response. It also indicates that Futhan can be used as an agent for activation lowering therepy.

Because incubated pancreatic homogenate contains appreciable levels of proteases, these enzymes were candidates for large-molecular weight (20-40 kD) neutrophil activating factors. Although it has been shown that serine proteases such as trypsin and chymotrypsin can amplify neutrophil activation to stimuli such as fMLP and phorbol myristate acetate (PMA) (amplification strength: cathepsin G>chymotrypsin>elastase>trypsin), and they have been reported to cause apoptosis in higher concentrations, these proteases have not been reported to activate neutrophils.

This has been demonstrated by studies herein in which the application of trypsin and chymotrypsin did not result in neutrophil activation either by pseudopod formation or chemiluminescence (see Example 6.4). Rather, (pancreatic homogenate induced) neutrophil activation appears to involve a chymotrypsin-like protease on the surface of the neutrophil, which can be inhibited by the application of protease inhibitors. This receptor molecule is thought to be CD43, which appears to work as a “functional barrier” to neutrophil activation, as assayed by opsonized zymosan and PMA. Proteolytic (chymotrypsin-like) cleavage of CD43 may be a required event for neutrophil activation.

It was ascertained whether the inhibition of neutrophil activation by pancreatic homogenate in vitro by serine protease inhibitors was dependent on protease inhibitor inactivation of pancreatic homogenate factors or the neutrophils. Neutrophils that had been washed of the free (circulating) protease inhibitor Futhan displayed comparable inhibition of pancreatic homogenate-induced activation to neutrophils which were incubated in Futhan+pancreatic homogenate. Because the levels of inhibition seen in this experiment were less than in other Futhan inhibition studies (51-53% activation compared to a typical inhibition of neutrophil activation to approximately 30%) it is not possible to draw a definitive conclusion from these experiments. Comparable neutrophil inhibition was achieved whether the protease inhibitor was applied and subsequently the non-bound inhibitor washed out or the protease inhibitor was applied directly to the neutrophils with the homogenate. Therefore, it appears that the inhibitory actions of Futhan are directed predominately at the neutrophil membrane chymotrypsin-like receptor and not at factors in the homogenate itself. Lack of complete inhibition in the Wash group can be attributable to a possible lack of total receptor binding after washing, allowing activating factors to upregulate the neutrophils. In the non-washed or Inhibitor group, Futhan binding of free pancreatic proteases possibly diminished effective Futhan concentration, inhibiting the protease inhibitor's ability to compete effectively for binding sites. From these results it can be theorized that pancreatic proteases, while probably playing a synergistic role in ‘priming’ neutrophils for activation, are not endogenous pancreatic neutrophil activator.

This finding does not, however, address the role of pancreatic proteases in the synthesis and release of pancreatic neutrophil activators. It is known that pancreatic enzymes are intimately involved in the auto-destruction of pancreatic tissue and the release of toxic factors. In addition to trypsin and chymotrypsin, other enzymes thought to be of importance in the pathologic pancreas include lipase and elastase, which are implicated in the autodigestive process of the pancreas.

Incubation of previously non-reactive tissues with sub-activating concentrations of pancreatic homogenate resulted in significant levels of neutrophil activating factors in all organs tested: the spleen, heart, liver, and small intestine. This result could be repeated by incubating the tissues with either trypsin, chymotrypsin, or both. This finding points to a defined in vivo role for the pancreas in neutrophil activation in shock and other deleterious conditions as circulating pancreatic enzymes are routinely measured in diseased states. The identity of the protease-released neutrophil activating factors is not yet clear. One possible component factor are the platelet activating factor (PAF)-like substances, which have been shown to be synthesized and released from endothelium in response to simulation by proteases such as chymotrypsin and thrombin. It is possible that release of pancreatic proteases into the circulation results in the formation and release of neutrophil activating factors by organs other than the pancreas, especially in the event of a compromised antiprotease plasma screen.

It is possible that neutrophil activating factors released by protease incubation in previously non-activated tissues are identical to those released by the pancreas itself. There are several lines of evidence that point against this. First, although pancreatic homogenate increases in potency to some degree after incubation at 38° C. for 2.5 hours to maximize proteolytic processes, it possesses neutrophil stimulating activity even without incubation, implying that protease activation is not necessary for expression of this factor (see Example 5). Second, the half-life time courses of in vitro neutrophil activation potency differ greatly between pancreatic homogenate and protease-incubated tissues. The neutrophil activating component of pancreatic homogenate is stable for extended periods of time when stored at 4° C. Protease-incubated tissues, on the other hand, decay in potency almost immediately and return to control levels within days. Third, incubation of tissues at 38° C. for 2.5 hours prior to repeated incubation at 38° C. for 2.5 hours in the presence of proteases appears to inhibit the appearance of neutrophil activating factors (author's notes) while incubation of pancreatic homogenate for extended periods of time (4+ hours) at 38° C. appear to have no effect on potency.

Neutrophil activation in response to incubation with rat pancreatic homogenate results in very high levels of neutrophil activation in vitro. This activation can be inhibited by the application of exogenous serine protease inhibitors, of which Futhan performs the most effectively. The mechanism of this inhibition is unknown, but is believed to be due, in part to the inhibitory actions of protease inhibitor binding to CD43 on the neutrophil membrane surface. Application of sub-stimulatory levels of pancreatic homogenate or the serine proteases trypsin or chymotrypsin to otherwise non-stimulatory tissue results in the release of neutrophil activating factors by these tissues. The identity of these stimulatory factors is not clear, but they appear to be different in nature from those released by the pancreas. The pancreas, through the release of endogenous neutrophil activating factors as well as proteases that upregulate neutrophil activating factors in other tissues, may be a principal source of neutrophil activating substances in the body.

7.5 Pancreatic Activating Factors and the Neutrophil Response to Shear Stress In Vitro

7.5.A Introduction

The aim of this study was to provide further in vitro evidence of the excitatory effect of filtered pancreatic homogenate on neutrophils by observing this factor's inhibitory effect on neutrophil response to fluid shear-stress. Leukocytes migrate from a hemopoietic pool across marrow endothelium into the circulation and, under inflammatory circumstances, from the circulation across the endothelium to sites of inflammation. These migrations require adhesion of the leukocyte to the endothelium and pseudopod formation. Pseudopods (also known as microvilli lamellipods) occur as protrusions on the cell surface and can be encountered on endothelial cells as well as on leukocytes. These protrusions are strongly related to the formation (polymerization) of the F-actin network. Pseudopods are stiffer than the main cell body, and therefore circulating activated neutrophils have greater difficulty in passing through capillaries.

It has been previously demonstrated that fluid shear-stress (˜I dyn/cm²) causes naive human neutrophils to retract their pseudopods within seconds. The mechanism involved in this response in not clear, but it is believed to involved Ca⁺⁺ and K⁺ flux as well as cyclic GMP.

To better define the actions of the new leukocyte activating factor originating in the pancreas, its effects on human neutrophils in terms of shear-stress response was investigated.

7.5.b Methods

In these experiments the shear-stress was kept sufficiently low (˜1 dyn/cm2) to avoid significant viscoelastic cell deformation. Fresh leukocytes from pin-pricked blood of healthy volunteers were collected in Vacutainer tubes (sodium heparin pre-treated, Becton Dickinson, Franklin Lakes, N.J.). The red blood cells were allowed to sediment at room temperature for about 30 minutes. The supernatant mixture of 1 part platelets and leukocytes was resuspended in 20 parts Plasma Lyte (Baxter Healthcare Corp., Deerfield, Ill.). Neutrophils, monocytes and lymphocytes were identified by their morphology (125× magnification). All experiments were performed within four hours following blood collection.

Rat and pig pancreatic homogenate was collected as described in Example 7.2 and in further detail in Example 5. Low molecular weight aliquots were filtered as previously described with a 3 kD cut-off.

100 μl of the cell preparation was deposited into a small chamber with a transparent bottom on an inverted microscope (Leitz Diavert, Germany) with a 50× objective. The microscope light source had a heat filter and all experiments were carried out at room temperature. The microscope eyepiece was connected to a closed circuit TV system, with a black and white coupled charge device camera (Model JE2362, Javelin, Japan) with a 25× objective, analog background subtraction (Model LKH 9000, L. K. Hawke Inc., Research Triangle Park, N.C.), video timer (Model G-77, Odetics, Anaheim, Calif.), VHS video cassette recorder (Model AG 1270, Panasonic, Japan) and monitor (Model VM 4512, Sanyo, Japan) for playback analysis.

Micropipettes were fabricated using a micropipette puller (David Koph Instruments) (internal diameter ranging from 1-3 μm). The micropipettes were connected to a reservoir with hydrostatic pressure adjustment.

Adherent leukocytes, which were spread on the glass surface, were identified and a single micropipette was positioned above the cell so that a jet of fluid could be applied over its surface. The micropipette was inclined at approximately 30° to the surface and the tip of the pipette is 5 μm from the center of the cell surface. Numerical computation (see, Trapali et al. (1996) Life Sci 59:849-857) gave a centerline velocity of the fluid jet out of the pipette tip of 0.74 m/s and a shear-stress over the cell surface ranging from 0.02 dyn/cm² to 0.4 dyn/cm².

90 μl of the cell suspension was deposited into the small chamber. 10 μl of pancreatic homogenate were then added to the cell suspension. In experiments with Futhan 10 μl of pancreatic homogenate and 10 μl of Futhan at various concentrations were added to 80 μl of the cell suspension. In each observation one isolated activated neutrophil was selected and filmed for 7 minutes. Shear stress was applied after 2 minutes and for a period of 3 minutes.

7.5.c Results

Filmed neutrophils presented various responses to shear-stress. In order to quantify the results neutrophil response was divided into three categories:

Complete response: the neutrophil retracts all its pseudopods during application of shear-stress and assumes a circular shape. It projects them again after cessation of flow

Partial response: the neutrophil retracts some of its pseudopods but not all of them or projects new pseudopods

No response: the neutrophil does not retract any of its pseudopods

Percentages of the 3 different types of responses for different groups of cells in incubated with pancreatic homogenate were measured. Rat 1 and Rat 2 were treated with filtered pancreatic homogenate from 2 different rats. Pig 1 Low MW and Pig 2 Low MW were treated with the low molecular weight fraction (<3 kD) of pancreatic homogenates from 2 different pigs. Pig 2 (whole) was treated with the whole pancreatic homogenate from the same pig as Pig 2 Low MW.

All control cells completely responded to shear-stress whereas less than 20% of the cells treated with the pancreatic homogenates completely responded to shear-stress. 20-50% of the treated cells presented no response to shear-stress. Porcine pancreatic homogenate appeared to display a stronger effect than the rat pancreatic homogenate but this observation needs to be confirmed by further experiments. No significant difference was found between the whole pancreatic homogenate and its low molecular weight fraction in the inhibition of the shear-stress response in the limited sample studied (13 cells were observed). To investigate the mechanisms by which pancreatic activating factors act on neutrophils, mitigation of its effects by the serine protease inhibitor Futhan were measured. 10 μl of Futhan (0.05 and 0.13 mg/ml dissolved in 5% glucose) were added to 80 μl of cell suspension and 10 μl of pig pancreatic homogenate. Results indicated that Futhan in these concentrations has a slightly pro-inflammatory effect on neutrophil activation as assayed by this test; no Futhan-treated neutrophil exhibited a complete response to shear stress.

7.5.d Discussion

Pancreatic homogenate from either rats or pigs partially inhibited the normal response to shear-stress of naive human neutrophils in vitro. This indicates the presence of one of more neutrophils activating factors present in the low molecular weight fraction (<3 kD) as well as possibly also at higher molecular weights. Contrary to pseudopod formation results in vitro, Futhan did not appear to down-regulate neutrophil activation by pancreatic homogenate as measured by the response to shear-stress. The reasons for this lack of inhibition are unclear but may have to do with the low pH of soluble Futhan, (see, Example 8).

### EXAMPLE 8

### Beneficial Effects of a Serine Protease Inhibitor in Splanchnic Arterial Occlusion (SAO) Shock

Summary

Plasma factors from splanchnic arterial occlusion (SAO) shock, like hemorrhagic and endotoxic shock, result in upregulated levels of leukocvte activation, as measured by nitroblue tetrazollum (NBT) and pseudopod activation tests in the rat. Also, homogenate from the pancreas, but not from other tissues tested will activate naive neutrophils by these same tests. This activation was inhibited in part by the application of serine protease inhibitors, in particular by Futhan (nafamostat mesilate).

It was then determined that this activation could be inhibited in vivo. Rats randomly selected were weighed and anesthetized, and arterial and venous catheters were inserted which were used for blood pressure measurements and anesthesia, respectively. In addition, a second venous catheter was inserted and connected to an infusion pump which injected Futhan or a comparable volume of saline at the rate of 3.3 mg/kg body wt per hour. After a one hour pre-treatment period, a laparotomy was made and the superior mesenteric artery and celiac artery were clamped for a period of 90 minutes, at which time the clamps were removed. Animals were observed for survival for 60 minutes after reperfusion or until such time as the mean arterial pressure fell below 30 mmHg. At the termination of the experiments arterial blood was drawn for determination of plasma peroxide concentration using a peroxide electrode measurement technique.

Results indicate a significant difference in blood pressure after reperfusion between SAO shock and Futhan-treated SAO shock animals (P<0.005 for all time points greater than 90 minutes), as well as a significant increase in survival of Futhan-treated animals compared to non-treated controls (P<0.001). Peroxide levels in Futhan-treated SAO shock plasma were also significantly less than those in non-treated SAO shock animals (P<0.05), although both values were significantly greater than initial plasma peroxide levels (P<0.001). These results indicate that SAO shock can be mitigated by pre-treatment with the serine protease inhibitor Futhan and that some of this protection may derive from the ability of the protease inhibitor to limit the concentration of activators in the circulation during shock as measured by plasma peroxide formation.

Factors in pancreas homogenate activated naive neutrophils in vitro. Because of the extensive involvement of neutrophils in SAO shock it was hypothesized that activators produced by the pancreas are sufficient in themselves to stimulate neutrophils in vivo and contribute to the shock condition. The serine protease inhibitor Futhan inhibited the upregulation of naive neutrophils exposed to incubated pancreatic homogenate as well as mitigate SAO shock in vivo as reported in these studies.

A bolus injection of incubated pancreatic homogenate was tested for its ability to lead to circulatory shock in the rat. The ability of Futhan pretreatment to mitigate shock induced in this manner was also tested. A mock SAO shock protocol was repeated as previously described, with either 60 min Futhan or saline pretreatment and a 2 ml bolus injection of either pancreatic homogenate or low-molecular weight pancreatic homogenate injected in lieu of arterial clamping. Injection of whole pancreatic homogenate proved immediately fatal to saline-treated controls while Futhan-treated rats recovered after a brief hypotension (P<0.001 blood pressure between groups after injection). Repeated experiments with 3 ml of low-molecular weight pancreatic homogenate resulted in transient decreases in blood pressure in response to homogenate (P<0.001 compared to initial pressure) from which the animals subsequently recovered.

In order to study the physiological actions of pancreatic homogenate upon the microcirculation in situ, a fluorescent intra-vital preparation was made of the rat mesentery, which was superfused with pancreatic homogenate or control buffer. Superfusion of pancreatic homogenate resulted in a marked increase in DCFH neutrophil fluorescence, an index of hydrogen peroxide formation. Propidium iodide fluorescence, used index of hydrogen peroxi for the measurement of cell death, increased but was not significantly different from increases in control animals. Superfusion of whole pancreatic homogenate also resulted in significantly increased neutrophil adhesion and microcirculatory vaso-constriction. These results suggest an in vivo role for bioactive factors released from the pancreas in shock and in other pathologic events.

8.1 Introduction

Neutrophil activation was produced in vitro by the application of homogenate from the pancreas but not by application of homogenate from other organs tested. This activation was inhibited by different serine protease inhibitors. Futhan was found to be the most effective. Splanchnic arterial occlusion (SAO) shock is a shock model that targets the splanchnic region, in particular the pancreas and leads to systemic upregulation of neutrophils.

Thus, systemic in vivo neutrophil activation may arise in this model due in part to inappropriate release of neutrophil activating factors from the pancreas. This study sought to determine whether the deleterious effects of SAO shock could be mitigated by the interventions of Futhan in vivo.

The release of pancreatic constituents may be an important event in neutrophil activation and the pathogenesis of shock. Because of the presence of neutrophil activating factors in the pancreas as well as high concentrations of serine proteases, which create neutrophil activating factors (Example 7), it was hypothesized that the pancreas contains sufficient concentrations of activators and toxins to initiate acute shock without participation of other stimuli. SAO shock experiments were repeated as reported in Example 7 with a bolus injection of pancreatic homogenate simulating the unclamping of the splanchnic arteries and release of pancreatic contents as is seen in SAO shock. Because of the serine protease Futhan's ability to mitigate neutrophil activation in vitro, it was also hypothesized that Futhan pre-treatment would be beneficial in mitigating the effects of a bolus injection of pancreatic homogenate.

In addition, because of the findings of a low-molecular weight neutrophil activator in pancreatic homogenate, it was of interest to determine whether this factor or other colocalized low-molecular weight species were sufficient to induce shock in rats. Simulated SAO shock was repeated with low-molecular weight pancreatic homogenate bolus injections mimicking the release of arterial clamps.

After studying the effects of pancreatic homogenate on neutrophil function in vitro and whole animal response In vivo, it was of interest to determine physiological mechanisms of pancreatic homogenate that lead to shock in vivo. To understand this in situ, an intravital fluorescent microscopy preparation was studied of the rat mesentery and the effect of exogenous filtered pancreatic homogenate was observed.

8.2 Methods

8.2.a Methods: SAO Shock Futhan Pretreatment Experiments

Methods are as reported in Example 4 with modifications. Male Wistar rats (250-320 g) were randomly divided into SAO shock (n=7) and SAO shock sham groups (n=6). Animals were cannulated via the femoral arteries and vein under general anesthesia using pentobarbital (50 mg/kg i.m.). No heparin was injected other than that needed to ensure open catheter lines (10U/ml Plasma-Lyte). One femoral artery was connected up to monitor continuous mean arterial pressure (MAP), while the other was used for the collection of blood samples. A second venous catheter was inserted and connected to an infusion pump, which injected Futhan or a comparable volume of saline at the rate of 3.3 mg/kg body wt per hour. MAP and heart rate were recorded. Preliminary experiments used mini bolus injections of Futhan at concentrations ranging from 1-20 mg/kg body wt per hour in lieu of an infusion pump. After a one hour pretreatment period, a laparotomy was made and the superior mesenteric artery and celiac artery were clamped for a period of 90 minutes, at which time the clamps were removed. Animals were observed for survival for 60 minutes after reperfusion or until such time as the mean arterial pressure fell below 30 mmHg.

At the termination of the experiments arterial blood was drawn for determination of plasma peroxide concentration using the peroxide electrode measurement technique as described in Example 2.

8.2.b Methods: Pancreatic Homogenate Injection Experiments

The SAO shock experiments were repeated with the bolus injection of pancreatic homogenate in an effort to simulate the “unclamping” of the arteries and liberation of pancreatic components into the circulation instead of the actual ischemia/reperfusion of the splanchnic region. The low-molecular weight (2 ml) as well whole homogenate (2 ml) were tested for their ability to induce hypotension and cause circulatory shock. The ability of Futhan to counter the effects of the injection of pancreatic homogenate was also tested as in the SAO shock protocol. At the termination of these experiments arterial blood was drawn for the determination of plasma peroxide concentration using the peroxide electrode measurement technique.

Futhan, or nafamostat mesilate (6-amidino-2-naphthyl p-guanidino-benzoate dimethanesulfonate) as the compound is known, is low-molecular weight serine protease inhibitor produced by Torii & Company, Ltd (Tokyo, Japan) and was the kind gift of Dr. Nobuhiro Ohmura, Jichi Medical School, Saitama, Japan (see Example 7).

8.2.a Methods: Intravital Fluorescent Microscopy Experiments

The intravital fluorescent microscopy of the rat mesentery preparation has been previously described in Example 3. The superfusate reservoir is under a vacuum and connected directly to a perfusion pump which can be adjusted to supply a variable flow-rate stream over the mesentery. It is recirculated after collection from a partioned stage to the reservoir. Alternatively, a bypass circuit permits circulation of liquid without superfusion to the stage.

The protocol was modified by the substitution of the Krebs-Henseleit superfusate buffer with Plasma-Lyte (Upjohn Comp., Kalamazoo, Mich.), a physiological buffer that does not require continuous nitrogen degassing. To enable mesenteric superfusion with limited volumes of purified pancreatic homogenate, a recirculating drip system was devised to ensure continuous superfusion of pancreatic homogenate. Plasma-Lyte was held in a reservoir (60 ml) under negative pressure which was connected by polyurethane tubing to an infusion machine, which pumped superfusate through a three-way stopcock to either recirculate the fluid or superfuse the preparation. After superfusion of the mesenteric preparation the superfusate drained through specially drilled holes in the animal stage and the fluid returned to the reservoir. The animal stage was segmented to avoid contamination of superfusate with animal fluids. The animal was raised from the floor of the stage, and animal effluent was transported under negative pressure to a waste container. Rats with abdominal blood or bleeding from laparotomy were not used.

After a 10 minute stabilization period, the mesenteric preparation was superfused with propidium iodide (PI) (IμM) (Sigma Chemical Co., St. Louis, Mo.) and dichlorofluorescein diacetate (DCFH-DA) (10 μM) (Molecular Probes, Eugene, Oreg.) were added to the reservoir and background autofluorescence was recorded in selected tissue areas. A first reading was then taken of bright-field (40× water-immersion, Leitz) and fluorescent images of selected venules and arterioles (20 μm-100 μm). 5-6 observation fields were selected at random and bright-field, PI, and DCFH readings were recorded every 20 minutes via a CCD camera connected to a video cassette recorder. Images were recorded for later analysis. Fluorescence light excitation exposure time was minimized to avoid photobleaching.

At 10 min after the first reading 3 ml of filtered pancreatic homogenate were added to the reservoir. Filtering homogenate by centrifugation at 500 G and subsequent filtering over filter paper (0.78 μm vacuum filter (Millipore Filter Co., Beverly, Mass.)) was necessary to prevent any opacity that might arise from the presence of large solutes. After 10 minutes, the second set of readings was made and readings were continued every 20 minutes for 120 minutes, at which time the experiments were terminated.

Video tapes were replayed for analysis of cell death, as determined by PI and hydrogen peroxide production, as measured by DCFH. For analysis venules were restricted to 20-80 μm in diameter. The number of PI-positive cells was calculated at initial time points in 5-6 arbitrarily defined regions of the mesentery, taken every 20 minutes. The entire field-of-view was used for this purpose, approximately, 300 μm×300 μm. The number of dead cells was compared at different time periods throughout the experiment DCFH fluorescence was recorded along the entire length of the venule in question and compared with background fluorescence in the interstitium (NIH image and Adobe Photoshop software packages). DCFH fluorescence was compared at 20 minute periods throughout the experiment. In addition, leukocyte sticking and vessel diameter was recorded throughout the experiment. Leukocytes were counted as mean number of stationary cell throughout a 30 second period. Vessel diameter was measured at a defined position on each recorded vessel, arbitrarily chosen, and expressed as normalized mean to account for differing vessel diameters. Length was compared to a standard and calculated using NIH Image software package.

Statistical analysis of the significance in the differences in DCFH fluorescence, PI cell death, vessel diameter and leukocyte adherence were determined by Student's t-test and expressed as mean values with standard deviation.

8.3 Results

SAO shock resulted in uniform hypotension and death upon release of clamps of the superior mesenteric and celiac arteries after 90 minutes as was reported in Example 4. Initial attempts at preventing the fall in blood pressure and subsequent death after reperfusion with small bolus injections (0.05 ml) Futhan were unsuccessful. Futhan injected at concentrations of 1 (n=3), 3.3 (n=3), 10 (n=3), and 20 (n=2) mg Futhan/kg body wt every 5 minutes with pretreatment times of either 60 minutes before shock (1, 10 and 20 mg/kg) or 30 minutes before occlusion (3.3 mg/kg) were uniformly unable to restore MAP or increase survival times. Likewise, in these initial experiments Futhan proved ineffective at mitigating the increase in plasma peroxide formation seen after SAO shock. There are no significant differences between controls and Futhan-treated groups (P<0.005 after SAO shock (n=9) compared to before SAO shock (n=7) in both groups).

The reasons for the ineffectiveness of Futhan in ameliorating SAO shock in initial experiments appeared to be due to the pH of Futhan in solution, which is approximately 3.5 at the concentration of I mg/ml in 5% glucose-deionized (DI) water as per the manufacturer's instructions. Under the established protocol, approximately 2 ml of this solution were injected at the rate of 0.05 ml every 5 minutes into an approximate rat blood volume of 20 ml. In sham-shocked animals (not reported here) the buffering capacity of blood is able to absorb any perturbations in pH.

In shocked animals, however, the pH buffering capacity, especially of the pancreas and small intestine, is severely compromised. Thus, the injection of Futhan would contribute to irreversible acidosis. Injection of Futhan caused transient hypotension when injected in 0.05 ml doses. Although the animals recovered from these transient depressions of MAP, Futhan injected animals typically expressed a lower MAP than the saline-injected group. This difference was not significant. Futhan was found to be insoluble in alcohol, rat serum, DMSO, and balanced Tris (Trizma) buffer at physiologic pH.

The difficulties of Futhan's low pH and hypotensive effects were mitigated by the continuous injection of Futhan (Futhan was infused at a concentration of 3.3 mg/kg body wt/hour via infusion pump) i.v. via an infusion pump which infused a high concentration of Futhan (20 mg/ml) at very low flow rates to achieve an effective injection concentration of 3.3 mg/kg body wt/hour. In this way, the injected volume was reduced to less than 1 ml (approximately 0.8 ml) and the possibility of acidosis was minimized. At this concentration (20 mg/ml) care must be taken to ensure that Futhan does not crystallize in the catheter.

Futhan pretreatment using this modified protocol for 60 minutes resulted in significant reduction in the fall of blood pressure at all time points after the removal of arterial clamps (P<0.005). Survival time was also improved as Futhan prevented mortality in Futhan-pretreated animals after SAO shock at the end of 30 minutes reperfusion time. None of the saline-treated control animals survived to this time point.

In addition to improvement of blood pressure and reduction of mortality, Futhan-treated rats had significantly lower levels of circulating peroxide production (P<0.05), as measured by the plasma peroxide assay compared to control animals after SAO shock. There were no significant differences between groups before the shock treatment Despite the decrease in the Futhan-treated group compared to controls after SAO shock, Futhan pretreatment was unable to prevent an increase in peroxide production after SAO shock. Circulating peroxide production was significantly higher in Futhan-treated and the saline-treated control groups after SAO shock compared to circulating values before the shock protocol (P<0.005).

Mortality in the saline-treated group was almost immediate, with the exception of one animal which survived for some time longer. None of the Futhan-treated group died during the 60 minute post-injection observation period and MAP returned to nearly initial values. Pretreatment with Futhan by infusion pump at concentration of 3.3 mg/kg body wt/hr results in a depression of MAP, as discussed above. Injection of low molecular-weight pancreatic homogenate was not lethal to any of the animals (n=4) studied.

8.4 Discussion

Intravenous infusion of Futhan mitigates the production of circulating peroxides in SAO shock. Futhan pretreatment increases systemic blood pressure and survival time in response to SAO shock.

### EXAMPLE 9

### Isolation Studies on Pancreatic Homogenate

Summary

The discovery of neutrophil activating factors from the pancreas has prompted the search for the identity of these agents. Because of the pancreas' unique position as source for catabolic digestive proteases and zymogen precursors, it is appears that pro-enzyme peptide remnants or other small degradation productions from the pancreas may function as low-molecular weight (<3 kD) neutrophil activators. The pancreas is the source of other low-molecular weight species that may also be involved in neutrophil upregulation in vivo, in particular platelet activating factor (PAF) and PAF-like substances, which have been shown to be produced in the pancreas. Low-molecular weight rat pancreatic homogenate was separated by FPLC and high performance liquid chromatography (HPLC) fractionation and analyzed by mass spectroscopy.

A definitive identification was not made but activating factors appear to be between 300-800 D molecular weight. The activator is probably composed of several different factors.

9.1 Introduction

From in vivo and in vitro experiments, it was shown that the action of the neutrophil activating factors can be inhibited by the application of serine protease inhibitors, most notably Futhan. These results suggest the participation of pancreatic digestive proteases as neutrophil activators. Inhibition by Futhan of pancreatic homogenate in vitro may be due in part to inhibition of neutrophil activation of the neutrophil itself. In vivo protection by Futhan against neutrophil activation may be achieved by stabilization of the pancreatic lysosomes and acinar cells in addition to direct neutrophil downregulation. Recovery of neutrophil activating activity in the low-molecular weight fractions of shock plasma and pancreatic homogenate indicates that there are other factors involved. A systematic approach was made to identify and/or eliminate possible (especially low-molecular weight) factors from the pancreas that may function as neutrophil activating substances.

9.1.a. Peptides

Because of the prodigious quantity of proteases located in the pancreas, often in a zymogen form, it appeared reasonable to suspect these factors as neutrophil activating substances. In particular, neutrophil activation by the low-molecular weight (<3 kD) pancreatic homogenate fraction suggested that an activator could be one of the many pro-peptides cleaved upon activation of the main protein. In addition to the large variety of pro-enzymes in the pancreas that can lead to a multitude of peptide products, there are also many different proteolytic enzymes that may be involved in pro-enzyme peptide cleavage. Each of these proteolytic enzymes cleaves preferentially at different sites in amino acid chains, giving rise to a vast number of possible peptide sequences. This can result in a surfeit of peptide permutations that would be extremely difficult and costly to analyze individually. To aid in analysis of the identification of possible peptide activators, a computer program was written to analyze different possible peptide permutation products and compare them with suspected molecular weights as determined by mass spectroscopy.

9.1.b Platelet Activating Factor (PAF) and PAF-Like Substances

Platelet activating factor (PAF) is a small amphipathic lipid that is known to mediate a wide variety of biological effects at concentrations as low as 10⁻¹⁰ M (1). In vitro PAF aggregates platelets, is also chemotactic to neutrophils and is a moderate inducer of the respiratory burst (see Example 6). PAF infusion has results in hypotension and shock in laboratory animals and acute pancreatitis when injected into the superior pancreaticoduodenal artery of rabbits. PAF has been implicated in the pathology of different disease conditions such as sepsis and shock. In particular, PAF has been postulated to be a primary factor in the course of splanchnic arterial occlusion (SAO) shock. PAF has been measured in pancreatitis where it is thought to be involved in neutrophil activation, although one study was unable to find evidence of PAF in acute conditions. The pancreas has also been shown to produce PAF in vitro, as have many other tissues in response to stimulators.

Results from the application of PAF antagonists in inflammatory conditions have been mixed, and the role PAF plays in inflammation clinically is still uncertain. Some investigators have found plasma levels of PAF or lyso-PAF (an indirect indicator of PAF concentration) to actually be decreased clinically in patients suffering from sepsis and other diseases. Whether PAF is actually circulating or remains bound during different pathologic (ischemic) events is also unclear, as well as whether PAF functions as a primary mediator or is secondary in response to other activator.

Platelet activating factor (1-O-alkyl-2-acetyl-sn-glycero-3-phosphocholine) is a class of bioactive phospholipids composed of a glycerol backbone with an O-alkyl ether group at the sn-1 position, an acetate group at the sn-2 position, and a phosphocholine at the sn-3 position. Approximately 95% of PAF compounds have 16 or 18-carbon saturated chain at the sn-1 ether linkage. Unsaturated ether groups have been detected but exhibit lower potency. The acetate group is also important for PAF bioactivity and increasing the chain length to more than 3 carbons diminishes bioactivity Hydrolysis at the sn-2 position to a hydroxyl group (HO—) results in the formation of lyso-PAF and subsequent loss of bioactivity. Lyso-PAF is the principal degradation product of PAF as well as its precursor under inflammatory conditions (McIntyre et al. (1995) Chapter 13 in Physiology and Pathophysiology of Leukocyte Activation, Graner et al., Eds., Oxford Press, Oxford, pp 1-30; and Anderson et al. (1991) Surg Gynecol Obstet 172:415-424). PAF can be formed de novo or by a remodeling pathway (see, e.g., Prescott et al. (1990) Thromb Haemost 64:99-103). The de novo synthesis pathway is the mechanism for PAF formation under quiescent conditions.

In response to inflammation, the remodeling pathway is stimulated. It is thought to be the primary route for PAF production due to inflammatory mediators (Anderson et al. (1991) Surg Gynecol Obstet 172:415-424). In the remodeling pathway phospholipase A₂ first hydrolyzes the sn-2 fatty acyl group from alkyl choline phosphoglycerides (Prescott et al. (1990) Thromb Haemost 64:99-103) to form lyso-PAF, which can then be transformed to PAF by the action of an acetyltransferase. PAF is degraded in the reverse manner by PAF acetylhydrolase, a phospholipase A₂ that only cleaves short-chain groups (Snyder et al. (1985) Adv Prostaglandin Thromboxane Leukot Res 15:693-696; and Stafforini et al. (1997) J. Biol Chem 272:17895-17898). In vitro, PAF can be degraded at the sn-2 position by phospholipase A₂, and at the sn-3 position by phospholipase C (McIntyre et al. (1995) Physiology and Pathophysiology of Leukocyte Activation Oxfor Press, Oxford 1-30). Adequate concentrations of PAF acetylhydrolase in vivo are presumably responsible for PAF's short half-life in plasma of less than 30 minutes. Plasma-derived PAF acetylhydrolase can be oxidatively inactivated, a scenario that might be of physiological importance in reperfusion injury. Organ homogenates activated with serine proteases and pancreatic homogenate, tissue PAF acetylhydrolase activity is also sensitive to trypsin cleavage. Plasma-borne PAF acetylhydrolase, however, is resistant to trypsin treatment. Mechanisms for the production of PAF-like substances in serine protease-activated homogenates (including pancreatic homogenate) may involve the degradation of PAF acetylhydrolase, resulting in increased concentrations of PAF and PAF-like substances. Whether plasma PAF acetylhydrolase is sufficient to block the potential formation of PAF-like substances from inappropriate concentrations of circulating proteases is unknown.

PAF-like substances are small lipids whose vasoactivity mimics that of PAF. Although these substances tend to be less active than PAF, often by several orders of magnitude, they function in the same manner by binding to PAF receptors and are co-localized on thin layer chromatography (TLC). Because of these similarities, reports purporting to measure PAF inhibition by inhibitors or PAF concentration by bioassays can unwittingly measure PAF-like substances instead. This is an important distinction because PAF-like substances are most likely derived from oxidative mechanisms rather than through enzymatic pathways. The critical difference, especially in the diseased state, is that the production of PAF is tightly controlled, whereas PAF-like substances are the products of unregulated inflammation. Authentic PAF, even when produced by inflammatory mediators such as large concentrations of hydrogen peroxide (1 mM), remains bound to the endothelium. By contrast, PAF-like substances are expressed when endothelium is subjected to lower concentrations of H₂O₂ for longer periods of time (at least one hour) or lipid-soluble peroxides such as tert-butylhydroperoxide (t-BuOOH). Endothelial cells treated with t-BuOOH produce large membrane blebs in response to oxidative stress. These blebs appear to be much like those seen in vitro when neutrophils are incubated with pancreatic homogenate. The production of endothelial blebbing can be blocked in vitro by the application of free radical scavengers, providing further evidence of an oxidative mechanism for their formation (McIntyre et al. (1995) Physiology and Pathophysiology of Leukocyte Activation Oxfor Press, Oxford 1-30). Like PAF, PAF-like lipids are subject to degradation at the sn-2 and sn-3 positions by phospholipase A₂ and phospholipase C, respectively. In addition, these substances can also be degraded by phospholipase A₁ at the sn-1 position, indicating the presence of an ester bond in this position rather than the ether bond of authentic PAF. PAF-like substances are believed to be formed by oxygen free radical-mediated cleavage of cell membrane constituents (phosphatidylcholine) at numerous points on the unsaturated (arachidonate) sn-2 position. (See Example 3 section 3.1.c Lipid Peroxidation for an in-depth discussion of mechanisms of oxygen free radical-mediated lipid peroxidation reactions).

9.1.c Endotoxins

Endotoxin leakage is believed to be the cause of cardiac failure in hemorrhagic and intestinal shock in dogs. There is considerable speculation about the effects of endogenous gut endotoxins and the gram-negative bacterial peptide fMLP on the course of circulatory shock. Although it is generally agreed that endotoxin translocation does play a role in the pathogenesis of these conditions, the extent of its contribution is unclear.

In the studies herein, larger endotoxins may have been present in whole molecular weight pancreatic and protease-treated homogenates. The application of antibacterial agents (see Example 5) and the finding of low-molecular weight activators much smaller than the endotoxins strongly suggests endogenous endotoxins are not among the pancreatic and protease-treated homogenate neutrophil activating factors found in these studies. The bacterial peptide fMLP was not found in any mass spectroscopy analysis.

9.2 Methods

9.2.a Methods: FPLC Separation

To identify the neutrophil activating factors present in the pancreas, rat pancreas were collected, homogenized, and incubated as described in Example 5. Pancreatic homogenate was filtered by centrifugation at 500 G and the filtrate was collected and ultrafiltered through a 3 kD cut-off filter as described in Example 5. 100 μl of pancreatic ultrafiltrates were separated using ion exchange fast pressure liquid chromatography FPLC® (gradient programmer GP-250, liquid chromatography controller LCC-500, Pharmacia LKB Biotechnology, Uppsala, Sweden). Samples were injected through either MonoQ® HR5/5 or MonoS® HR5/5 columns at a 1 ml/min flow rate. Buffer A for the MonoQ column was 20 mM Tris-HCl (pH: 8.0) and Buffer B was equal to Buffer A+ 1 M NaCl. Fractions were eluted using a standard solute elution of 0-35% Buffer B in 15 ml, 25-100% Buffer B in 10 ml, and 100% Buffer B for 5 ml. The MonoS elution was performed using the same profile, with the substitution of 50 mM acetate-NaOH (pH: 5.0) in place of the Tris-HCl as Buffer A. Aliquots were taken of representative peaks and assayed for neutrophil activation using the pseudopod formation test as described in Example 2. Although activation was demonstrated in samples from cationic as well as anionic columns, the anionic preparation displayed a more defined elution profile and was used for subsequent purification by HPLC.

9.2.b Methods: RP-HPLC Separation

Reversed-phase high performance liquid chromatography (RP-HPLC) was performed on a purified pancreatic homogenate fractions separated by FPLC MonoQ column (fractions #2-3) displaying substantial neutrophil activation activity. 250 μl samples were injected (Waters M-45, with automated gradient controller, Lambda-Max Model 480 LC spectrophotometer, Millipore Co., Milford, Mass.) using 0.1% trifluoroacetic acid (TFA) as Buffer A and 0.1% TFA+ 80% acetonitrile as Buffer B. Fractions were eluted using a solute elution of 0-10% Buffer B in 4.5 ml, 10-40% Buffer B in 1.5 ml, 40-45% Buffer B in 2.25 ml, 45-100% Buffer B in 5.25 ml, and 100% Buffer B for 1.5 ml at a 0.5 ml/min flow rate. Peaks were detected by increase in absorbance at 215 nm and elution fractions were collected manually. Before application of fractions to naive neutrophils for pseudopod formation tests purified homogenates containing HPLC solvents were volatilized under nitrogen gas.

9.2.c Methods: Matrix-Assisted Laser Desorption Ionization (MALDI) Mass Spectroscopy

FPLC and HPLC fractions containing neutrophil activation activity were processed by matrix-assisted laser desorption ionization (MALDI) mass spectroscopy. MALDI is a standard well known method for the analysis of proteins and peptides without the need for extensive purification. The matrix used was sinapinic acid (trans-3,5-dimethoxy-4-hydroxycinnamic acid, MW 224 D), which is a preferred matrix for samples containing water-acetonitrile mixtures, as the HPLC fractions contained (see, e.g., Beavis (1996) Methods in Enzymol 270:519-551). Ultra-filtered (<3 kD) rat plasma collected before and after SAO shock was also measured by MALDI. Differences in rat shock plasma spectra were plotted using MATLAB software package (The Math Works, Inc., Natick, Mass.).

9.2.d Methods: Neutrophil PAF Inhibition Experiments

To determine whether the pancreatic neutrophil activating factors and those tissue homogenates incubated with proteases were PAF-related, actin polymerization and superoxide formation tests were made using Phospholipase C (phosphatidylcholine cholinephosphohydrolase Type XI: from B. cereus suspended in 3.2 M (NH₄)₂SO₄ pH: 6.0, Sigma Chemicals, St. Louis, Mo.), an enzyme with non-specific PAF inhibitor characteristics, as well as commercial PAF-inhibitors. The PAF inhibitors used were 10 μM (±)-trans-2,5-Bis(3,4,5-trimethoxyphenyl)-1,3-dioxolane (Dioxolane) (Cal BioChem, San Diego, Calif.) and WEB2170 (Boehringer Ingelheim, Germany) in concentrations of 5 μM, 50 μM, and 500 μM. Phospholipase C concentrations used were 0.2U/ml, 1 U/ml, and 2 U/ml and did not interfere with neutrophil activation (Wazny et al. (1990) Eur J Clin Microbiol Dis 9:830-832; Lin et al. (1997) Respiration 64:96-101; and Styrt et al. (1989) J Lab Clin Med 114:51-55). Phospholipase A₂ (from bovine pancreas, Sigma Chemicals, St. Louis, Mo.), which also degrades PAF, was assayed (concentrations: 1 U/ml, 10 U/ml, 20 U/ml) but was nacceptable due to its neutrophil stimulatory properties (Hazlett et al. (1990) Adv Exp Med Biol 279:49-64; Langholz et al. (1990) Prostaglandins Leukot Essent Fatty Acids 39:227-229; Cicala et al. (1993) Gen Pharmacol 24:1197-1202).

Rat pancreatic homogenates were prepared as described in Example 5 and other organ homogenates of liver, spleen, intestine, and heart were prepared by incubation with trypsin (1300 U/ml homogenate) or chymotrypsin (52 U/ml homogenate) as described in Example 7. PAF inhibitors were incubated with tissue homogenates for 30 min at 37°. Phospholipase C was incubated with tissue homogenate for 10 min at room temperature.

Lucigenin-enhanced superoxide production from human donor plasma (neutrophil concentration 150×10³ cells/ml) was measured as described in detail in Example 6 using 1 ml of filtered homogenate, either in the presence or absence of phospholipase C.

9.2.e Methods: Peptide Sorter Computer Program

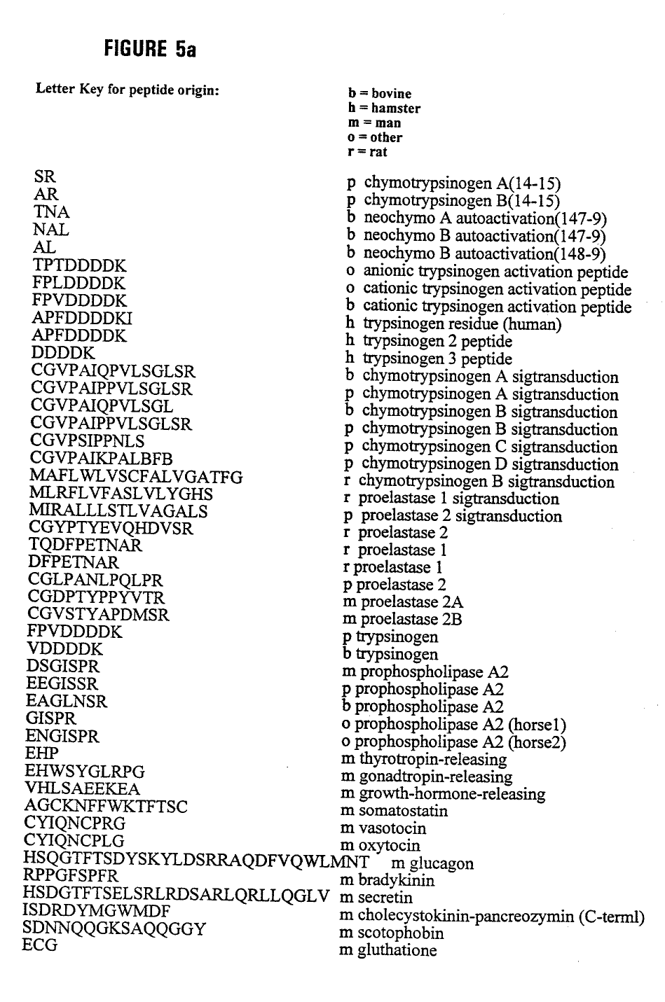

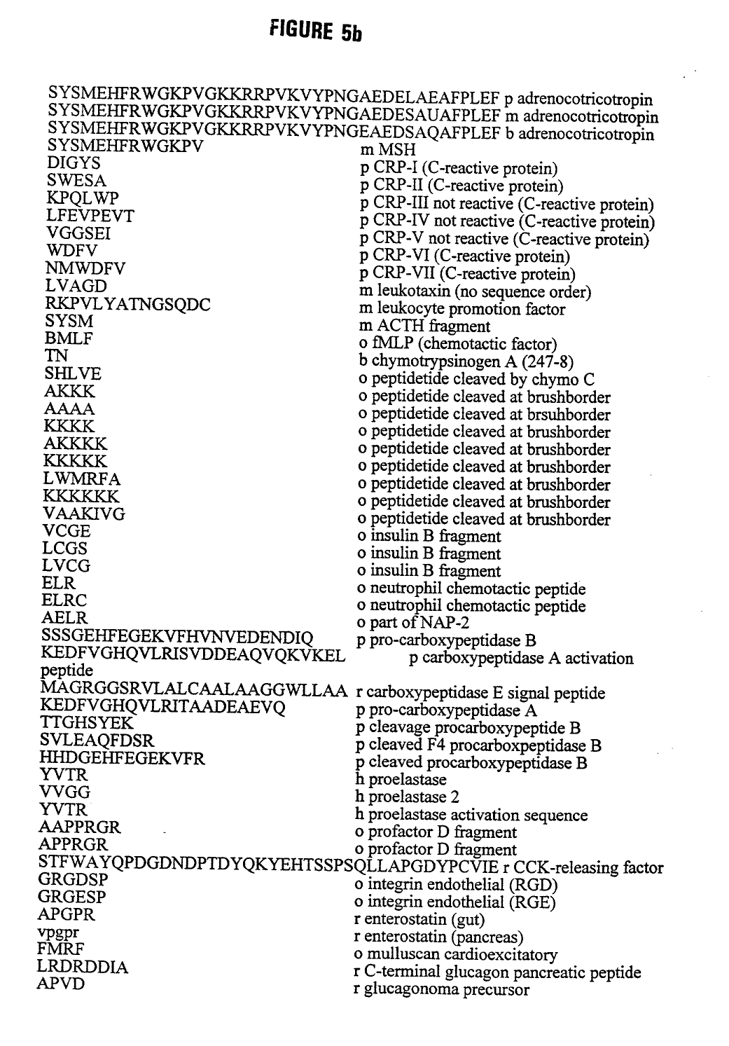

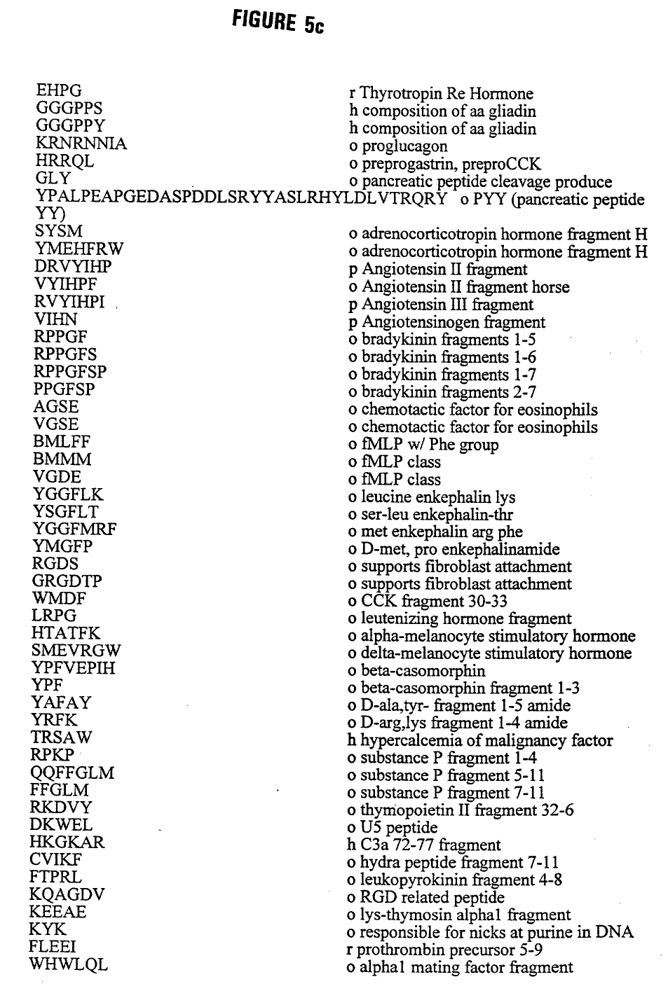

In order to identify the factors isolated from the pancreas, a computer program was written to analyze different possible peptide permutation products and compare them with suspected molecular weights as determined by mass spectroscopy. The program, which was written in FORTAN77, reads in amino acid sequences and compares them with unknowns that can be read in and compared for homology. The program was modular and menu-driven for easy modification and access. The user can either input a suspected peptide sequence, known mass, request similar peptides from a given species, or look for identical peptides and the program will calculate possible peptide masses, peptides in the neighborhood of the inputted mass, or species as requested. In the event a suspected peptide sequence is inputted, after analysis the program gives the user the option to add that peptide to the data base. In this way, each successive addition to the list of potential peptide contributors is compared with those already on the list to avoid duplication. When searching by molecular weight, a centroid about the molecular weight is computed which can be modified by the user to find other peptides with a molecular weight within a specified radius. Finding peptides that match suspected neutrophil activating factor molecular weights is a lengthy and literature-dependent process. The converse, i.e., identifying every peptide sequence that corresponds with suspected activating factor molecular weights, results in the identification of several hundred peptide sequences, which are impossible to test individually. A table of peptides tested is listed in FIG. 5. The majority of peptides tested are of pancreatic origin but other ubiquitous peptides (e.g., bradykinin, fMLP) are also included. These peptides were analyzed sequentially along the length of the peptide for similarities to suspected neutrophil activating factor molecular weights, not only of the complete peptide sequence, but also of its amino acid components.

9.3 Results

9.3.a FPLC Elution Results

FPLC of whole and low-molecular weight rat pancreatic homogenate through cationic MonoS® columns resulted in a majority of the sample eluting in the first four fractions. Most of neutrophil stimulatory activity was seen in these four fractions as well, as assayed by actin polymerization and superoxide formation tests. In particular, fraction number 3 displayed considerably greater activation by both tests than other fractions in the whole pancreatic homogenate sample while fractions #1-3 in the low-molecular weight sample resulted, in descending order, in the greatest degree of activation.

Elution of pancreatic homogenate through FPLC anionic MonoQ® columns displayed a much greater degree of separation than seen in the cationic columns, suggesting the pancreatic factors are either uncharged or slightly cationic in nature. Pancreatic homogenate injected into FPLC anionic MonoQ® columns also displayed a wider degree of scatter of neutrophil activating properties than that seen in the cationic column fractionation, in the whole and low-molecular weight fractions.

In the whole pancreatic homogenate fractions (n=3 pancreas) the greatest degree of actin polymerization resulted from neutrophils that were treated with elution from fractions #1-6, which generally corresponded with those fractions that displayed the highest level of superoxide production as well. Fractions #8-10, while not demonstrably excitatory for neutrophils via the actin polymerization test, displayed an atypical early increase in superoxide production followed by a subsequent decay well below control values. The cause of this response is unknown but suggests the presence of an additional activating factor with a different activation response than that typically measured (see Example 6). The low-molecular weight anionic column fraction (n=3 pancreas) displayed the greatest degree of neutrophil actin polymerization in the first six fractions, consistent with results from the whole molecular weight homogenate. Superoxide production was greatest in fractions #2-7, which corresponds with activation seen by pseudopod formation tests for low-molecular weight pancreatic homogenate. The early-phase superoxide production seen in fractions #8-10 of the whole homogenate elution was not detected in the low-molecular weight fraction, suggesting that the source of superoxide production in those fractions is a larger molecular weight product. Small increases compared to control neutrophil pseudopod formation seen in the higher number fractions (10-14) from cationic and anionic column FPLC elutions are almost certainly attributable to the increase in solvent salt concentration, which can give rise to indeterminate activation. This supposition is supported by superoxide measurements, which were uniformly low in these elution fractions in all pancreatic samples tested.

9.3.b Reversed Phase-HPLC Elution Results

Fraction number 2 from one of the low-molecular weight anionic FPLC column separations was further separated by RP-HPLC fractionation, and these samples were measured for their ability to activate naive neutrophils as assayed by the actin polymerization pseudopod formation test. The vast majority of activity occurred in the later fractions (#16-1 8). Although the solvent that elutes at this time is equal to 100% of Buffer B (0.1% TFA+80% acetonitrile) which is a potent detergent stimulus for neutrophils, prior volatilizing with nitrogen gas would have removed most of the noxious elements of the media. This occurred as evidenced by the lack of neutrophil activation observed in sample #15, which displays a large HPLC elution peak at 100% of Buffer B yet does not appreciably activate naive cells.

9.3.c Mass Spectroscopy Results

MALDI mass spectroscopy results from low-molecular weight rat shock plasma, FPLC separated rat pancreatic homogenate and HPLC separated rat homogenate were, for the most part, indeterminate. Mass spectroscopy of low-molecular weight rat shock plasma, obtained before and after shock display a similar spectrum. Several differences, however, were seen from a direct comparison of the two spectrums from an isolated segment of the two plots. In the absence of a focus at a particular molecular weight, the myriad differences between the spectra of plasma collected before and after shock are difficult to interpret.

The mass spectra of low-molecular weight pancreatic homogenate was analyzed, as obtained from FPLC MonoQ elution peaks #2 and #3. These two peaks show strong homology with each other, in particular a sequence of molecular weight peaks between 611 and 696 D, with an additional two coincident peaks at 471 and 879. Because there a multitude of potential peaks, it was difficult to interpret whether these peaks are related to mass spectroscopy molecular weights of the shock plasma measurements. MALDI mass spectroscopy was then performed on RP-HPLC fractions obtained from FPLC fractions #2-3 of filtered rat pancreatic homogenate that displayed neutrophil activation activity (peaks #16 and #17). These spectra display similar (to each other) molecular weight peaks, which correspond in part with peaks seen in rat plasma spectra, but not necessarily with those obtained from FPLC mass spectroscopy measurements. Peak #17, which elutes at 100% Buffer B, displays an ordered set of molecular weight peaks between approximately 991-1607 D which is not found in peak #16 which elutes just prior. This is believed be a series of detergent peaks associated with HPLC and is not interpreted as signal.

A MALDI mass spectra of a second HPLC assay fraction containing high levels of neutrophil activation activity but eluted earlier on the HPLC column (fraction #7 of 14 fractions) displays the exact spectra. It is unclear why this pattern of molecular weight peaks is not found in the mass spectrum of HPLC fraction #16, which would be expected if as a detergent, this chemical were ubiquitously present. Because these peaks are not present in FPLC mass spectra, they may be HPLC artifact.

9.3.d PAF Inhibition Results

Incubation of initially non-stimulatory organ homogenates of heart, liver, spleen, and intestine with the serine proteases trypsin or chymotrypsin resulted in marked increases in pseudopod formation compared to controls, as described in Example 7. Pancreatic homogenate also substantially activated neutrophils as has been previously shown. Application of the non-specific PAF-inactivator phospholipase C resulted in a decrease in percent pseudopod formation due to trypsin-activated homogenates, with marked decreases seen in activated spleen and liver. Pancreatic homogenate-induced neutrophil activation decreased by more than a third, and there was minimal effect upon trypsin-activated heart homogenate. Attempts at inhibiting the effect of activated homogenates on neutrophil pseudopod formation by the application of the commercial PAF inhibitor Dioxolane proved less effective.

To determine whether phospholipase C inhibition of neutrophil actin polymerization by activated homogenate would also reduce superoxide production in response to these stimuli, lucigenin-enhanced plasma chemiluminescence was tested. These tests confirm the results obtained by the pseudopod formation assays as steady-state chemiluminescence of trypsin-incubated homogenates was decreased by the addition of phospholipase C. Trypsin-incubated spleen and heart homogenate plasma chemiluminescence was decreased in comparison with control values, in contrast to neutrophil pseudopod formation assays. The reasons for this decrease are unclear.

A third PAF inhibition experiment was carried out, measuring the change in neutrophil pseudopod formation in response to trypsin-activated (n=2) and pancreatic (n=2) homogenate with and without the PAF inhibitor WEB2170. Results from WEB 2170 application at concentrations of 5 μM (n=2), 50 μM (n=2), and 500 μM (n=2) proved largely ineffective at reducing neutrophil activation in response to pancreatic or trypsin-incubated homogenates.

9.4 Discussion

The search for neutrophil activating factors that are present in plasma after inflammatory conditions such as shock and those derived from the pancreas ultimately led to attempts to isolate and conclusively identify the factors. The plasma concentrations of such activators appear to be extremely low and contaminated with other factors, including later-phase known activators such as cytokines. Therefore, concentration was directed on isolating the neutrophil pancreatic factors, which are presumably present in much higher concentrations and are limited only by the amount of tissue homogenate available. Isolation of these factors in the pancreas has not been straightforward. It appears from mass filterization results that there are probably high(er) molecular weight neutrophil activating factors in pancreatic homogenate as well as in the low molecular weight below 3 kD. Even with the focus on low molecular weight activators, there appear to be numerous factors that may activate neutrophils in vitro coming from the pancreas. Because of the particular proteolytic characteristics of the pancreas and the nature of small molecular weight species in general, focus was directed to two classes of potential activators that may be present in pancreatic homogenate.

The first of these are peptide fragments, which by virtue of their low-molecular weight and availability are a possible class of activators. Short peptides have been shown to activate neutrophils; the most notable class are the bacterial-derived fMLP groups (Schiffmann et al. (1975) Proc Natl Acad Sci USA 72:1059-1062). Recently, a three-peptide fragment released from plasma in response to alkali has been isolated activates neutrophils in vitro (Pfister et al. (1993) Invest Ophthalmol Vis Sci 34:2297-2304; and Pfister et al. (1996) Invest Ophthalmol Vis Sci 37:230-237). Pancreatic peptides are known to circulate in shock and other pathologies (Merriam et al. (1996) J Surg Res 60:417-421; Katz et al. (1964) Archives of Surgery 89:322-331; Foitzik et al. (1995) Dig Dis Sci 40:2184-2188; Leffler et al. (1973) Am J Physiol 224:824-31; Glenn et al. (1971) Circ Res 29:338-49; Lefer et al. (1970) Circ Res 26:59-69; Herva et al. (1970) Scand J Gastroenterol Suppl 8:44-52; and Lefer et al. (1970) Am J Physiol 218:1423-1427) and could thus be responsible for systemic neutrophil activation in shock.

Therefore, in an attempt to characterize components of the partially purified pancreatic homogenate, a literature search was made of predominantly pancreatic peptides, especially pro-enzyme fragments, that may be cleaved and released in the pancreas in trauma or in response to other stress situations. A computer program was written to analyze the number and sequence of these amino acids to determine which correspond to known neutrophil activators, as determined by molecular weight analysis. To take full advantage of this capability, the absolute molecular mass of the unknown activator must first be determined. To determine the molecular weight of unknown neutrophil activators MALDI mass spectroscopy was performed on rat shock plasma before and after shock. This proved to be a naive approach, as the number of major differences between pre- and post-shock spectra was minimal, while the number of smaller differences in the mass spectra was extremely large. Therefore, unless the activator is either known a prior is present in sufficient concentration, it was difficult to make a definitive determination by this method.

Pursuing an alternative approach, rat pancreatic homogenate was obtained, filtered through a 3 kD cut-off filter and separated via FPLC, and then HPLC. These elutions resulted in fewer peaks from which to make a molecular weight determination but became more difficult to quantify as the amount of sample processing increased. Because a bioassay is used in the determination of neutrophil activating factors, it is imperative that the stimulant be as physiological in nature as possible. In addition to computer analysis of possible neutrophil-activating peptide sequences, the degradation products from the two principal pancreatic serine proteases, trypsin and chymotrypsin, were evaluated explicitly. The results from these experiments are discussed in Example 7. Neither cleavage of trypsinogen by trypsin or chymotrypsin nor the cleavage of chymotrypsinogen by trypsin or chymotrypsin resulted in the formation of neutrophil-activating peptides as assayed by the actin polymerization test.

The results indicate that the pancreatic homogenate low-molecular weight component responsible for neutrophil activation is composed of a number of factors. Support for this is derived in part by the inability of any single inhibitor to control completely the inflammatory profile seen with neutrophil upregulation. All pancreatic fractions containing activity that separated through the FPLC cationic MonoS column eluted in the first four fractions, suggesting that the unknown pancreatic activators are either uncharged or slightly cationic themselves. Elution through the anionic MonoQ column, which resulted in a separation of elutants, also resulted in a separation and subsequent diminution of neutrophil activation per sample. This suggests that the neutrophil activation response is additive in nature toward these activators (e.g., the presence of priming factors). Further purification by RP-HPLC also resulted in incomplete isolation. The neutrophil activating factors eluted by HPLC were uniformly in the later fractions (#16-17). These factors, however, represent only a fraction of the original neutrophil activation response, fractionated as they are from FPLC.

The second class of potential neutrophil activators that might be produced in pancreatic homogenate are the PAF-like substances. It has already been ascertained that there does not appear a peak in any mass spectra studied to date corresponding to authentic PAF. There is however, the possibility that PAF-like substances may be functioning as neutrophil activating factors produced by the pancreas. It is possible that the mode of efficacy for PAF inhibitors is not the inhibition of PAF per se, but neutrophil activation in response to PAF-like substances that also bind to neutrophil PAF receptors. These factors have been reported to circulate in vivo, and PAF-like substances, probably originating from cell membrane lipids such as phosphatidylcholine, have been found in the plasma of smokers and animals subjected to cigarette smoke. Their production is inhibited by antioxidants (Lehr et al. (1997) J Clin Invest 99:2358-64), suggesting oxidative mechanisms in their formation.

Among the putative PAF-like substances, either produced via oxidative disruption of phosphatidylcholine or otherwise, are linoleate and arachidonate, which have been reported to stimulate superoxide formation of neutrophils at approximately 10 μM concentration (Sato et al. (1986) Physiol Chem Phys Med Nmr 18:79-87). These species have been reported to lose their ability to activate neutrophils upon oxidation, in contrast to similar studies by other investigators (McIntyre et al. (1995) Chapter 13 in Physiology and Pathophysiology of Leukocyte Activation, Graner et al., Eds., Oxford Press, Oxford, pp 1-30). Other researchers have shown that lysophospholipids such as lysophosphatidylcholine, lysoPAF, and their derivatives will potentate the neutrophil respiratory burst, but are not intrinsically reactive (Smiley et al. (1991) J Biol Chem 266:11104-11110; Lindahl et al. (1988) Scand J Clin Lab Invest 48:303-311; Ginsberg et al. (1989) Inflammation 13:163-174; Englberger et al. (1987) International Journal of Immunopharmacy 9:275-282). Related phosphocholines such as 2-azelaoylphosphatidylcholine are responsible for cell damage and membrane lysis, and may also be stimulatory towards neutrophils (Itabe et al. (1988) Biochim Biophys Acta 962:8-15). It may be that in inflammatory conditions such as ischemia, release of lipid ‘priming’ factors is sufficient to make cells hyper-responsive to any additional stimuli including other phospholipids, thus effectively functioning as activating factors themselves.

PAF-like substances capable of activating neutrophils in vitro have been found in bovine brain homogenates. These products are produced by lipid peroxidation, implicating oxidative stress as a major trigger mechanism for the production of these neutrophil activating substances (Tokumura et al. (1987) Biochem Biophys Res Commun 145:415-425; Tanaka et al. (1993) Biochim Biophys Acta 1166:264-274). Mass spectroscopy of these activators is almost exactly matched by the mass spectroscopy made from fractions #2-3 of the FPLC rat pancreatic homogenate. This offers compelling evidence that the neutrophil activating factors in the pancreas may include PAF-like substances.

The close correlation between mass spectroscopy results of FPLC active fractions and the bovine PAF-like substances, led to the subsequent neutrophil activation assays of not only pancreatic homogenate, but also protease-activated homogenates of other organs in response to PAF inhibitors. It is known that PAF formation by endothelium can be induced by thrombin, a serine protease (Bussolino et al. (1995) Eur J Biochem 229:327-337; Carveth et al. (1992) Semin Thromb Hemost 18:126-34; Zimmermann et al. (1986) Ann NY Acad Sci 485:349-368). Other investigators have found that endothelium will produce PAF in response to pancreatic proteases, and PAF production can be blocked by protease inhibitors.

In the results that show a marked increase in neutrophil activation when neutrophils are mixed with tissue homogenates that have been incubated with serine proteases, it appears that the mechanism of these actions was the in vitro formation of PAF (or PAF-like substances) in response to protease stimulation of tissue. Results from the non-specific PAF inhibitor phospholipase C suggest that neutrophil activation seen by low-molecular weight pancreatic homogenate is due in part to PAF-like substances, as phospholipase C degrades PAF-like substances in addition to PAF. This inhibition was seen in actin polymerization and chemiluminescence assays. The use of the commercial PAF receptor inhibitors Dioxolane and WEB 2170, which also competitively inhibit PAF and PAF-like factors, proved relatively ineffective at reducing neutrophil activation in response to activated homogenates.

In the studies herein, neutrophil activating factors present in low-molecular weight pancreatic homogenate were identified and partially isolated. Results from FPLC, RP-HPLC and MALDI mass spectroscopy analysis indicate that the neutrophil activating factors found in pancreatic homogenate below 3 kD are probably in the range of approximately 300-800 D. In particular, there appears to be a cluster of PAF-like substances with molecular weights of 611, 637, 655, 673, and 695 D in filtered homogenate.

9.5 Peptides

146 peptides were tested in the program and are listed by sequence with a letter indicating the species origin of the peptide, followed by a brief description of the peptide or its believed mechanism of action (see FIGS. 5a-5c). The peptide sequences were obtained from the literature as well as Sigma Chemicals and Boehringer Mannheim chemical catalogs of 1997. The majority of peptides tested are of pancreatic origin but other ubiquitous peptides (e.g., bradykinin, fMLP) are also included. These peptides are analyzed sequentially along the length of the peptide for similarities to neutrophil activating factor molecular weights, not only of the complete peptide sequence, but also of its amino acid components.

### EXAMPLE 10

### Summary of Findings and Some Conclusions

Neutrophils are implicated in the pathogenesis of a number of disease processes acute and chronic and their inappropriate upregulation is proposed herein to be a predisposing risk factor for disease in otherwise healthy individuals. Plasma taken from animals and clinically after ischemic events display the ability to activate naive neutrophils, indicating that a circulating humoral factor is in part responsible for the upregulation of neutrophils and inflammation seen after these events.

The presence of such an activator in rat shock plasma, has been identified herein. It has also been shown that it is produced endogenously by the pancreas, which, alone of all organs studied, possesses an inherent ability to activate neutrophils In vitro. Further studies were done to characterize properties of this factor in vitro and in vivo, and many of the physiological properties of the pancreatic neutrophil activator(s) have been determined.

This Example presents a summarizes the findings reported in the above Examples.

10.1 Introduction

In order to control neutrophil activation In vivo, the identity of the primary activators must first be established. It appears that primary neutrophil activators in the in vivo setting may take one of two forms: they can either be stimuli sufficient to activate neutrophils outright either by concentration or potency, or they can be lesser stimuli that only activate neutrophils that have been ‘primed’. Primed neutrophils are cells that have been subjected to a sub-activation threshold stimulus and are subsequently hyper-responsive to small concentrations of activators. A large number of factors have been identified as priming agents in vitro and this phenomenon has also been observed experimentally in vivo as well as clinically. It is quite possible that neutrophil activation in vivo is, to a large degree, dependent on the priming phenomenon. In acute conditions such as shock, there is most probably a combinatorial synergy between populations of previously quiescent and primed neutrophils.

Neutrophils circulate with varying degrees of activation. At any given moment there are circulating an activated population, a primed population, and quiescent cells, as well as presumably non-activated marginated neutrophils. In healthy individuals the majority of these cells are thought to be of the quiescent population. ‘Preactivation’ of neutrophils, is defined herein as a shifting of the neutrophil population distribution to include greater numbers of primed and activated neutrophils. This shifting of the neutrophil distribution has been correlated with increased mortality in animals subjected to hemorrhagic and endotoxic shock as well as increased lipid peroxidation levels after shock (see Example 3) that correlate with initial neutrophil preactivation. The latter result implicates a direct role by neutrophils via oxygen free radical production in mediating the increased injury seen in preactivated animals. Preactivation, while not completely understood, has been shown to be diminished by endotoxin tolerance and is probably due to a combination of factors in man. Among the factors that might influence neutrophil preactivation in vivo are sub-clinical infections, stress factors, and diet.

Studies demonstrated that fasted individuals have lower concentrations of plasma-borne neutrophil activating factors than those who have recently eaten. It was found inn control studies of autologous blood, lucigenin-enhanced chemiluminescence is influenced by the consumption of meals the previous evening containing significant amounts of lipids and fatty acids (author's observations). Work by others (Plotnick et al. (1997) Jama 278:1682-1686) has show that high lipid consumption affects forearm blood flow vasoactivity in otherwise healthy patients. This systemic decrease in vasodilation, which is inhibited by antioxidants, thus, may occur via a free radical mechanism. It may also result in the activation or priming of neutrophil subpopulations in vivo.

10.2 Conclusions

The presence of a factor produced in the pancreas that leads to neutrophil activation in vivo and in vitro has been identified. Furthermore, the presence of proteases in the pancreas has been identified as a mechanism for the production of neutrophil activating factors in otherwise non-reactive tissues. These results indicate that the pancreas appears to be a source of circulating factors, proteolytic and other, that lead to neutrophil activation in shock. Other stimuli, such as limited (sub-clinical) ischemia and dietary intake can also modify the pancreatic environment, leading to increased production of pro-inflammatory mediators in individuals whose plasma contains elevated levels of neutrophil ‘preactivation’. It is clear that the pancreas is potentially a source for neutrophil-activating factors that if not regulated, can lead to severely deleterious consequences if released into the circulation at large. These factors are likely transported through lymph channels through the thoracic duct in a manner analogous to MDF.

It is shown herein that there exists a low-molecular weight activator of less than 3 kD. This factor exhitibs an inhibition profile distinct from PAF. Some inhibition phospholipase C cleavage was observed, but little inhibition was observed using the commercial PAF inhibitors, either BTP-dioxolane and WEB 2170. Likewise, major zymogen hydrolysis fragments from trypsinogen and chymotrypsinogen were not stimulatory towards neutrophils and protease inhibition of neutrophil activation appears to be a neutrophil-mediated event and not a per se inhibition of the activating factors.

As discussed in Example 7, the factors liberated by protease incubation of homogenates other than the pancreas do not appear to be identical to those manufactured in the pancreas. It is possible that neutrophil activation seen under intravital microscopy in vivo is the result of pancreatic protease interaction with the host tissue forming activators de novo as is seen in vitro by incubation of previously non-reactive homogenates with serine proteases. Most probably, neutrophil activation seen in vivo is due to a synergistic combination of the two effects. This additive synergy between an endogenous low-molecular weight neutrophil activator and the production of new neutrophil activators by serine proteases make pancreatic homogenate a very powerful stimulus for neutrophil activation, and may be responsible for the shock seen when pancreatic complications arise clinically.

In shock, the pancreas is one of the organs to suffer most from even limited ischemia, and this ischemia may trigger the release of toxic factors into the blood. Elevated levels of circulating pancreatic proteases are routinely encountered during shock, demonstrating that pancreatic factors do circulate in the blood. In less pathologic conditions, different dietary conditions may lead to limited release of neutrophil activators such as shown seen in human plasma after fatty food intake. The concentration of neutrophil activators in the pancreas appears to be sufficient to exercise a systemic effect upon the body.

Less than 30% of a homogenized rat pancreas is enough to induce mortality within minutes when injected into a donor rat, and lower concentrations are likewise probably deleterious. In vitro, less than 1% of a homogenized rat pancreas activated isolated neutrophils with much greater strength than the same volume of 1 μM PAF or fMLP. Lastly, none of the other viscera studied possess an intrinsic ability to activate neutrophils, indicating that at least some of the circulating neutrophil activating factors measured systemically during shock and in apparent health emanate from the pancreas and are identical.

Since modifications will be apparent to those of skill in this art, it is intended that this invention be limited only by the scope of the appended claims.

What is claimed is:

1. A method of preparing a cell activating composition, comprising:
homogenizing pancreatic tissue in buffer at pH about 7 to about 8;
removing particulates;
optionally incubating the resulting homogenate with a protease;
fractionating the homogenate and selecting fractions that exhibit cell activation activity.

2. The method of claim 1, wherein the homogenate is fractionated by size and components with molecular weights of 3 kD and greater are removed.

3. The method of claim 2, further comprising subjecting the resulting homogenate to Fast Pressure Liquid Chromatography (FPLC);
and selecting and combining fractions that have cell activation activity.

4. The method of claim 3, further comprising subjecting the resulting active fractions to High Pressure Liquid Chromatography (HPLC); and
selecting and combining fractions that have cell activation activity.

5. A cell activation composition produced by the method of claim 1.

6. A cell activation composition produced by the method of claim 2.

7. A cell activation composition produced by the method of claim 3.

8. A cell activation composition produced by the method of claim 4.

9. The method of claim 1, wherein cell activation activity is assessed by measuring fee radical formation, pseudopod formation, adhesion molecule expression, granular release, production of inflammatory mediators, or any combination thereof.

10. A method of improving treatment outcome or. reducing risk of treatment, comprising:
assessing treatment options for a disease or condition by measuring cell activation levels in a subject; and, if elevated, administering activation lowering therapy prior to commencing further treatment for the disease or condition.

11. The method of claim 10, wherein cell activation is assessed by assays that measure one or more of the level of free radical production, pseudopod formation, adhesion molecule expression and degranulation.

12. The method of claim 10, wherein the disease or condition treated is selected from cardiovascular disease, inflammatory disease, trauma, autoimmune diseases, arthritis, diabetes and diabetic complications, stroke, ischemia, Alzheimer's disease.

13. The method of claim 10, wherein the treatment being assessed is surgery, treatment of unstable angina or treatment for trauma.

14. The method of claim 10, wherein activation lowering therapy comprises administering a protease inhibitor, dialysis, alterations in lifestyle to reduce stress, or alterations in diet.

15. The method of claim 14, wherein the protease inhibitor is a serine protease inhibitor.

16. The method of claim 14, wherein the protease inhibitor is selected from among α₁-proteinase inhibitor (α₁-antitrypsin), α₂-macroglobin, inter-α₁-trypsin inhibitor, and α₁-antichymotrypsin.

17. The method of claim 10, wherein the disorder is selected from the group consisting of myocardial infarction, stroke, hemorrhagic shock, diabetic retinopathy, diabetes, and venous insufficiency.

18. The method of claim 14, wherein the protease inhibitor is 6-amidino-2-naphthyl p-guanidinobenzoate dimethanesulfonate or a pharmaceutically acceptable salt, acid, ester and other derivatives thereof.

19. A method of treating or preventing disorders mediated by inappropriate cellular activation, comprising administering an effective amount of a protease inhibitor, wherein the amount is effective in lowering cell activation.

20. The method of claim 19, wherein the protease is a serine protease.

21. The method of claim 20, wherein the protease inhibitor is 6-amidino-2-naphthyl p-guanidinobenzoate dimethanesulfonate, a chymotrypsin or trypsin inhibitor or pharmaceutically acceptable salts, acids, esters and other derivatives thereof.

22. The method of claim 19, wherein the protease inhibitor is α₁-proteinase inhibitor (α₁-antitrypsin), α₂-macroglobin, inter-α₁-trypsin inhibitor, and α₁-antichymotrypsin.

23. The method of claim 19, wherein the protease inhibitor is 6-amidino-2-naphthyl p-guanidinobenzoate dimethanesulfonate or a pharmaceutically acceptable salt, acid, ester and other derivatives thereof.

24. The method of claim 19, wherein the disorder is selected from the group consisting of myocardial infarction, stroke, hemorrhagic shock, diabetic retinopathy, diabetes, and venous insufficiency.

25. An article of manufacture, comprising packaging material and a pharmaceutical composition containing a protease inhibitor, contained within the packaging material, wherein the pharmaceutical composition is effective for lowering cell activation or preventing increased cell activation, and the packaging material includes a label that indicates that the pharmaceutical composition is used for lowering cell activation levels.

26. The method of claim 25, wherein the protease inhibitor is a serine protease inhibitor.

27. The method of claim 25, wherein the protease inhibitor is selected from among α₁-proteinase inhibitor (α₁-antitrypsin), α₂-macroglobin, inter-α₁-trypsin inhibitor, and α₁-antichymotrypsin.

28. The article of manufacture of claim 25, wherein the protease inhibitor is 6-amidino-2-naphthyl p-guanidinobenzoate dimethanesulfonate or a pharmaceutically acceptable salt, acid, ester and other derivatives thereof.

29. A method for identifying compounds that lower cell activation levels, comprising:
contacting cultured cells with a composition of claim 5 and a test compound,
measuring the level of cell activation, and selecting compounds that inhibit the cell activation activity of the composition.

30. The method of claim 28, wherein the cells are endothelial cells.

31. The method of claim 28, wherein the cells are contacted with the composition prior to contacting the cells with the compound.

32. A method of diagnosis and treatment, comprising:
assessing cell activation; and, if elevated
administering activation lowering therapy.

33. The method of claim 32, wherein activation lowering therapy comprises modifications in diet and/or lifestyle.

34. The method of claim 32, wherein activation lowering therapy comprises administration of a protease inhibitor.

35. The method of claim 32, wherein the protease inhibitor is a serine protease inhibitor.

36. The method of claim 32, wherein the protease inhibitor is selected from among α₁-proteinase inhibitor (α₁-antitrypsin), α₂-macroglobin, inter-α₁-trypsin inhibitor, and α₁-antichymotrypsin.

37. The article of manufacture of claim 25, wherein the protease inhibitor is 6-amidino-2-naphthyl p-guanidinobenzoate dimethanesulfonate or a pharmaceutically acceptable salt, acid, ester and other derivatives thereof.

38. The method of claim 32, wherein activation lowering therapy comprises dialysis.

39. A method for measuring cell activation in a subject, comprising:
contacting quiescent cultured cells with a plasma from the subject, and detecting activation of the cultured cells.
